(19) United States
(12) Patent Application Publication
(10) Pub. No.: US 2025/0177559 A1
(43) Pub. Date: Jun. 5, 2025

(54) BRANCHED LIPID CHARGE-ALTERING RELEASABLE TRANSPORTERS FOR NUCLEIC ACID DELIVERY

(71) Applicant: The Board of Trustees of the Leland Stanford Junior University, Stanford, CA (US)

(72) Inventors: Harrison P. Rahn, Stanford, CA (US); Jiuzhi Sun, Stanford, CA (US); Zhijian Li, Stanford, CA (US); Ronald Levy, Stanford, CA (US); Robert M. Waymouth, Stanford, CA (US); Paul A. Wender, Stanford, CA (US)

(73) Assignee: The Board of Trustees of the Leland Stanford Junior University, Stanford, CA (US)

(21) Appl. No.: 18/844,461

(22) PCT Filed: Mar. 7, 2023
(86) PCT No.: PCT/US2023/063812

§ 371 Date: Sep. 6, 2024

Related U.S. Application Data
(60) Provisional application No. 63/269,067, filed on Mar. 9, 2022.

(51) Int. Cl. A61K 47/69; A61K 47/59; A61K 48/00; C12N 15/88

(52) U.S. Cl. CPC A61K 47/6935; A61K 47/593; A61K 48/0041; C12N 15/88

(57) ABSTRACT

There are provided herein, inter alia, complexes, compositions and methods for the delivery of nucleic acid into a cell in vivo. The complexes, compositions and methods may facilitate complexation, protection, delivery and release of oligonucleotides and polyanionic cargos into target cells, tissues, and organs both in vitro and in vivo.

### CROSS-REFERENCE TO RELATED APPLICATIONS

This application claims the benefit of priority under 35 U.S.C. § 119(e) to U.S. Provisional Application No. 63/269,067 filed Mar. 9, 2022, the entire contents of which is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under contracts CHE-1566423 awarded by the National Science Foundation and 1R01CA24553301 awarded by the National Institutes of Health. The Government has certain rights in the invention.

### BACKGROUND

Nucleic acids are polyanionic, polar, and relatively large molecules on the order of about 13 kilodaltons, compared for example to small molecule therapeutics which are generally about 1 kilodalton or less. These properties of nucleic acids prevent or significantly hinder their unassisted passage through the nonpolar lipid membranes of cells and tissues. In addition, nucleic acids, especially RNAs, are susceptible to enzymatic degradation. Two main strategies have developed to address these difficulties with delivery of nucleic acid based therapeutic agents. One approach is the development of noncharged and nonbiodegradable nucleic acid surrogates. Another approach is the development of delivery vehicles that facilitate delivery of the nucleic acids across lipid membranes. Delivery vehicles have included lipids, peptides, and aptamers as well as cationic polymers. Previous work on nucleic acid delivery has highlighted the importance of lipophilic domains on delivery vehicles such as cationic polymers to facilitate cargo binding and membrane interaction leading to cellular internalization. Geihe et al, Proc Natl Acad Sci USA, 109:13171-13176 (2012). This work was extended to provide amphipathic diblock oligomers consisting of a lipidated oligocarbonate block and a cationic α-amino ester block. McKinlay et al. Proc. Natl. Acad. Sci. U.S.A 2017; 114(4):E448-E456. However, to date, there remains a need for new materials and strategies for the targeted delivery of nucleic acids into cells. The present invention addresses this need.

### SUMMARY

The disclosure provides co-oligomers comprising non-linear branched lipophilic monomers and degradable poly(alpha-aminoester) monomers which when complexed with nucleic acid unexpectedly provide cell-type and/or tissue specific delivery of the nucleic acid cargo. The invention is based, in part, on the inventors' discovery that certain non-linear branched lipophilic monomers, as described herein, confer unexpected cell and tissue specificity to the co-oligomer, allowing for cell-type or tissue-specific targeted nucleic acid delivery. Accordingly, the invention provides co-oligomers comprising non-linear branched lipophilic monomers and poly(alpha-aminoester) monomers, complexes of the co-oligomers with nucleic acids, and related compositions and methods.

In an aspect is provided a co-oligomer complexed with nucleic acid in which the nucleic acid is non-covalently bound to a co-oligomer. The co-oligomer includes non-linear branched lipophilic monomers and poly(alpha-aminoester) monomers, as described herein.

In an aspect, provided is a co-oligomer comprising a plurality of non-linear branched lipophilic monomers (LP) and a plurality of poly(alpha-aminoester) monomers (AM), wherein the co-oligomer has a formula:

R^{1A}-[L¹-[(LP¹)_{z1}-(LP²)_{z3}-(AM)_{z2}]_{z4}-L²-R^{2A}]_{z5} (I),

or

R^{1A}-[L¹-[(LP¹)_{z1}-(AM)_{z2}-(LP²)_{z3}]_{z4}-L²-R^{2A}]_{z5} (I′)
wherein:
R^{1A} is hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are optional linkers and are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each LP¹ or LP² is independently a non-linear branched lipophilic monomer having a structure of:

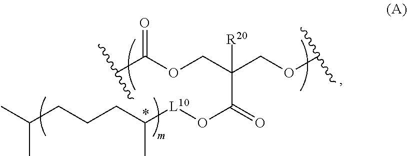

wherein:
m is an integer from 0 to 6;
L¹⁰ is independently a substituted or unsubstituted alkylene; and
R²⁰ is independently hydrogen or unsubstituted C₁-C₄ alkyl,
LP¹ and LP² are same or different;
AM is a poly(alpha-aminoester) monomer;
z5 is an integer from 1 to 10;
z1 and z3 are independently integers from 5 to 30, wherein at least one of z1, or z3 is not 0;
z4 is an integer from 1 to 100, and
z2 is an integer from 5 to 30.

In an aspect, provided is a co-oligomer comprising non-linear branched lipophilic monomers (LP) and poly(alpha-aminoester) monomers (AM), wherein the co-oligomer has a formula:

R^{1A}-[L¹-[(LP¹)_{z1}-(LP²)_{z3}-(AM)_{z2}]_{z4}-L²-R^{2A}]_{z5} (I), or

R^{1A}-[L¹-[(LP¹)_{z1}-(AM)_{z2}-(LP²)_{z3}]_{z4}-L²-R^{2A}]_{z5} (I′)
wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each LP¹ or LP² are independently a non-linear branched lipophilic monomer having a structure of:

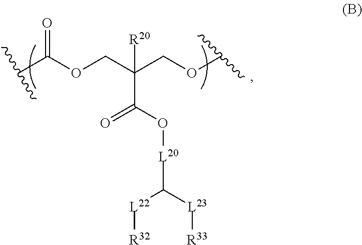

wherein:
L²⁰ is -L^{20A}-L^{20B}-;
each L^{20A} and L^{20B} is independently a bond, a substituted or unsubstituted alkylene, substituted or unsubstituted heteroalkylene, substituted or unsubstituted cycloalkylene, or substituted or unsubstituted heterocycloalkylene;
each L²², and L²³ is independently a bond, substituted or unsubstituted alkylene, or substituted or unsubstituted heteroalkylene;
R²⁰ is independently hydrogen or unsubstituted C₁-C₄ alkyl; and
each R³² and R³³ are independently hydrogen, or substituted or unsubstituted alkyl,
LP¹ and LP² are same or different;
AM is said poly(alpha-aminoester) monomer;
z1 and z3 are independently integers from 5 to 30, wherein at least one of z1, or z3 is not 0;
z4 is an integer from 1 to 100, and
z2 is an integer from 5 to 30.

In an aspect, provided is a co-oligomer of Formula I or I′ non-covalently complexed with nucleic acid. In embodiments, the nucleic acid is RNA or DNA. In embodiments, the nucleic acid is messenger RNA, small interference RNA, short hairpin RNA, micro RNA, guide RNA, CRISPR RNA, transactivating RNA, plasmid DNA, minicircle DNA, or genomic DNA.

In an aspect, provided is a nanoparticle composition comprising a plurality of co-oligomer of Formula I or I′ non-covalently complexed with nucleic acid.

In an aspect, provided is a pharmaceutical composition comprising a plurality of co-oligomer of Formula I or I′ non-covalently complexed with nucleic acid, or a plurality of nanoparticles comprising same, and a pharmaceutically acceptable carrier.
In an aspect, provided is a vaccine composition comprising a plurality of co-oligomer of Formula I or I′ non-covalently complexed with nucleic acid, or a plurality of nanoparticles comprising same, and optionally an immunological adjuvant.

In an aspect, provided is a method of transfecting a nucleic acid into a cell in vitro or ex vivo, the method including contacting a cell with a plurality of co-oligomer of Formula I or I′ non-covalently complexed with nucleic acid, or a plurality of nanoparticles comprising same.

In an aspect, provided is a method of gene editing comprising contacting a cell with with a plurality of co-oligomer of Formula I or I′ non-covalently complexed with nucleic acid, or a plurality of nanoparticles comprising same, wherein the nucleic acid comprises a first nucleotide encoding a CRISPR-Cas system guide RNA that hybridizes with a target sequence in the genome of the cell, and a second nucleotide encoding a Cas9 protein, wherein the first and second nucleotides are located in the same or different vectors.

In an aspect, provided also is a non-linear branched lipophilic monomer having a structure of.

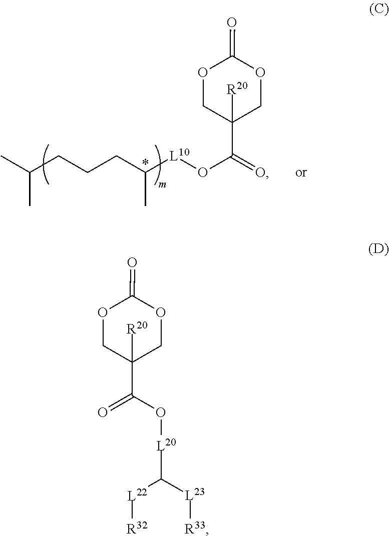

wherein
m is an integer from 0 to 6;
L¹⁰ is independently a substituted or unsubstituted alkylene;
R²⁰ is independently hydrogen or unsubstituted C₁-C₄ alkyl;
L²⁰ is -L^{20A}-L^{20B}-;
each L^{20A} and L^{20B} is independently a bond, a substituted or unsubstituted alkylene, substituted or unsubstituted heteroalkylene, substituted or unsubstituted cycloalkylene, or substituted or unsubstituted heterocycloalkylene;
Each L²², and L²³ is independently a bond, substituted or unsubstituted alkylene, or substituted or unsubstituted heteroalkylene;
each R³² and R³³ are independently hydrogen, or substituted or unsubstituted alkyl.

Other aspects are disclosed infra.

### BRIEF DESCRIPTION OF THE DRAWINGS

FIG. 1A shows an exemplary synthesis of isoprenoid based non-linear branched lipophilic monomers.

FIG. 1B shows an exemplary synthesis of isoprenoid CART from isoprenoid based non-linear branched lipophilic monomers.

FIG. 2 shows exemplary compounds of isoprenoid based non-linear branched lipophilic monomers.

FIG. 3 shows exemplary non-linear branched lipophilic monomers.

FIG. 4 shows an exemplary charge-altering releasable transporters (CARTs) with isoprenoid based lipids.

FIG. 5A shows results of fLuc mRNA transfection shown as bioluminescence normalized to ONA290 in HeLa, A549, Jurkat and B-cells using isoprenoid CARTs.

FIG. 5B shows results of fLuc mRNA transfection rate in HeLa, A549, Jurkat and B-cells using isoprenoid CARTs.

FIG. 5C shows in vivo fLuc mRNA transfection in mice using isoprenoid CARTs.

FIG. 6A shows exemplary glycerol based non-linear branched lipophilic monomers.

FIG. 6B shows exemplary charge-altering releasable transporters (CARTs) with glycerol based lipids.

FIG. 6C shows exemplary synthesis scheme for the CARTs described in FIG. 6B.

FIGS. 7A-7C show results of fLuc mRNA transfection in HeLa (FIG. 7A), Jurkat (FIG. 7B), and GM12878 B lymphoma cell (FIG. 7C) using isoprenoid CARTs.

FIG. 8A-8C show that non-linear branched lipophilic monomers undergo the rearrangement to bis-N-hydroxyethyl-2,5-piperizinedionebis-hydroxymethyl glycine. FIG. 8A shows an exemplary degradation of a model isoprenoid CART (BnO-C13A11) into a lipidated oligo-carbonate (BnO-C13) and neutral small molecule (DKP) under buffered neutral conditions. FIG. 8B shows NMR time-course of BnO-C13A11 (115 μM) degrading into DKP with a diagnostic peak at 4.14 ppm. T=0 (bottom) was taken in d6-DMSO before addition to deuterated PBS 7.4. The final spectra (top) was taken in d4-methanol following lyophilization. FIG. 8C shows quantification of DKP with respect to time. The relative integrations of the singlet at 4.14, corresponding to the DKP ring's hydrogens labelled α, and the peak for d6-DMSO at 2.67 in deuterated PBS 7.4 buffer were used to quantify the evolution DKP with respect to time. Degradation is complete after 36 minutes.

FIGS. 9A-9B show test results of CART including F18A19 compared to ONA290 for targeting spleen and for intravenous injection.

FIGS. 10A-10F show test results of CART including F11A12, F25A13, P13A11, and P22A11 compared to ONA290 for targeting spleen and for intravenous injection.

FIGS. 11A-11B show a line graph (A) of zeta potential time courses for O6N6A9, P19A19, and C13A11; and table (B) shows data for these and other transporters. See Example 7 for detail.

### DETAILED DESCRIPTION

The disclosure provides polymer-based nucleic acid delivery vehicles having specificity for certain types of cells or tissues. The compounds and compositions described here advantageously provide for cell and tissue-specific targeting of ionically complexed nucleic acid cargo delivered in vivo, as well as high efficiency nucleic acid delivery to certain specific cell types in vitro. The co-oligomers described here combine poly(alpha-aminoester) monomers with non-linear branched lipophilic monomers, which may generally be described as “isoprenoid based” or “glycerol based” monomer blocks and which confer unexpected cell and tissue specificity to the co-oligomer. In addition, the poly(alpha-aminoester) monomers undergo a unique pH-dependent intramolecular rearrangement resulting in their rapid degradation into neutral amides and small molecules at physiological pH, e.g., pH 7.3-7.4. This unique intramolecular rearrangement advantageously results in rapid intracellular release of the complexed nucleic acid cargo, as described previously in McKinlay et al., Proc. Natl. Acad. Sci. USA 2017, McKinlay et al., Proc. Natl. Acad. Sci. USA 2018, 115 (26), and WO 2018022930.

While various embodiments and aspects of the present disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be employed in practicing the disclosure.

### Definitions

Unless the context indicates otherwise, it is specifically intended that the various features of the disclosure described herein can be used in any combination. Moreover, the disclosure also contemplates that in some embodiments any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

It must be noted that as used herein and in the appended claims, the singular forms “a”, “an”, and “the” include plural referents unless the context clearly dictates otherwise. Thus, for example, reference to “a cancer cell” includes a plurality of cancer cells. In other examples, reference to “a nucleic acid” or “nucleic acid” includes a plurality of nucleic acid molecules, i.e. nucleic acids.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

As use herein the terms “oligomer” and “polymer” refer to a compound that has a plurality of repeating subunits, (e.g., polymerized monomers). The terms “co-oligomer” or “co-polymer” refers to an oligomer or polymer that includes 2 or more different residues (monomer units or monomers, which are interchangeably used herein). The number of monomers in oligomers is generally less than the number of monomers in polymers. Therefore, in embodiments, oligomers can have 1 to about 10 monomers, 1 to about 20 monomers, 1 to about 30 monomers, 1 to about 40 monomers, 1 to about 50 monomers, 1 to about 100 monomers, 1 to about 150 monomers, 1 to about 200 monomers, 1 to about 250 monomers, 1 to about 300 monomers, 1 to about 350 monomers, 1 to about 400 monomers, 1 to about 450 monomers or 1 to about 500 monomers is in length. In embodiments, oligomers can have less than about 500 monomers, less than about 450 monomers, less than about 400 monomers, less than about 350 monomers, less than about 300 monomers, less than about 250 monomers, less than about 200 monomers, less than about 150 monomers, less than about 100 monomers, less than about 50 monomers, less than about 40 monomers, less than about 30 monomers, less than about 20 monomers or less than about 10 monomers in length. In the context of polymers, the number of monomers in polymers is generally more than the number of monomers in oligomers. Therefore, in embodiments, polymers can have about 500 to about 1000 monomers, about 500 to about 2000 monomers, about 500 to about 3000 monomers, about 500 to about 4000 monomers, about 500 to about 5000 monomers, about 500 to about 6000 monomers, about 500 to about 7000 monomers, about 500 to about 8000 monomers, about 500 to about 9000 monomers, about 500 to about 10000 monomers, or more than 10000 monomers in length.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g., -A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkeny includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples o]f heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

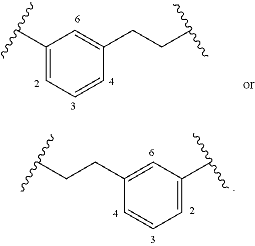 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The term “nucleophilic moiety” refers to a chemical species or functional group that is capable of donating one or more electrons (e.g., 2) to an electrophile. In embodiments, a nucleophilic moiety refers to a chemical species or functional group that can donate an electron to an electrophile in a chemical reaction to form a bond.

The term “electrophilic moiety” refers to a chemical species or functional group that is capable of receiving one or more electrons (e.g., 2). In embodiments, an electrophilic moiety refers to a chemical species or functional group that has a vacant orbital and can thus accept an electron to form a bond in a chemical reaction.

The term “oligoglycol moiety” refers to is a chemical entity with the general formula: R⁴⁰⁰—O—(CH₂—CH₂—O)n₃₀₀- [symbol] where R⁴⁰⁰ is H, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl and n300 is an integer of 1 or more. In embodiments, R⁴⁰⁰ is H or alkyl.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

“Nucleic acid” refers to deoxyribonucleotides or ribonucleotides and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA), and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids has one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g. mRNA) or altering transcript splicing (e.g. single stranded morpholino oligo). In embodiments, the nucleic acid is RNA (e.g. mRNA). In embodiments the nucleic acid is 10 to 100,000 bases in length. In embodiments the nucleic acid is 50 and 10,000 bases in length. In embodiments the nucleic acid is 50 and 5,000 bases in length. In embodiments the nucleic acid is 50 and 1,000 bases in length.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. In embodiments, contacting includes, for example, allowing a nucleic acid to interact with an endonuclease.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “stem cell” or “stem cells” refers to a clonal, self-renewing cell population that is multipotent and thus can generate several differentiated cell types.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

A “vector” is a nucleic acid that is capable of transporting another nucleic acid into a cell. A vector is capable of directing expression of a protein or proteins encoded by one or more genes carried by the vector when it is present in the appropriate environment.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/. By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, having the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “specific binding” or “specifically binds” refer to two molecules forming a complex (e.g., a ribonucleoprotein and a transfection peptide) that is relatively stable under physiologic conditions.

Methods for determining whether a ligand binds another species (e.g., a protein or nucleic acid) and/or the affinity of such ligand-species interaction are known in the art. For example, the binding of a ligand to a protein can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA).

Immunoassays which can be used to analyze immunospecific binding and cross-reactivity of the ligand include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

An exemplary immunoglobulin (antibody) structural unit can have a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “co-oligomer complexed with nucleic acid” or the like refers to a chemical complex (e.g., a complex or composition disclosed herein and embodiments thereof), which is formed with co-oligomer and nucleic acid bound to the co-oligomer and capable of penetrating into a cell (a biological cell, such as a eukaryotic cell or prokaryotic cell). In embodiments, the co-oligomer complexed with nucleic acid includes a nucleic acid ionically bound to a co-oligomer. In embodiments, the nucleic acid is unable to substantially penetrate the cell in the absence of the co-oligomer. Thus, in embodiments, the co-oligomer facilitates the transport of the nucleic acid into the cell. As used herein, the terms “cationic charge altering releasable transporter,” “CART” and the like refer to the co-oligomer disclosed herein. The CART compounds are able to release the nucleic acid component within the cell through the action of a poly(alpha-aminoester) monomer (alternatively, poly(alpha-aminoester) monomer) within the co-oligomer component, which reacts in response to an intracellular pH thereby releasing the nucleic acid with in the cell. In embodiments, the co-oligomer degrades rapidly within the cell (e.g. a half-life of less than 1 hour at pH 7.4). The co-oligomer complexed with nucleic acid may be referred to as a polyplex, a complex, an electrostatic complex, a CART/nucleic acid complex, a CART/oligonucleotide complex, a CART/polynucleotide complex. In addition, the co-oligomer complexed with nucleic acid may condense to form nanoparticles which are typically several hundred nanometers in diameter and serve to protect the nucleic acid cargo and may also further facilitate cellular entry. In addition, the co-oligomer complexed with nucleic acid may condense to form nanoparticles which are typically several hundred nanometers in diameter and serve to protect the nucleic acid cargo and may also further facilitate cellular entry. Exemplary nanoparticles formed using co-oligomers described herein are characterized in Example 7 below.

The term “amphipathic polymer” as used herein refers to a polymer containing both hydrophilic and hydrophobic portions. In embodiments, the hydrophilic to hydrophobic portions are present in a 1 to 1 mass ratio. In embodiments, the hydrophilic to hydrophobic portions are present in a 1 to 2 mass ratio. In embodiments, the hydrophilic to hydrophobic portions are present in a 1 to 5 mass ratio. In embodiments, the hydrophilic to hydrophobic portions are present in a 2 to 1 mass ratio. In embodiments, the hydrophilic to hydrophobic portions are present in a 5 to 1 mass ratio. An amphipathic polymer may be a diblock or triblock copolymer. In embodiments, the amphiphilic polymer may include two hydrophilic portions (e.g., blocks) and one hydrophobic portion (e.g., block).

The term “non-linear branched lipophilic monomer” or the like, which may also be referred to as “lipid block”, refers to a region of the co-oligomer described herein comprising a lipophilic domain of non-linear, branched hydrocarbons as described herein.

The term “initiator” refers to a compound that is involved in a reaction synthesizing a co-oligomer having the purpose of initiating the polymerization reaction. Thus, the initiator is typically incorporated at the end of a synthesized polymer. For example, a plurality of molecules of one type (or formula) of monomer or more than one type of monomers (e.g. two different types of monomers) can be reacted with an initiator to provide a co-oligomer. The initiator can be present on at least one end of the resulting polymer and not constitute a repeating (or polymerized) unit(s) present in the polymer.

The terms “disease” or “condition” refer to a state of being or health status of a subject capable of being treated with a compound, pharmaceutical composition, or method provided herein. The disease can be an autoimmune, inflammatory, cancer, infectious, metabolic, developmental, cardiovascular, liver, intestinal, endocrine, neurological, or other disease. In embodiments, the disease is cancer (e.g. breast cancer, ovarian cancer, sarcoma, osteosarcoma, lung cancer, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, neuroblastoma).

The term “infection” or “infectious disease” refers to a disease or condition that can be caused by organisms such as a bacterium, virus, fungi or any other pathogenic microbial agents.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As defined herein, the term “inhibition”, “inhibit”, “inhibiting” and the like in reference to an activity and/or functionality of a molecule (e.g. polynucleotide or protein) means negatively affecting (e.g., decreasing or reducing) the activity or function of the molecule relative to the activity or function of the protein in the absence of the inhibition. Thus, inhibition includes, at least in part, partially or totally blocking stimulation, decreasing, preventing, or delaying activation, or inactivating, desensitizing, or down-regulating signal transduction or enzymatic activity or the amount of a protein or polynucleotide. Similarly an “inhibitor” is a compound that inhibits a target bio-molecule (i.e. nucleic acid, peptide, carbohydrate, lipid or any other molecules that can be found from nature), e.g., by binding, partially or totally blocking, decreasing, preventing, delaying, inactivating, desensitizing, or down-regulating activity of the target bio-molecule. In the context of disease prevention treatment, inhibition refers to reduction of a disease or symptoms of disease.

“Treatment,” “treating,” and “treat” are defined as acting upon a disease, disorder, or condition with an agent to reduce or ameliorate harmful or any other undesired effects of the disease, disorder, or condition and/or its symptoms. “Treating” or “treatment of” a condition or subject in need thereof refers to (1) taking steps to obtain beneficial or desired results, including clinical results such as an amelioration or reduction in one or more symptoms of the disease, disorder, or condition; (2) inhibiting the disease, for example, arresting or reducing the development or clinical progression of the disease, disorder, or condition, or any one or more of its clinical symptoms; (3) relieving the disease, for example, causing regression of the disease or its clinical symptoms; or (4) delaying or slowing disease progression.

The term “prevent,” “preventing” or “prevention”, in the context of a disease, refers to causing the clinical symptoms of the disease not to develop in a subject that does not yet experience or display symptoms of the disease. In embodiments, such prevention can be applied to a subject who can be considered predisposed of the disease, whereas in some other examples, the subject may not be necessarily considered predisposed to the disease.

As used herein, “administering” refers to the physical introduction of a composition to a subject, using any of the various methods and delivery systems known to those skilled in the art. Preferred routes of administration for the composition described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, the composition described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “associated” or “associated with” in the context of a substance or substance activity or function associated with a disease means that the disease can be caused by (in whole or in part), or a symptom of the disease can be caused by (in whole or in part) the substance or substance activity or function. When the term is used in the context of a symptom, e.g. a symptom being associated with a disease or condition, it means that a symptom can be indicative of the disease or condition present in the subject who shows the symptom.

The term “subject,” “individual,” “host” or “subject in need thereof” refers to a living organism suffering from a disease or condition or having a possibility to have a disease or condition in the future. A term “patient” refers to a human that already has a disease or condition, e.g. a patient who has been diagnosed with a disease or condition or has one or more symptoms associated with a disease or condition. Non-limiting examples of subjects include humans, other mammals, bovines, rats, mice, dogs, monkeys, goat, sheep, cows, deer, and other non-mammalian animals.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infection disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

According to the methods provided herein, the subject can be administered an effective amount of one or more of agents, compositions or complexes, all of which are interchangeably used herein, (e.g. co-oligomer complexed with nucleic acid or vaccine composition) provided herein. The terms “effective amount” and “effective dosage” are used interchangeably. The term “effective amount” is defined as any amount necessary to produce a desired effect (e.g., transfection of nucleic acid into cells and exhibiting intended outcome of the transfected nucleic acid). Effective amounts and schedules for administering the agent can be determined empirically by one skilled in the art. The dosage ranges for administration are those large enough to produce the desired effects, e.g. transfection of nucleic acid, modulation in gene expression, gene-edition, induction of stem cells, induction of immune response and more. The dosage should not be so large as to cause substantial adverse side effects, such as unwanted cross-reactions, anaphylactic reactions, and the like. Generally, the dosage can vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount can show an increase or decrease of at least 5%, 1%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation can depend on the purpose of the treatment, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

Co-Oligomer Complexed with Nucleic Acid

In a first aspect, there is provided a co-oligomer complexed with nucleic acid that is non-covalently bound to the co-oligomer. In embodiments, the nucleic acid is ionically bound to the co-oligomer. In embodiments, the co-oligomer may be complexed with a plurality of different nucleic acids, including for example different plasmids or a combination of different RNA-based therapeutic agents, such as siRNA agents.

In general, the theoretical charge ratio of cationic co-oligomer to anionic nucleic acid is from about 2:1 to about 30:1 (cation:anion). In embodiments, the theoretical charge ratio is 2:1, 3:1, 5:1, 10:1, 15:1, 20:1, 25:1, or 30:1. Theoretical (+/−) charge ratios are calculated as moles of ammonium cations to moles of phosphate anions, assuming full amine protonation and phosphate deprotonation. In some embodiments, the charge ratio (+/−) is 3:1, or 5:1.

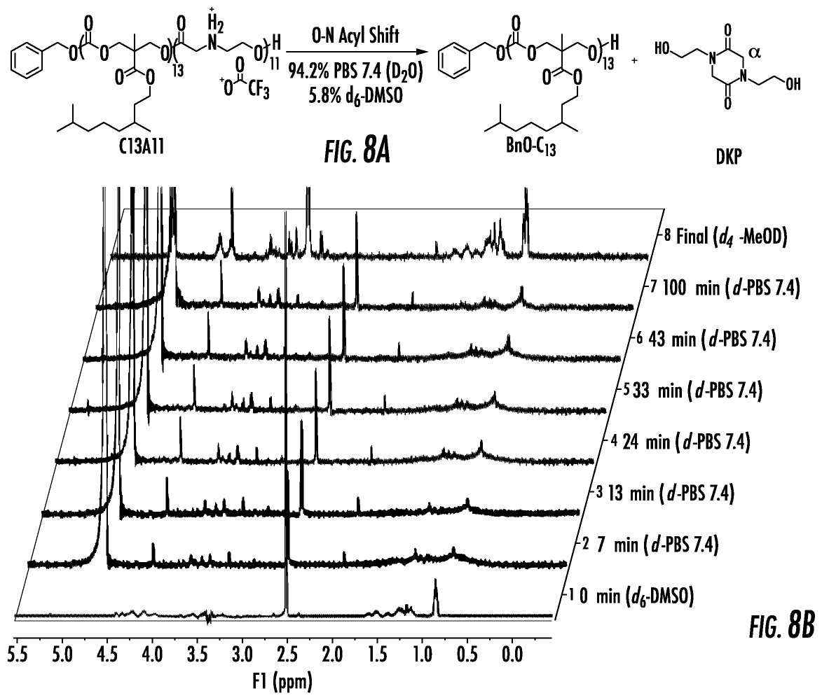

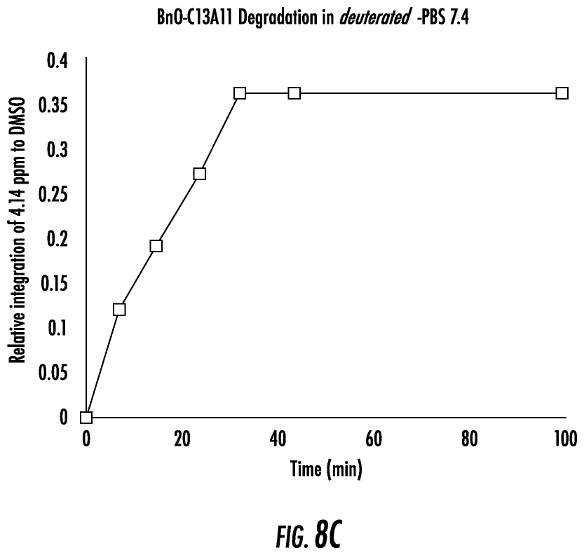

In accordance with the present disclosure, the co-oligomer comprises a cationic charge altering releasable transporter (CART) domain comprising poly(alpha-aminoester) monomers (AM). The poly(alpha-aminoester) or CART domain of the co-oligomer undergoes a unique pH-sensitive intramolecular rearrangement at physiological pH resulting in its degradation into uncharged amides and small molecules. For example, an exemplary non-linear branched lipophilic monomer, C13A11, may rearrange and degrade into bis-N-hydroxyethyl-2,5-piperizinedionebis-hydroxymethyl glycine (FIGS. 8A-8C).

In an aspect is provided a co-oligomer including non-linear branched lipophilic monomers (LP) and poly(alpha-aminoester) monomers (AM).

In embodiments, the co-oligomer has the formula (I):

R^{1A}-[L¹-[(LP¹)_{z1}-(LP²)_{z3}-(AM)_{z2}]_{z4}-L²-R^{2A}]_{z5} (I), or

R^{1A}-[L¹-[(LP¹)_{z1}-(AM)_{z2}-(LP²)_{z3}]_{z4}-L²-R^{2A}]_{z5} (I′)
wherein:
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCH₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are optional linkers and are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each LP¹ or LP² is independently a non-linear branched lipophilic monomer, and LP¹ and LP² are same or different;
AM is a poly(alpha-aminoester) monomer;
z5 is an integer from 1 to 10;
z1 and z3 are independently integers from 5 to 30, wherein at least one of z1, or z3 is not 0;
z4 is an integer from 1 to 100, and
z2 is an integer from 5 to 30.

In embodiments, L¹ is substituted or unsubstituted C₁-C₃ alkylene. In embodiments, L¹ is substituted or unsubstituted methylene. In embodiments, L¹ is substituted or unsubstituted C₁-C₆ alkylene, or substituted or unsubstituted 2 to 6 membered heteroalkylene. In embodiments, L¹ is substituted or unsubstituted C₁-C₃ alkylene, or substituted or unsubstituted 2 to 3 membered heteroalkylene.

In embodiments, L¹ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹ is unsubstituted alkylene, unsubstituted heteroalkylene, unsubstituted cycloalkylene, unsubstituted heterocycloalkylene, unsubstituted arylene, or unsubstituted heteroarylene. In embodiments, L¹ is unsubstituted alkylene (e.g., C₁-C₆ alkylene). In embodiments, L¹ is a bond.

In some embodiments, the co-oligomer complexed with nucleic acid has a co-oligomer having any of the foregoing formula in which L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is —CH₂—O—.

In some embodiments, the co-oligomer can have any of the foregoing formula in which L¹ is —CH₂—O—,

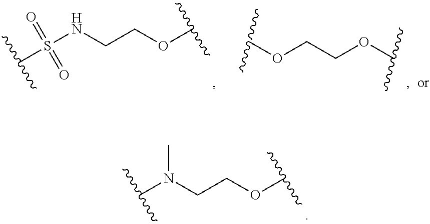

In embodiments, L is —CH₂—O—. In embodiments, L¹ is

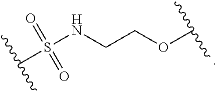 [SMILES: *OCCNS(*)(=O)=O]

In embodiments, L¹ is

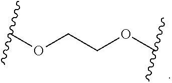 [SMILES: *OCCO*]

In embodiments, L¹ is

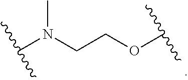 [SMILES: *OCCN(*)C]

In embodiments, L¹ is independently unsubstituted C₁-C₃ alkylene, -L²⁸-O—, or —O-L²⁸-, and L²⁸ is independently a bond or substituted or unsubstituted C₁-C₃ alkylene. In embodiments, L¹ is independently unsubstituted C₁-C₃ alkylene. In embodiments, L¹ is independently -L²⁸-O—. In embodiments, L¹ is independently —O-L²⁸-.

In embodiments, L²⁸ is independently a bond. In embodiments, L²⁸ is independently a substituted or unsubstituted C₁-C₃ alkylene. In embodiments, L²⁸ is independently a substituted C₁-C₃ alkylene. In embodiments, L²⁸ is independently an unsubstituted C₁-C₃ alkylene. In embodiments, L²⁸ is independently a substituted or unsubstituted methylene. In embodiments, L²⁸ is independently a substituted methylene. In embodiments, L²⁸ is independently an unsubstituted methylene. In embodiments, L²⁸ is independently a substituted or unsubstituted ethylene. In embodiments, L²⁸ is independently a substituted ethylene. In embodiments, L²⁸ is independently an unsubstituted ethylene. In embodiments, L²⁸ is independently a substituted or unsubstituted propylene. In embodiments, L²⁸ is independently a substituted propylene. In embodiments, L²⁸ is independently an unsubstituted propylene.

In embodiments, L² is substituted or unsubstituted C₁-C₃ alkylene. In embodiments, L² is substituted or unsubstituted methylene. In embodiments, L² is substituted or unsubstituted C₁-C₆ alkylene, or substituted or unsubstituted 2 to 6 membered heteroalkylene. In embodiments, L² is substituted or unsubstituted C₁-C₃ alkylene, or substituted or unsubstituted 2 to 3 membered heteroalkylene.

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene. In embodiments, L² is unsubstituted alkylene (e.g., C₁-C₆ alkylene). In embodiments, L² is a bond.

In embodiments, R^{1A} is independently a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{1A} is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1A} is independently unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1A} is independently hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{1A} is independently substituted or unsubstituted alkyl (e.g., C₁-C₈ alkyl, C₁-C₆ alkyl, or C₁-C₄ alkyl). In embodiments, R^{1A} is independently substituted alkyl (e.g., C₁-C₈ alkyl, C₁-C₆ alkyl, or C₁-C₄ alkyl). In embodiments, R^{1A} is independently an unsubstituted alkyl (e.g., C₁-C₈ alkyl, C₁-C₆ alkyl, or C₁-C₄ alkyl). In embodiments, R^{1A} is independently substituted or unsubstituted heteroalkyl (e.g., 2 to 8 membered heteroalkyl, 2 to 6 membered heteroalkyl, or 2 to 4 membered heteroalkyl). In embodiments, R^{1A} is independently substituted heteroalkyl (e.g., 2 to 8 membered heteroalkyl, 2 to 6 membered heteroalkyl, or 2 to 4 membered heteroalkyl). In embodiments, R^{1A} is independently an unsubstituted heteroalkyl (e.g., 2 to 8 membered heteroalkyl, 2 to 6 membered heteroalkyl, or 2 to 4 membered heteroalkyl). In embodiments, R^{1A} is independently substituted or unsubstituted cycloalkyl (e.g., C₃-C₈ cycloalkyl, C₃-C₆ cycloalkyl, or C₅-C₆ cycloalkyl). In embodiments, R^{1A} is independently substituted cycloalkyl (e.g., C₃-C₈ cycloalkyl, C₃-C₆ cycloalkyl, or C₅-C₆ cycloalkyl). In embodiments, R^{1A} is independently an unsubstituted cycloalkyl (e.g., C₃-C₈ cycloalkyl, C₃-C₆ cycloalkyl, or C₅-C₆ cycloalkyl). In embodiments, R^{1A} is independently substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is independently substituted heterocycloalkyl (e.g., 3 to 8 membered heterocycloalkyl, 3 to 6 membered heterocycloalkyl, or 5 to 6 membered heterocycloalkyl). In embodiments, R^{1A} is independently an unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is independently substituted or unsubstituted aryl (e.g., C₆-C₁₀ aryl, C₁₀ aryl, or phenyl). In embodiments, R^{1A} is substituted aryl (e.g., C₆-C₁₀ aryl, C₁₀ aryl, or phenyl). In embodiments, R^{1A} is independently an unsubstituted aryl (e.g., C₆-C₁₀ aryl, C₁₀ aryl, or phenyl). In embodiments, R^{1A} is independently substituted or unsubstituted heteroaryl (e.g., 5 to 10 membered heteroaryl, 5 to 9 membered heteroaryl, or 5 to 6 membered heteroaryl). In embodiments, R^{1A} is independently substituted heteroaryl (e.g., 5 to 10 membered heteroaryl, 5 to 9 membered heteroaryl, or 5 to 6 membered heteroaryl). In embodiments, R^{1A} is independently an unsubstituted heteroaryl (e.g., 5 to 10 membered heteroaryl, 5 to 9 membered heteroaryl, or 5 to 6 membered heteroaryl).

In embodiments, R^{1A} is independently a substituted or unsubstituted aryl. In some other embodiments, R^{1A} is independently a substituted or unsubstituted phenyl. In still some other embodiments, R^{1A} is independently a substituted or unsubstituted aryl. In still some other embodiments, R^{1A} is independently a substituted or unsubstituted phenyl or naphthalenyl.

In embodiments, R^{2A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{2A} is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{2A} is independently unsubstituted alkyl, unsubstituted heteroalkyl, unsubstituted cycloalkyl, unsubstituted heterocycloalkyl, unsubstituted aryl, or unsubstituted heteroaryl. In embodiments, R^{2A} is independently hydrogen.

In embodiments, R^{2A} is independently hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{2A} is independently substituted or unsubstituted alkyl (e.g., C₁-C₈ alkyl, C₁-C₆ alkyl, or C₁-C₄ alkyl). In embodiments, R^{2A} is independently substituted alkyl (e.g., C₁-C₈ alkyl, C₁-C₆ alkyl, or C₁-C₄ alkyl). In embodiments, R^{2A} is independently an unsubstituted alkyl (e.g., C₁-C₈ alkyl, C₁-C₆ alkyl, or C₁-C₄ alkyl). In embodiments, R^{2A} is independently substituted or unsubstituted heteroalkyl (e.g., 2 to 8 membered heteroalkyl, 2 to 6 membered heteroalkyl, or 2 to 4 membered heteroalkyl). In embodiments, R^{2A} is independently substituted heteroalkyl (e.g., 2 to 8 membered heteroalkyl, 2 to 6 membered heteroalkyl, or 2 to 4 membered heteroalkyl). In embodiments, R^{2A} is independently an unsubstituted heteroalkyl (e.g., 2 to 8 membered heteroalkyl, 2 to 6 membered heteroalkyl, or 2 to 4 membered heteroalkyl). In embodiments, R^{2A} is independently substituted or unsubstituted cycloalkyl (e.g., C₃-C₈ cycloalkyl, C₃-C₆ cycloalkyl, or C₅-C₆ cycloalkyl). In embodiments, R^{2A} is independently substituted cycloalkyl (e.g., C₃-C₈ cycloalkyl, C₃-C₆ cycloalkyl, or C₅-C₆ cycloalkyl). In embodiments, R^{2A} is an unsubstituted cycloalkyl (e.g., C₃-C₈ cycloalkyl, C₃-C₆ cycloalkyl, or C₅-C₆ cycloalkyl). In embodiments, R^{2A} is independently substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is independently substituted heterocycloalkyl (e.g., 3 to 8 membered heterocycloalkyl, 3 to 6 membered heterocycloalkyl, or 5 to 6 membered heterocycloalkyl). In embodiments, R^{2A} is independently an unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is independently substituted or unsubstituted aryl (e.g., C₆-C₁₀ aryl, C₁₀ aryl, or phenyl). In embodiments, R^{2A} is substituted aryl (e.g., C₆-C₁₀ aryl, C₁₀ aryl, or phenyl). In embodiments, R^{2A} is independently an unsubstituted aryl (e.g., C₆-C₁₀ aryl, C₁₀ aryl, or phenyl). In embodiments, R^{2A} is independently substituted or unsubstituted heteroaryl (e.g., 5 to 10 membered heteroaryl, 5 to 9 membered heteroaryl, or 5 to 6 membered heteroaryl). In embodiments, R^{2A} is independently substituted heteroaryl (e.g., 5 to 10 membered heteroaryl, 5 to 9 membered heteroaryl, or 5 to 6 membered heteroaryl). In embodiments, R^{2A} is independently an unsubstituted heteroaryl (e.g., 5 to 10 membered heteroaryl, 5 to 9 membered heteroaryl, or 5 to 6 membered heteroaryl).

In embodiments z2 is an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25). In embodiments, z2 is 5 to 25. In embodiments, z2 is 10 to 25.

In embodiments, z1 and z3 are independently integers from 0 to 90 (e.g. 5 to 90, 10 to 90 or 20 to 90), 0 to 80 (e.g. 5 to 80, 10 to 80 or 20 to 80), 0 to 70 (e.g. 5 to 70, 10 to 70 or 20 to 70), 0 to 50 (e.g. 5 to 50, 10 to 50 or 20 to 50) or 2 to 25. In embodiments, z1 and z3 are independently integers from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25). In embodiments, z1 and z3 are independently integers from 5 to 30 (e.g., 5 to 25, or 5 to 20). In embodiments, z1 and z3 are independently integers from 5 to 30. In embodiments, z1 and z3 are independently 5 to 25. In embodiments, z1 and z3 are independently 5 to 20.

In embodiments, z4 is an integer from 1 to 100 (e.g. 5 to 100, 10 to 100 or 20 to 100), 1 to 90 (e.g. 5 to 90, 10 to 90 or 20 to 90), 1 to 80 (e.g. 5 to 80, 10 to 80 or 20 to 80), 1 to 70 (e.g. 5 to 70, 10 to 70 or 20 to 70), 1 to 50 (e.g. 5 to 50, 10 to 50 or 20 to 50) or 2 to 25. In embodiments, z4 is an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25).

In embodiments, each LP¹ or LP² is independently anon-linear branched lipophilic monomer having a structure of

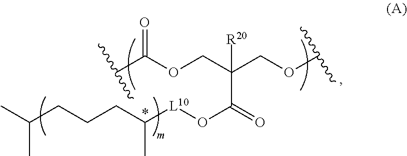

wherein
m is an integer from 0 to 6;
L¹⁰ is independently a substituted or unsubstituted alkylene; and
R²⁰ is independently hydrogen or unsubstituted C₁-C₄ alkyl.

In embodiments, L¹⁰ is independently an unsubstituted C₁-C₄ alkylene. In embodiments, L¹⁰ is independently an unsubstituted methylene. In embodiments, L¹⁰ is independently an unsubstituted ethylene. In embodiments, L¹⁰ is independently an unsubstituted propylene. In embodiments, L¹⁰ is independently an unsubstituted isopropylene. In embodiments, L¹⁰ is independently an unsubstituted butylene. In embodiments, L¹⁰ is independently an unsubstituted t-butylene.

In embodiments, m is an integer from 0 to 6 (e.g., 0 to 6, 1 to 6, 1 to 5, 1 to 4, or 1 to 3). In embodiments, m is 1, 2, or 3. In embodiments, m is 1. In embodiments, m is 2. In embodiments, m is 3.

In embodiments, each LP¹ and LP² is independently

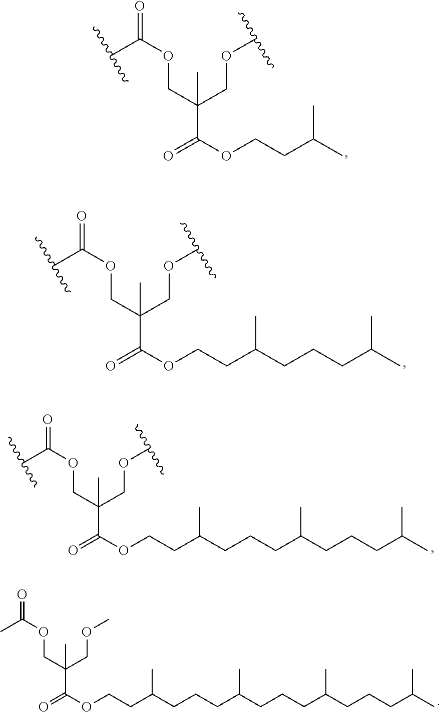

In embodiments, LP¹ is independently

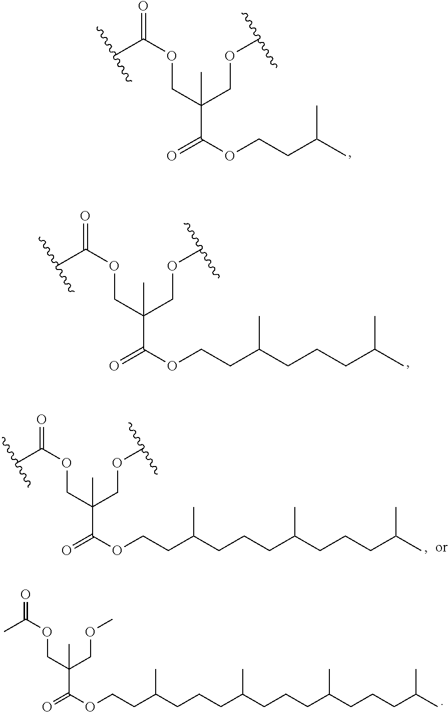

In embodiments, LP¹ is independently

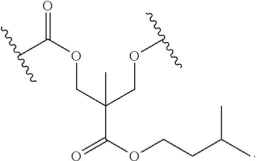 [SMILES: *OCC(C)(COC(*)=O)C(=O)OCCC(C)C]

In embodiments, LP¹ is independently

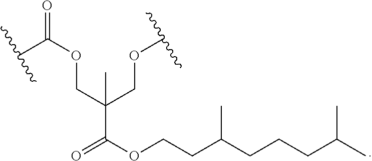 [SMILES: *OCC(C)(COC(*)=O)C(=O)OCCC(C)CCCC(C)C]

In embodiments, LP¹ is independently

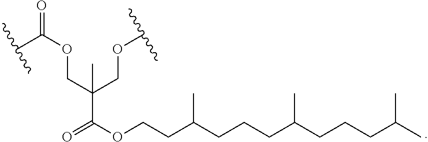 [SMILES: *OCC(C)(COC(*)=O)C(=O)OCCC(C)CCCC(C)CCCC(C)C]

In embodiments, LP¹ is independently

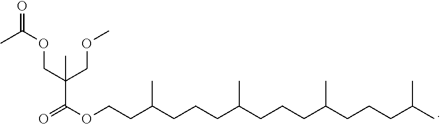 [SMILES: COCC(C)(COC(C)=O)C(=O)OCCC(C)CCCC(C)CCCC(C)CCCC(C)C]

In embodiments, LP² is independently

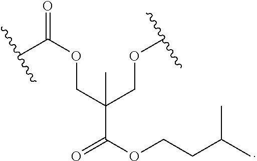 [SMILES: *OCC(C)(COC(*)=O)C(=O)OCCC(C)C]

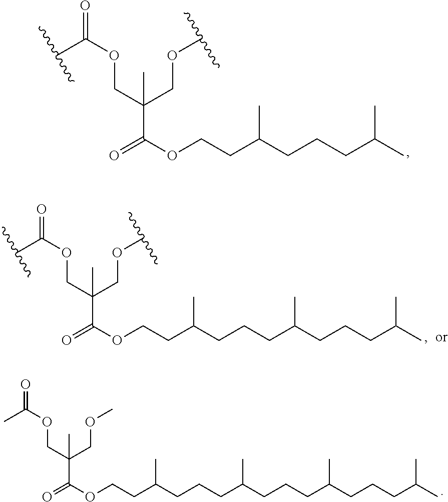

In embodiments, LP² is independently

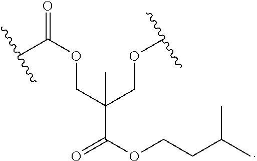 [SMILES: *OCC(C)(COC(*)=O)C(=O)OCCC(C)C]

In embodiments, LP² is independently

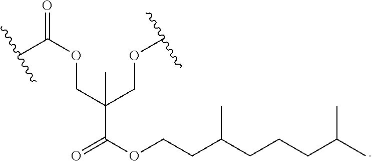 [SMILES: *OCC(C)(COC(*)=O)C(=O)OCCC(C)CCCC(C)C]

In embodiments, LP² is independently

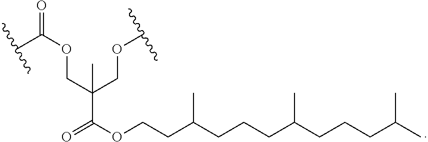 [SMILES: *OCC(C)(COC(*)=O)C(=O)OCCC(C)CCCC(C)CCCC(C)C]

In embodiments, LP² is independently

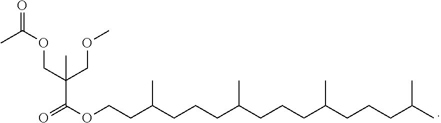 [SMILES: COCC(C)(COC(C)=O)C(=O)OCCC(C)CCCC(C)CCCC(C)CCCC(C)C]

In embodiments, each LP¹ or LP² are independently a non-linear branched lipophilic monomer having a structure of:

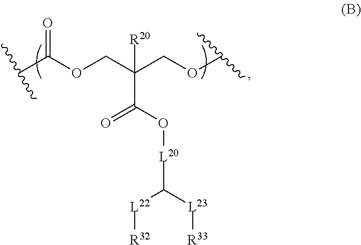

wherein
L²⁰ is -L^{20A}-L^{20B}-;
each L^{20A} and L^{20B} is independently a bond, a substituted or unsubstituted alkylene, substituted or unsubstituted heteroalkylene, substituted or unsubstituted cycloalkylene, or substituted or unsubstituted heterocycloalkylene;
each L²², and L²³ are independently a bond, substituted or unsubstituted alkylene, or substituted or unsubstituted heteroalkylene;
R²⁰ is independently hydrogen or unsubstituted C₁-C₄ alkyl; and
each R³² and R³³ are independently hydrogen, or substituted or unsubstituted alkyl.

In embodiment, L²² is independently a bond, unsubstituted C₁-C₄ alkylene, -L²⁴-OC(O)—, or -L²⁴-C(O)O—; L²³ is independently a bond, unsubstituted C₁-C₄ alkylene, -L²⁵-OC(O)—, or -L²⁵-C(O)O—; and each L²⁴ and L²⁵ is independently a bond or substituted or unsubstituted C₁-C₃ alkylene.

In embodiment, L²² is independently a bond, unsubstituted C₁-C₄ alkylene, -L²⁴-OC(O)—, or -L²⁴-C(O)O—. In embodiment, L²² is independently a bond. In embodiment, L²² is independently an unsubstituted C₁-C₄ alkylene. In embodiments, L²² is independently an unsubstituted methyl. In embodiments, L²² is independently an unsubstituted ethyl. In embodiments, L²² is independently an unsubstituted propyl. In embodiments, L²² is independently an unsubstituted isopropyl. In embodiments, L²² is independently an unsubstituted butyl. In embodiments, L²² is independently an unsubstituted t-butyl.

In embodiment, L²² is -L²⁴-OC(O)—. In embodiment, L²² is -L²⁴-C(O)O—. In embodiment, L²⁴ is independently an unsubstituted C₁-C₄ alkylene. In embodiments, L²⁴ is independently an unsubstituted methyl. In embodiments, L²⁴ is independently an unsubstituted ethyl. In embodiments, L²⁴ is independently an unsubstituted propyl. In embodiments, L²⁴ is independently an unsubstituted isopropyl. In embodiments, L²⁴ is independently an unsubstituted butyl. In embodiments, L²⁴ is independently an unsubstituted t-butyl.

In embodiment, L²³ is independently a bond, unsubstituted C₁-C₄ alkylene, -L²⁵-OC(O)—, or -L²⁵-C(O)O—. In embodiment, L²³ is independently a bond. In embodiment, L²³ is independently an unsubstituted C₁-C₄ alkylene. In embodiments, L²³ is independently an unsubstituted methyl. In embodiments, L²³ is independently an unsubstituted ethyl. In embodiments, L²³ is independently an unsubstituted propyl. In embodiments, L²³ is independently an unsubstituted isopropyl. In embodiments, L²³ is independently an unsubstituted butyl. In embodiments, L²³ is independently an unsubstituted t-butyl.

In embodiment, L²³ is -L²⁵-OC(O)—. In embodiment, L²³ is -L²⁵-C(O)O—. In embodiment, L²⁵ is independently an unsubstituted C₁-C₄ alkylene. In embodiments, L²⁵ is independently an unsubstituted methyl. In embodiments, L²⁵ is independently an unsubstituted ethyl. In embodiments, L²⁵ is independently an unsubstituted propyl. In embodiments, L²⁵ is independently an unsubstituted isopropyl. In embodiments, L²⁵ is independently an unsubstituted butyl. In embodiments, L²⁵ is independently an unsubstituted t-butyl.

In embodiments, L^{20A} is independently a bond. In embodiments, L^{20B} is independently a bond. In embodiments, L^{21A} is independently a bond and L^{21B} is not a bond. In embodiments, L^{20B} is independently a bond and L^{20A} is not a bond. In embodiments, L^{20A} is independently a bond and L^{20B} is substituted or unsubstituted alkylene, or substituted or unsubstituted heteroalkylene. In embodiments, L^{20A} is independently a bond and L^{20B} is substituted or unsubstituted alkylene. In embodiments, L^{20A} is independently a bond and L^{20B} is substituted or unsubstituted heteroalkylene. In embodiments, L^{20B} is independently a bond and L^{20A} is substituted or unsubstituted alkylene, or substituted or unsubstituted heteroalkylene. In embodiments, L^{20B} is independently a bond and L^{20A} is substituted or unsubstituted alkylene. In embodiments, L^{20B} is independently a bond and L^{20A} is substituted or unsubstituted heteroalkylene. In embodiments, L^{20A} is independently a bond and L^{20B} is independently a bond.

In embodiments, L²⁰ is independently a bond. In embodiments, L²⁰ is independently unsubstituted C₁-C₁₀ alkylene, -L^{21A}-O—, —O-L^{21B}-, -L^{21A}-O-L^{21B}-, -L^{21A}-C(O)—O—, —C(O)O-L^{21B}-, -L^{21A}-C(O)O-L^{21B}-, -L^{21A}-OC(O)—, —OC(O)-L^{21B}-, -L^{21A}-OC(O)-L^{21B}-, -L^{21A}-O—C(O)—O—, —OC(O)O-L^{21B}-, or -L^{21A}-OC(O)O-L^{21B}-, and each L^{21A} and L^{21B} is independently a bond or substituted or unsubstituted C₁-C₁₀ alkylene.

In embodiments, L²⁰ is independently unsubstituted C₁-C₁₀ alkylene. In embodiments, L²⁰ is independently unsubstituted C₂-C₁₀ alkylene. In embodiments, L²⁰ is independently unsubstituted C₃-C₁₀ alkylene. In embodiments, L²⁰ is independently unsubstituted C₅-C₁₀ alkylene. In embodiments, L²⁰ is independently unsubstituted C₁-C₈alkylene. In embodiments, L²⁰ is independently unsubstituted C₁-C₆ alkylene. In embodiments, L²⁰ is independently unsubstituted C₂-C₈ alkylene. In embodiments, L²⁰ is independently unsubstituted C₄-C₈ alkylene. In embodiments, L²⁰ is independently unsubstituted C₅-C₈ alkylene. In embodiments, L²⁰ is independently unsubstituted C₅-C₆ linear alkylene. In embodiments, L²⁰ is independently unsubstituted C₁-C₁₀ linear alkylene. In embodiments, L²⁰ is independently unsubstituted C₂-C₁₀ linear alkylene. In embodiments, L²⁰ is independently unsubstituted C₃-C₁₀ linear alkylene. In embodiments, L²⁰ is independently unsubstituted C₅-C₁₀ linear alkylene. In embodiments, L²⁰ is independently unsubstituted C₁-C₈ linear alkylene. In embodiments, L²⁰ is independently unsubstituted C₁-C₆ linear alkylene. In embodiments, L²⁰ is independently unsubstituted C₂-C₈ linear alkylene. In embodiments, L²⁰ is independently unsubstituted C₄-C₈ linear alkylene. In embodiments, L²⁰ is independently unsubstituted C₅-C₈ linear alkylene. In embodiments, L²⁰ is independently unsubstituted C₅-C₆ linear alkylene. In embodiments, L²⁰ is independently unsubstituted C₅-C₆ branched alkylene. In embodiments, L²⁰ is independently unsubstituted C₁-C₁₀ branched alkylene. In embodiments, L²⁰ is independently unsubstituted C₂-C₁₀ branched alkylene. In embodiments, L²⁰ is independently unsubstituted C₃-C₁₀ branched alkylene. In embodiments, L²⁰ is independently unsubstituted C₅-C₁₀ branched alkylene. In embodiments, L²⁰ is independently unsubstituted C₁-C₈ branched alkylene. In embodiments, L²⁰ is independently unsubstituted C₁-C₆ branched alkylene. In embodiments, L²⁰ is independently unsubstituted C₂-C₈ branched alkylene. In embodiments, L²⁰ is independently unsubstituted C₄-C₈ branched alkylene. In embodiments, L²⁰ is independently unsubstituted C₅-C₈ branched alkylene. In embodiments, L²⁰ is independently unsubstituted C₅-C₆ branched alkylene.

In embodiments, L²⁰ is independently -L^{21A}-O—, —O-L^{21B}-, or -L^{21A}-O-L^{21B}-. In embodiments, L²⁰ is independently -L^{21A}-O—. In embodiments, L²⁰ is independently —O-L^{21B}-. In embodiments, L²⁰ is independently -L^{21A}-O-L^{21B}-. In embodiments, L²⁰ is independently -L^{21A}-C(O)—O—, —C(O)O-L^{21B}-, or -L^{21A}-C(O)O-L^{21B}-. In embodiments, L²⁰ is independently -L^{21A}-C(O)—O—. In embodiments, L²⁰ is independently —C(O)O-L^{21B}-. In embodiments, L²⁰ is independently -L^{21A}-C(O)O-L^{21B}-. In embodiments, L²⁰ is independently -L^{21A}-OC(O)—, —OC(O)-L^{21B}-, or -L^{21A}-OC(O)-L^{21B}-. In embodiments, L²⁰ is independently -L^{21A}-OC(O)—. In embodiments, L²⁰ is independently —OC(O)-L^{21B}-. In embodiments, L²⁰ is independently -L^{21A}-OC(O)-L^{21B}-. In embodiments, L²⁰ is independently -L^{21A}-O—C(O)—O—, —O—C(O)—O-L^{21B}-, or -L^{21A}-O—C(O)—O-L^{21B}-. In embodiments, L²⁰ is independently -L^{21A}-O—C(O)—O—. In embodiments, L²⁰ is independently —O—C(O)—O-L^{21B}-. In embodiments, L²⁰ is independently -L^{21A}-O—C(O)—O-L^{21B}-.

In embodiments, L^{21A} is independently unsubstituted C₁-C₁₀ alkylene. In embodiments, L^{21A} is independently unsubstituted C₂-C₁₀ alkylene. In embodiments, L^{21A} is independently unsubstituted C₃-C₁₀ alkylene. In embodiments, L^{21A} is independently unsubstituted C₅-C₁₀ alkylene. In embodiments, L^{21A} is independently unsubstituted C₁-C₈ alkylene. In embodiments, L^{21A} is independently unsubstituted C₁-C₆ alkylene. In embodiments, L^{21A} is independently unsubstituted C₂-C₈ alkylene. In embodiments, L^{21A} is independently unsubstituted C₄-C₈ alkylene. In embodiments, L^{21A} is independently unsubstituted C₅-C₈ alkylene. In embodiments, L^{21A} is independently unsubstituted C₅-C₆ linear alkylene. In embodiments, L^{21A} is independently unsubstituted C₁-C₁₀ linear alkylene. In embodiments, L^{21A} is independently unsubstituted C₂-C₁₀ linear alkylene. In embodiments, L^{21A} is independently unsubstituted C₃-C₁₀ linear alkylene. In embodiments, L^{21A} is independently unsubstituted C₅-C₁₀ linear alkylene. In embodiments, L²¹a is independently unsubstituted C₁-C₈ linear alkylene. In embodiments, L^{21A} is independently unsubstituted C₁-C₆ linear alkylene. In embodiments, L^{21A} is independently unsubstituted C₂-C₈ linear alkylene. In embodiments, L^{21A} is independently unsubstituted C₄-C₈ linear alkylene. In embodiments, L^{21A} is independently unsubstituted C₅-C₈ linear alkylene. In embodiments, L^{21A} is independently unsubstituted C₅-C₆ linear alkylene. In embodiments, L^{21A} is independently unsubstituted C₅-C₆ branched alkylene. In embodiments, L^{21A} is independently unsubstituted C₁-C₁₀ branched alkylene. In embodiments, L^{21A} is independently unsubstituted C₂-C₁₀ branched alkylene. In embodiments, L^{21A} is independently unsubstituted C₃-C₁₀ branched alkylene. In embodiments, L^{21A} is independently unsubstituted C₅-C₁₀ branched alkylene. In embodiments, L^{21A} is independently unsubstituted C₁-C₈ branched alkylene. In embodiments, L^{21A} is independently unsubstituted C₁-C₆ branched alkylene. In embodiments, L^{21A} is independently unsubstituted C₂-C₈ branched alkylene. In embodiments, L^{21A} is independently unsubstituted C₄-C₈ branched alkylene. In embodiments, L^{21A} is independently unsubstituted C₅-C₈ branched alkylene. In embodiments, L^{21A} is independently unsubstituted C₅-C₆ branched alkylene.

In embodiments, L^{21B} is independently unsubstituted C₁-C₁₀ alkylene. In embodiments, L^{21B} is independently unsubstituted C₂-C₁₀ alkylene. In embodiments, L^{21B} is independently unsubstituted C₃-C₁₀ alkylene. In embodiments, L^{21B} is independently unsubstituted C₅-C₁₀ alkylene. In embodiments, L^{21B} is independently unsubstituted C₁-C₈ alkylene. In embodiments, L^{21B} is independently unsubstituted C₁-C₆ alkylene. In embodiments, L^{21B} is independently unsubstituted C₁-C₄ alkylene. In embodiments, L^{21B} is independently unsubstituted C₁-C₃ alkylene. In embodiments, L^{21B} is independently unsubstituted C₂-C₈ alkylene. In embodiments, L^{21B} is independently unsubstituted C₄-C₈ alkylene. In embodiments, L^{21B} is independently unsubstituted C₅-C₈ alkylene. In embodiments, L^{21B} is independently unsubstituted C₅-C₆ linear alkylene. In embodiments, L^{21B} is independently unsubstituted C₁-C₁₀ linear alkylene. In embodiments, L^{21B} is independently unsubstituted C₂-C₁₀ linear alkylene. In embodiments, L^{21B} is independently unsubstituted C₃-C₁₀ linear alkylene. In embodiments, L^{21B} is independently unsubstituted C₅-C₁₀ linear alkylene. In embodiments, L^{21B} is independently unsubstituted C₁-C₈ linear alkylene. In embodiments, L^{21B} is independently unsubstituted C₁-C₆ linear alkylene. In embodiments, L^{21B} is independently unsubstituted linear C₁-C₄ alkylene. In embodiments, L^{21B} is independently unsubstituted linear C₁-C₃ alkylene. In embodiments, L^{21B} is independently unsubstituted C₂-C₈ linear alkylene. In embodiments, L^{21B} is independently unsubstituted C₄-C₈ linear alkylene. In embodiments, L^{21B} is independently unsubstituted C₅-C₈ linear alkylene. In embodiments, L^{21B} is independently unsubstituted C₅-C₆ linear alkylene. In embodiments, L^{21B} is independently unsubstituted C₅-C₆ branched alkylene. In embodiments, L^{21B} is independently unsubstituted C₁-C₁₀ branched alkylene. In embodiments, L^{21B} is independently unsubstituted C₂-C₁₀ branched alkylene. In embodiments, L^{21B} is independently unsubstituted C₃-C₁₀ branched alkylene. In embodiments, L^{21B} is independently unsubstituted C₅-C₁₀ branched alkylene. In embodiments, L^{21B} is independently unsubstituted C₁-C₈ branched alkylene. In embodiments, L^{21B} is independently unsubstituted C₁-C₆ branched alkylene. In embodiments, L^{21B} is independently unsubstituted branched C₁-C₄ alkylene. In embodiments, L^{21B} is independently unsubstituted branched C₁-C₃ alkylene. In embodiments, L^{21B} is independently unsubstituted C₂-C₈ branched alkylene. In embodiments, L^{21B} is independently unsubstituted C₄-C₈ branched alkylene. In embodiments, L^{21B} is independently unsubstituted C₅-C₈ branched alkylene. In embodiments, L^{21B} is independently unsubstituted C₅-C₆ branched alkylene.

In embodiments, each LP¹ and LP² has a structure of:

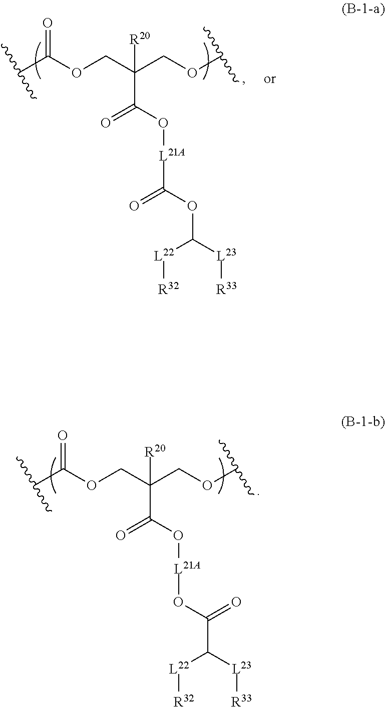

R²⁰, R³², R³³, L^{21A}, L²² and L²³ are as described herein.

In embodiments, L^{21A} is independently unsubstituted C₅-C₁₀ alkylene. In embodiments, L^{21A} is independently unsubstituted C₅-C₈ alkylene. In embodiments, L^{21A} is independently unsubstituted C₅-C₆ alkylene. In embodiments, L^{21A} is independently unsubstituted C₅-C₁₀ linear alkylene. In embodiments, L^{21A} is independently unsubstituted C₅-C₈ linear alkylene. In embodiments, L^{21A} is independently unsubstituted linear C₅-C₆ alkylene.

In embodiments, each LP¹ and LP² has a structure of:

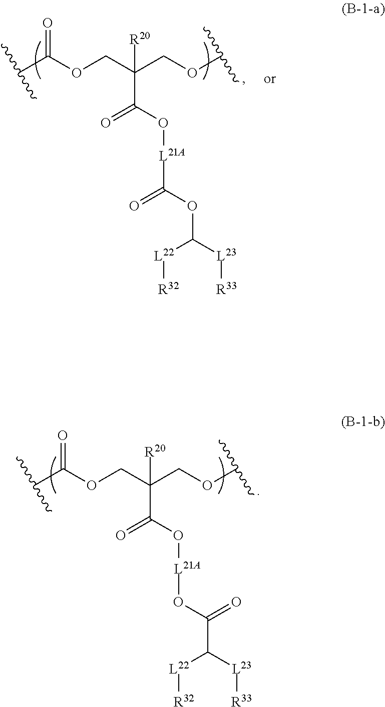

R²⁰, R³², R³³, L²¹, L^{21B} L²² and L²³ are as described herein.

In embodiments, L^{21A} is independently unsubstituted C₅-C₁₀ alkylene; and L^{21B} is independently a bond or unsubstituted C₁-C₃ alkylene.

In embodiments, L^{21A} is independently unsubstituted C₅-C₁₀ alkylene. In embodiments, L^{21A} is independently unsubstituted C₅-C₈ alkylene. In embodiments, L^{21A} is independently unsubstituted C₅-C₆ alkylene. In embodiments, L^{21A} is independently unsubstituted C₅-C₁₀ linear alkylene. In embodiments, L^{21A} is independently unsubstituted C₅-C₈ linear alkylene. In embodiments, L^{21A} is independently unsubstituted linear C₅-C₆ alkylene.

In embodiments, L^{21B} is independently a bond or unsubstituted C₁-C₃ alkylene. In embodiments, L^{21B} is independently a bond. In embodiments, L^{21B} is independently unsubstituted methylene. In embodiments, L^{21B} is independently unsubstituted ethylene. In embodiments, L^{21B} is independently unsubstituted propylene.

In embodiments, each LP¹ and LP² independently has a structure of

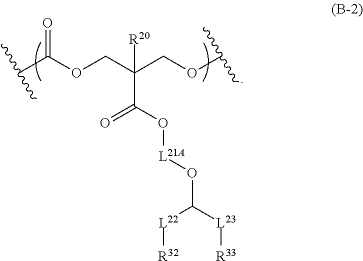

R²⁰, R³², R³³, L^{21A}, L²² and L²³ are as described herein. In embodiments, L^{21A} is independently unsubstituted C₅-C₁₀ alkylene.

In embodiments, each LP¹ and LP² independently has a structure of

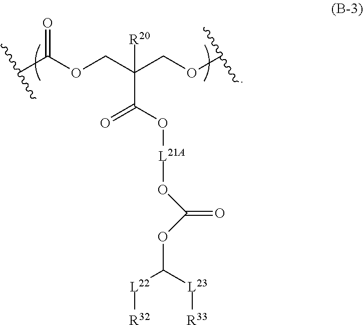

R²⁰, R³², R³³, L^{21A}, L²² and L²³ are as described herein. In embodiments, L^{21A} is independently unsubstituted C₅-C₁₀ alkylene.

In embodiments, L²² is independently a bond, or unsubstituted C₁-C₄ alkylene.

In embodiments, L²² is independently a bond or unsubstituted C₁-C₄ alkylene. In embodiments, L²² is independently a bond. In embodiments, L²² is independently unsubstituted methylene. In embodiments, L²² is independently unsubstituted ethylene. In embodiments, L²² is independently unsubstituted propylene. In embodiments, L²² is independently unsubstituted butylene.

In embodiments, L²³ is independently a bond or unsubstituted C₁-C₄ alkylene. In embodiments, L²³ is independently a bond. In embodiments, L²³ is independently unsubstituted methylene. In embodiments, L²³ is independently unsubstituted ethylene. In embodiments, L²³ is independently unsubstituted propylene. In embodiments, L²³ is independently unsubstituted butylene.

In embodiments, each LP¹ and LP² independently has a structure of

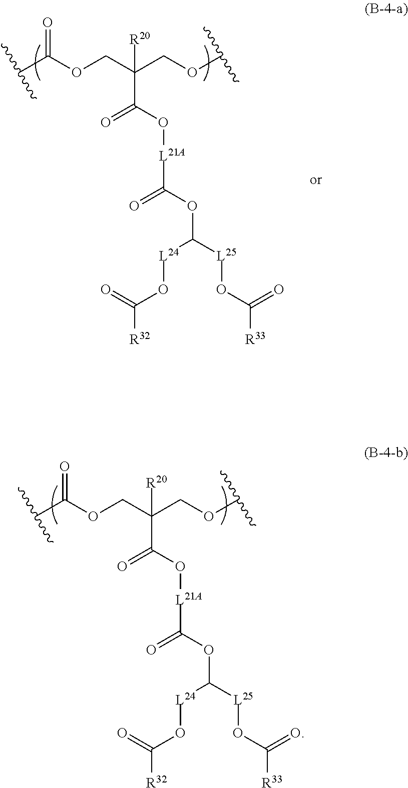

R²⁰, R³², R³³, L^{21A}, L²² and L²³ are as described herein. In embodiments, L^{21A} is independently unsubstituted C₅-C₁₀ alkylene.

In embodiments, each LP¹ and LP² has a structure of:

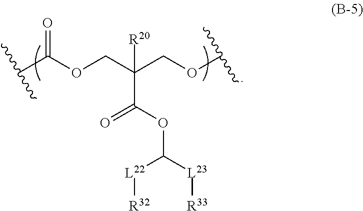

R²⁰, R³², R³³, L²² and L²³ are as described herein.

In embodiments, L²² is independently a bond. In embodiments, L²³ is independently a bond.

In embodiments, each LP¹ and LP² is independently

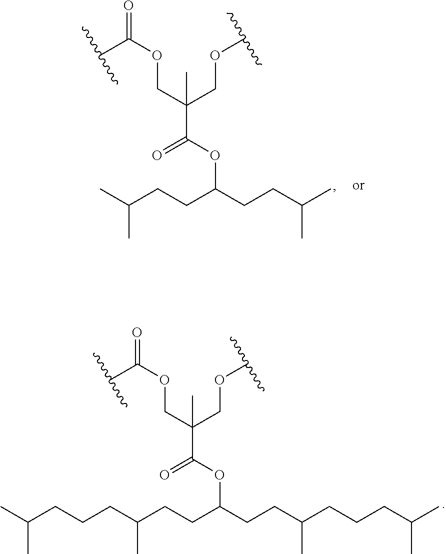 [SMILES: *OCC(C)(COC(*)=O)C(=O)OC(CCC(C)C)CCC(C)C ; *OCC(C)(COC(*)=O)C(=O)OC(CCC(C)CCCC(C)C)CCC(C)CCCC(C)C]

In embodiments, LP¹ is independently

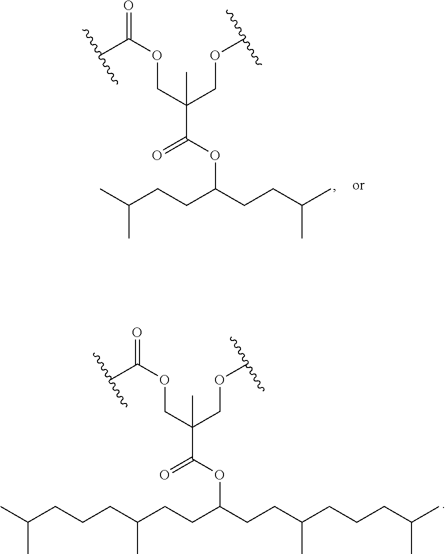 [SMILES: *OCC(C)(COC(*)=O)C(=O)OC(CCC(C)C)CCC(C)C ; *OCC(C)(COC(*)=O)C(=O)OC(CCC(C)CCCC(C)C)CCC(C)CCCC(C)C]

In embodiments, LP² is independently

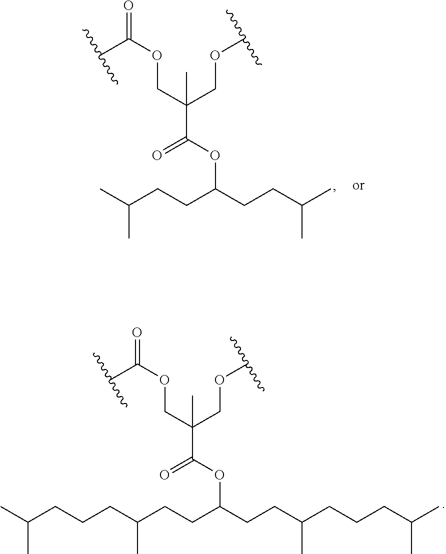 [SMILES: *OCC(C)(COC(*)=O)C(=O)OC(CCC(C)C)CCC(C)C ; *OCC(C)(COC(*)=O)C(=O)OC(CCC(C)CCCC(C)C)CCC(C)CCCC(C)C]

In embodiments, L^{20A} is not a bond. In embodiments, L^{20B} is not a bond. In embodiments, L^{20A} is substituted or unsubstituted cycloalkylene, or substituted or unsubstituted heterocycloalkylene. In embodiments, L^{20B} is a substituted or unsubstituted alkylene, or substituted or unsubstituted heteroalkylene. In embodiments, L^{20A} is substituted or unsubstituted cycloalkylene, or substituted or unsubstituted heterocycloalkylene, and L^{20B} is a substituted or unsubstituted alkylene. In embodiments, L^{20A} is substituted or unsubstituted C₃-C₆ cycloalkylene, or substituted or unsubstituted 4 to 6 membered heterocycloalkylene, and L^{20B} is a substituted or unsubstituted C₁-C₄ alkylene. In embodiments, L^{20A} is substituted or unsubstituted cycloalkylene, or substituted or unsubstituted heterocycloalkylene, and L^{20B} is substituted or unsubstituted heteroalkylene. In embodiments, L^{20A} is substituted or unsubstituted C₃-C₆ cycloalkylene, or substituted or unsubstituted 4 to 6 membered heterocycloalkylene, and L^{20B} is a substituted or unsubstituted 2 to 5 membered heteroalkylene.

In embodiments, L^{20A} is substituted or unsubstituted C₃-C₆ cycloalkylene, or substituted or unsubstituted 4 to 6 membered heterocycloalkylene, and L^{20B} is —O—C(O)—O—. In embodiments, L^{20A} is substituted or unsubstituted C₃-C₆ cycloalkylene and L^{20B} is —O—C(O)—O—. In embodiments, L^{20A} is substituted or unsubstituted 4 to 6 membered heterocycloalkylene, and L^{20B} is —O—C(O)—O—.

In embodiments, each LP¹ and LP² has a structure of

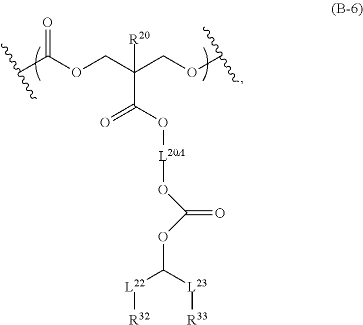

wherein L^{20A} is substituted or unsubstituted cycloalkylene, or substituted or unsubstituted heterocycloalkylene. R²⁰, R³², R³³, L²² and L²³ are as described herein.

In embodiments, each LP¹ and LP² is independently

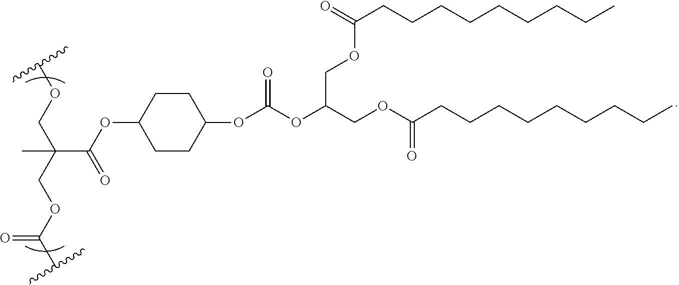

In embodiments, LP¹ is independently

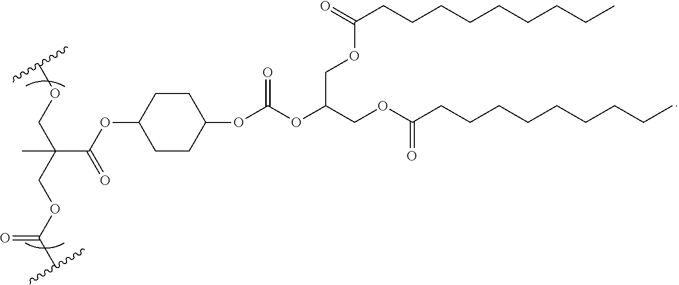

In embodiments, LP² is independently

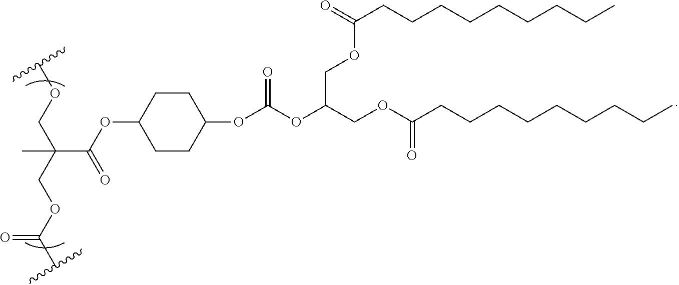

In embodiments, R²⁰ is independently C₁-C₄ alkyl. In embodiments, R²⁰ is independently methyl. In embodiments, R²⁰ is independently ethyl. In embodiments, each R²⁰ is independently propyl. In embodiments, R²⁰ is independently isopropyl. In embodiments, R²⁰ is independently butyl. In embodiments, R²⁰ is independently t-butyl. In embodiments, R²⁰ is independently hydrogen.

In embodiments, each R³² and R³³ is independently unsubstituted C₄-C₂₀ alkyl. In embodiments, each R³² and R³³ is independently unsubstituted saturated C₄-C₂₀ alkyl. In embodiments, each R³² and R³³ is independently unsubstituted unsaturated C₄-C₂₀ alkyl.

In embodiments, R³² is independently unsubstituted C₄-C₂₀ alkyl. In embodiments, R³² is independently unsubstituted C₅-C₂₀ alkyl. In embodiments, R³² is independently unsubstituted C₁₀-C₂₀ alkyl. In embodiments, R³² is independently unsubstituted C₁₂-C₂₀ alkyl. In embodiments, R³² is independently unsubstituted C₄-C₁₈ alkyl. In embodiments, R³² is independently unsubstituted C₄-C₁₆ alkyl. In embodiments, R³² is independently unsubstituted C₄-C₁₄ alkyl.

In embodiments, R³² is independently unsubstituted saturated C₄-C₂ alkyl. In embodiments, R³² is independently unsubstituted saturated C₅-C₂₀ alkyl. In embodiments, R³² is independently unsubstituted saturated C₁₀-C₂₀ alkyl. In embodiments, R³² is independently unsubstituted saturated C₁₂-C₂₀ alkyl. In embodiments, R³² is independently unsubstituted saturated C₄-C₁₈ alkyl. In embodiments, R³² is independently unsubstituted saturated C₄-C₁₆ alkyl. In embodiments, R³² is independently unsubstituted saturated C₄-C₁₄ alkyl.

In embodiments, R³² is independently unsubstituted unsaturated C₄-C₂₀ alkyl. In embodiments, R³² is independently unsubstituted unsaturated C₅-C₂₀ alkyl. In embodiments, R³² is independently unsubstituted unsaturated C₁₀-C₂₀ alkyl. In embodiments, R³² is independently unsubstituted unsaturated C₁₂-C₂₀ alkyl. In embodiments, R³² is independently unsubstituted unsaturated C₄-C₁₈ alkyl. In embodiments, R³² is independently unsubstituted unsaturated C₄-C₁₆ alkyl. In embodiments, R³² is independently unsubstituted unsaturated C₄-C₁₄ alkyl.

In embodiments, R³³ is independently unsubstituted C₄-C₂₀ alkyl. In embodiments, R³³ is independently unsubstituted C₈-C₂₀ alkyl. In embodiments, R³³ is independently unsubstituted C₁₀-C₂₀ alkyl. In embodiments, R³³ is independently unsubstituted C₁₂-C₂₀ alkyl. In embodiments, R³³ is independently unsubstituted C₄-C₁₈ alkyl. In embodiments, R³³ is independently unsubstituted C₄-C₁₆ alkyl. In embodiments, R³³ is independently unsubstituted C₄-C₁₄ alkyl.

In embodiments, R³³ is independently unsubstituted saturated C₄-C₂₀ alkyl. In embodiments, R³³ is independently unsubstituted saturated C₅-C₂₀ alkyl. In embodiments, R³³ is independently unsubstituted saturated C₁₀-C₂₀ alkyl. In embodiments, R³³ is independently unsubstituted saturated C₁₂-C₂₀ alkyl. In embodiments, R³³ is independently unsubstituted saturated C₄-C₁₈ alkyl. In embodiments, R³³ is independently unsubstituted saturated C₄-C₁₆ alkyl. In embodiments, R³³ is independently unsubstituted saturated C₄-C₁₄ alkyl.

In embodiments, R³³ is independently unsubstituted unsaturated C₄-C₂₀ alkyl. In embodiments, R³³ is independently unsubstituted unsaturated C₅-C₂₀ alkyl. In embodiments, R³³ is independently unsubstituted unsaturated C₁₀-C₂₀ alkyl. In embodiments, R³³ is independently unsubstituted unsaturated C₁₂-C₂₀ alkyl. In embodiments, R³³ is independently unsubstituted unsaturated C₄-C₁R alkyl. In embodiments, R³³ is independently unsubstituted unsaturated C₄-C₁₆ alkyl. In embodiments, R³³ is independently unsubstituted unsaturated C₄-C₁₄ alkyl.

In embodiments, each R³² and R³³ is independently unsubstituted C₄-C₂₀ linear alkyl. In embodiments, each R³² and R³³ is independently unsubstituted saturated C₄-C₂₀ linear alkyl. In embodiments, each R³² and R³³ is independently unsubstituted unsaturated C₄-C₂₀ linear alkyl.

In embodiments, R³² is independently unsubstituted C₄-C₂₀ linear alkyl. In embodiments, R³² is independently unsubstituted C₈-C₂₀ linear alkyl. In embodiments, R³² is independently unsubstituted C₁₀-C₂₀ linear alkyl. In embodiments, R³² is independently unsubstituted C₁₂-C₂₀ linear alkyl. In embodiments, R³² is independently unsubstituted C₄-Cis linear alkyl. In embodiments, R³² is independently unsubstituted C₄-C₁₆ linear alkyl. In embodiments, R³² is independently unsubstituted C₄-C₁₄ linear alkyl.

In embodiments, R³² is independently unsubstituted saturated C₄-C₂₀ linear alkyl. In embodiments, R³² is independently unsubstituted saturated C₅-C₂₀ linear alkyl. In embodiments, R³² is independently unsubstituted saturated C₁₀-C₂₀ linear alkyl. In embodiments, R³² is independently unsubstituted saturated C₁₂-C₂₀ linear alkyl. In embodiments, R³² is independently unsubstituted saturated C₄-C₁₈ linear alkyl. In embodiments, R³² is independently unsubstituted saturated C₄-C₁ linear alkyl. In embodiments, R³² is independently unsubstituted saturated C₄-C₁₄ linear alkyl.

In embodiments, R³² is independently unsubstituted unsaturated C₄-C₂₀ linear alkyl. In embodiments, R³² is independently unsubstituted unsaturated C₅-C₂₀ linear alkyl. In embodiments, R³² is independently unsubstituted unsaturated C₁₀-C₂₀ linear alkyl. In embodiments, R³² is independently unsubstituted unsaturated C₁₂-C₂₀ linear alkyl. In embodiments, R³² is independently unsubstituted unsaturated C₄-C₁₈ linear alkyl. In embodiments, R³² is independently unsubstituted unsaturated C₄-C₁₆ linear alkyl. In embodiments, R³² is independently unsubstituted unsaturated C₄-C₁₄ linear alkyl.

In embodiments, R³³ is independently unsubstituted C₄-C₂₀ linear alkyl. In embodiments, R³³ is independently unsubstituted C₅-C₂₀ linear alkyl. In embodiments, R³³ is independently unsubstituted C₁₀-C₂₀ linear alkyl. In embodiments, R³³ is independently unsubstituted C₁₂-C₂₀ linear alkyl. In embodiments, R³³ is independently unsubstituted C₄-Cis linear alkyl. In embodiments, R³³ is independently unsubstituted C₄-C₁₈ linear alkyl. In embodiments, R³³ is independently unsubstituted C₄-C₁₄ linear alkyl.

In embodiments, R³ is independently unsubstituted saturated C₄-C₂₀ linear alkyl. In embodiments, R³³ is independently unsubstituted saturated C₈-C₂₀ linear alkyl. In embodiments, R³³ is independently unsubstituted saturated C₁₀-C₂₀ linear alkyl. In embodiments, R³³ is independently unsubstituted saturated C₁₂-C₂₀ linear alkyl. In embodiments, R³³ is independently unsubstituted saturated C₄-C₁₈ linear alkyl. In embodiments, R³³ is independently unsubstituted saturated C₄-C₁₆ linear alkyl. In embodiments, R³³ is independently unsubstituted saturated C₄-C₁₄ linear alkyl.

In embodiments, R³³ is independently unsubstituted unsaturated C₄-C₂₀ linear alkyl. In embodiments, R³³ is independently unsubstituted unsaturated C₈-C₂₀ linear alkyl. In embodiments, R³³ is independently unsubstituted unsaturated C₁₀-C₂₀ linear alkyl. In embodiments, R³³ is independently unsubstituted unsaturated C₁₂-C₂₀ linear alkyl. In embodiments, R³³ is independently unsubstituted unsaturated C₄-C₁₈ linear alkyl. In embodiments, R³³ is independently unsubstituted unsaturated C₄-C₁₆ linear alkyl. In embodiments, R³³ is independently unsubstituted unsaturated C₄-C₁₄ linear alkyl.

In embodiments, each R³² and R³³ is independently unsubstituted C₄-C₂₀ branched alkyl. In embodiments, each R³² and R³³ is independently unsubstituted saturated C₄-C₂₀ branched alkyl. In embodiments, each R³² and R³³ is independently unsubstituted unsaturated C₄-C₂₀ branched alkyl.

In embodiments, R³² is independently unsubstituted C₄-C₂₀ branched alkyl. In embodiments, R³² is independently unsubstituted C₅-C₂₀ branched alkyl. In embodiments, R³² is independently unsubstituted C₁₀-C₂₀ branched alkyl. In embodiments, R³² is independently unsubstituted C₁₂-C₂₀ branched alkyl. In embodiments, R³² is independently unsubstituted C₄-C₁₈ branched alkyl. In embodiments, R³² is independently unsubstituted C₄-C₁₆ branched alkyl. In embodiments, R³² is independently unsubstituted C₄-C₁₄ branched alkyl.

In embodiments, R³² is independently unsubstituted saturated C₄-C₂₀ branched alkyl. In embodiments, R³² is independently unsubstituted saturated C₈-C₂₀ branched alkyl. In embodiments, R³² is independently unsubstituted saturated C₁₀-C₂₀ branched alkyl. In embodiments, R³² is independently unsubstituted saturated C₁₂-C₂₀ branched alkyl. In embodiments, R³² is independently unsubstituted saturated C₄-C₁₈ branched alkyl. In embodiments, R³² is independently unsubstituted saturated C₄-C₁₆ branched alkyl. In embodiments, R³² is independently unsubstituted saturated C₄-C₁₄ branched alkyl.

In embodiments, R³² is independently unsubstituted unsaturated C₄-C₂₀ branched alkyl. In embodiments, R³² is independently unsubstituted unsaturated C₈-C₂₀ branched alkyl. In embodiments, R³² is independently unsubstituted unsaturated C₁₀-C₂₀ branched alkyl. In embodiments, R³² is independently unsubstituted unsaturated C₁₂-C₂₀ branched alkyl. In embodiments, R³² is independently unsubstituted unsaturated C₄-C₁₈ branched alkyl. In embodiments, R³² is independently unsubstituted unsaturated C₄-C₁₆ branched alkyl. In embodiments, R³² is independently unsubstituted unsaturated C₄-C₁₄ branched alkyl.

In embodiments, R³³ is independently unsubstituted C₄-C₂₀ branched alkyl. In embodiments, R³³ is independently unsubstituted C₈-C₂₀ branched alkyl. In embodiments, R³³ is independently unsubstituted C₁₀-C₂₀ branched alkyl. In embodiments, R³³ is independently unsubstituted C₁₂-C₂₀ branched alkyl. In embodiments, R³³ is independently unsubstituted C₄-C₁₈ branched alkyl. In embodiments, R³³ is independently unsubstituted C₄-C₁₆ branched alkyl. In embodiments, R³³ is independently unsubstituted C₄-C₁₄ branched alkyl.

In embodiments, R³³ is independently unsubstituted saturated C₄-C₂₀ branched alkyl. In embodiments, R³³ is independently unsubstituted saturated C₈-C₂₀ branched alkyl. In embodiments, R³³ is independently unsubstituted saturated C₁₀-C₂₀ branched alkyl. In embodiments, R³³ is independently unsubstituted saturated C₁₂-C₂₀ branched alkyl. In embodiments, R³³ is independently unsubstituted saturated C₄-C₁₈ branched alkyl. In embodiments, R³³ is independently unsubstituted saturated C₄-C₁₆ branched alkyl. In embodiments, R³³ is independently unsubstituted saturated C₄-C₁₄ branched alkyl.

In embodiments, R³³ is independently unsubstituted unsaturated C₄-C₂₀ branched alkyl. In embodiments, R³³ is independently unsubstituted unsaturated C₈-C₂₀ branched alkyl. In embodiments, R³³ is independently unsubstituted unsaturated C₁₀-C₂₀ branched alkyl. In embodiments, R³³ is independently unsubstituted unsaturated C₁₂-C₂₀ branched alkyl. In embodiments, R³³ is independently unsubstituted unsaturated C₄-C₁₈ branched alkyl. In embodiments, R³³ is independently unsubstituted unsaturated C₄-C₁₆ branched alkyl. In embodiments, R³³ is independently unsubstituted unsaturated C₄-C₁₄ branched alkyl.

In embodiments, each LP¹ and LP² is independently

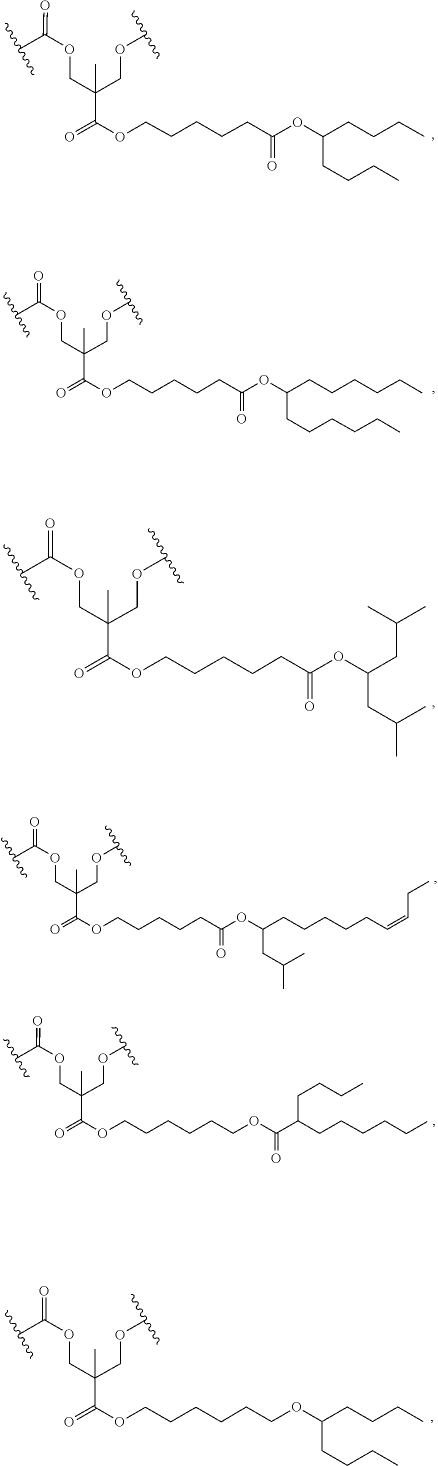 [SMILES: *OCC(C)(COC(*)=O)C(=O)OCCCCCC(=O)OC(CCCC)CCCC ; *OCC(C)(COC(*)=O)C(=O)OCCCCCC(=O)OC(CCCCCC)CCCCCC ; *OCC(C)(COC(*)=O)C(=O)OCCCCCC(=O)OC(CC(C)C)CC(C)C ; *OCC(C)(COC(*)=O)C(=O)OCCCCCC(=O)OC(CCCCC/C=C\CC)CC(C)C ; *OCC(C)(COC(*)=O)C(=O)OCCCCCCOC(=O)C(CCCC)CCCCCC ; *OCC(C)(COC(*)=O)C(=O)OCCCCCCOC(CCCC)CCCC]

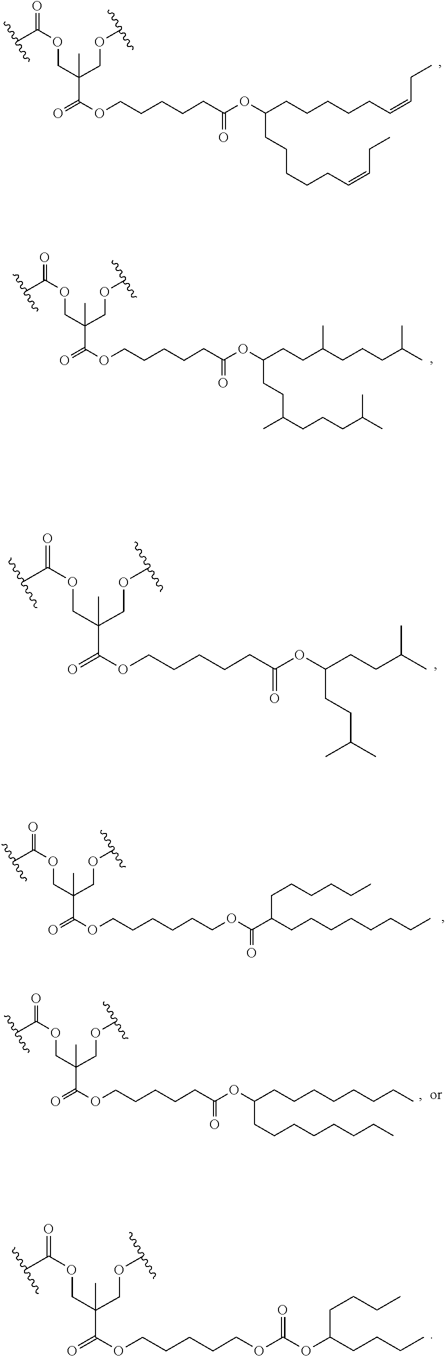 [SMILES: *OCC(C)(COC(*)=O)C(=O)OCCCCCC(=O)OC(CCCCC/C=C\CC)CCCCC/C=C\CC ; *OCC(C)(COC(*)=O)C(=O)OCCCCCC(=O)OC(CCC(C)CCCC(C)C)CCC(C)CCCC(C)C ; *OCC(C)(COC(*)=O)C(=O)OCCCCCC(=O)OC(CCC(C)C)CCC(C)C ; *OCC(C)(COC(*)=O)C(=O)OCCCCCCOC(=O)C(CCCCCC)CCCCCCCC ; *OCC(C)(COC(*)=O)C(=O)OCCCCCC(=O)OC(CCCCCCCC)CCCCCCCC ; *OCC(C)(COC(*)=O)C(=O)OCCCCCOC(=O)OC(CCCC)CCCC]

In embodiments, LP¹ is independently

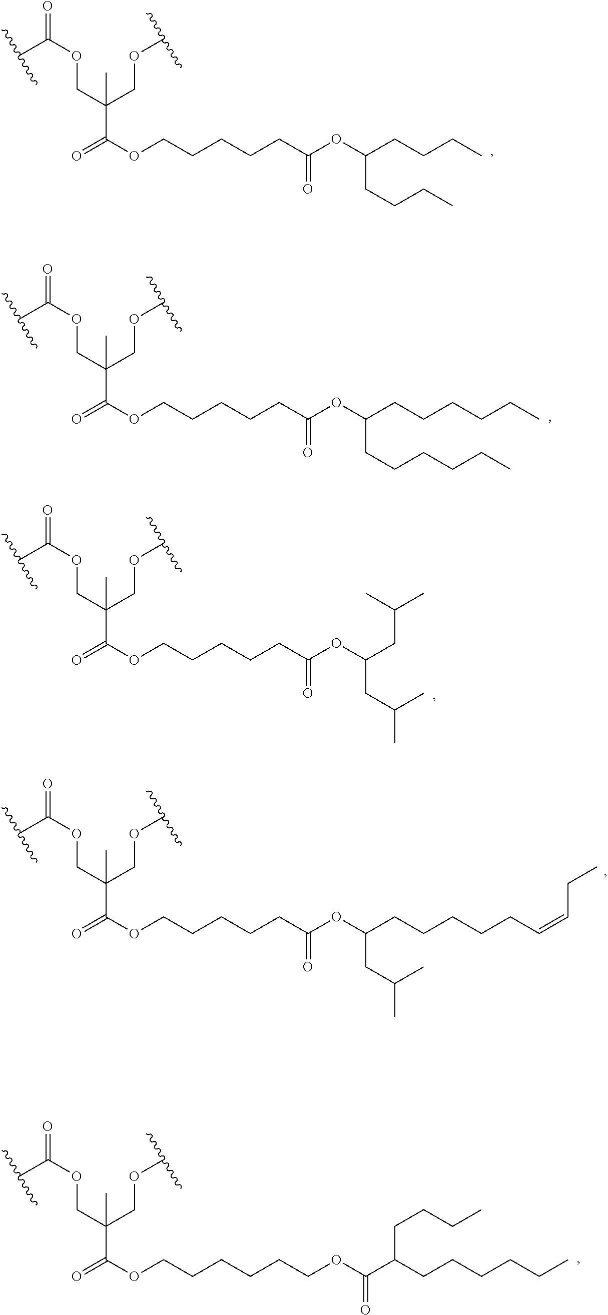 [SMILES: *OCC(C)(COC(*)=O)C(=O)OCCCCCC(=O)OC(CCCC)CCCC ; *OCC(C)(COC(*)=O)C(=O)OCCCCCC(=O)OC(CCCCCC)CCCCCC ; *OCC(C)(COC(*)=O)C(=O)OCCCCCC(=O)OC(CC(C)C)CC(C)C ; *OCC(C)(COC(*)=O)C(=O)OCCCCCC(=O)OC(CCCCC/C=C\CC)CC(C)C ; *OCC(C)(COC(*)=O)C(=O)OCCCCCCOC(=O)C(CCCC)CCCCCC]

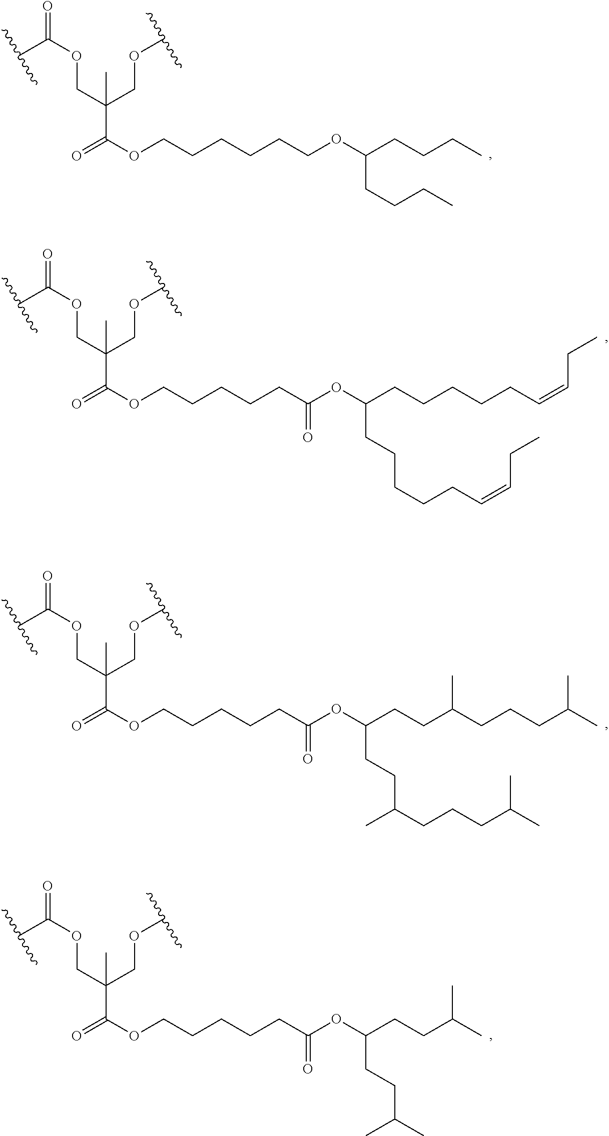 [SMILES: *OCC(C)(COC(*)=O)C(=O)OCCCCCCOC(CCCC)CCCC ; *OCC(C)(COC(*)=O)C(=O)OCCCCCC(=O)OC(CCCCC/C=C\CC)CCCCC/C=C\CC ; *OCC(C)(COC(*)=O)C(=O)OCCCCCC(=O)OC(CCC(C)CCCC(C)C)CCC(C)CCCC(C)C ; *OCC(C)(COC(*)=O)C(=O)OCCCCCC(=O)OC(CCC(C)C)CCC(C)C]

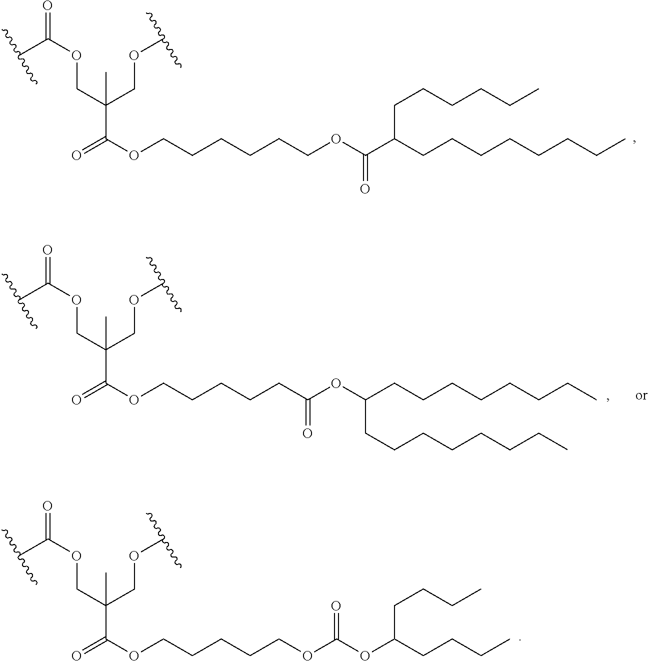 [SMILES: *OCC(C)(COC(*)=O)C(=O)OCCCCCCOC(=O)C(CCCCCC)CCCCCCCC ; *OCC(C)(COC(*)=O)C(=O)OCCCCCC(=O)OC(CCCCCCCC)CCCCCCCC ; *OCC(C)(COC(*)=O)C(=O)OCCCCCOC(=O)OC(CCCC)CCCC]

In embodiments, LP¹ is independently

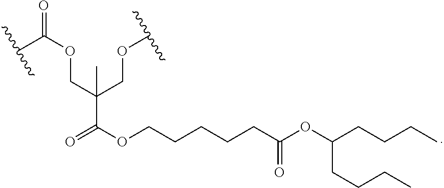 [SMILES: *OCC(C)(COC(*)=O)C(=O)OCCCCCC(=O)OC(CCCC)CCCC]

In embodiments, LP¹ is independently

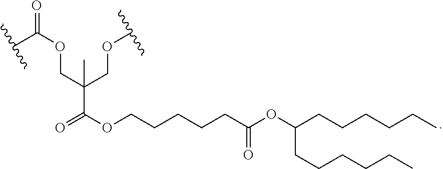 [SMILES: *OCC(C)(COC(*)=O)C(=O)OCCCCCC(=O)OC(CCCCCC)CCCCCC]

In embodiments, LP¹ is independently

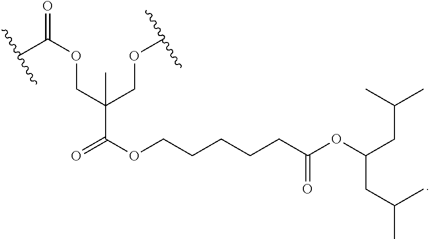 [SMILES: *OCC(C)(COC(*)=O)C(=O)OCCCCCC(=O)OC(CC(C)C)CC(C)C]

In embodiments, LP¹ is independently

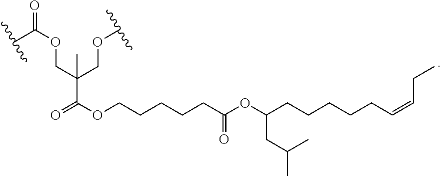 [SMILES: *OCC(C)(COC(*)=O)C(=O)OCCCCCC(=O)OC(CCCCC/C=C\CC)CC(C)C]

In embodiments, LP¹ is independently

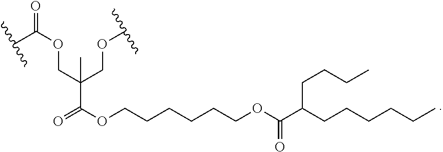 [SMILES: *OCC(C)(COC(*)=O)C(=O)OCCCCCCOC(=O)C(CCCC)CCCCCC]

In embodiments, LP¹ is independently

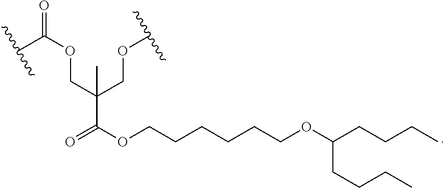 [SMILES: *OCC(C)(COC(*)=O)C(=O)OCCCCCCOC(CCCC)CCCC]

In embodiments, LP¹ is independently

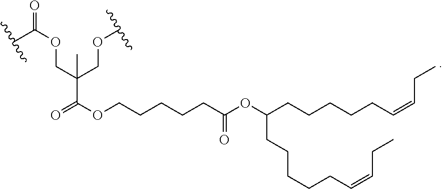 [SMILES: *OCC(C)(COC(*)=O)C(=O)OCCCCCC(=O)OC(CCCCC/C=C\CC)CCCCC/C=C\CC]

In embodiments, LP¹ is independently

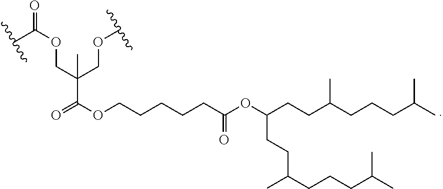 [SMILES: *OCC(C)(COC(*)=O)C(=O)OCCCCCC(=O)OC(CCC(C)CCCC(C)C)CCC(C)CCCC(C)C]

In embodiments, LP¹ is independently

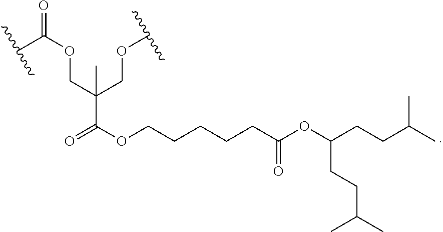 [SMILES: *OCC(C)(COC(*)=O)C(=O)OCCCCCC(=O)OC(CCC(C)C)CCC(C)C]

In embodiments, LP¹ is independently

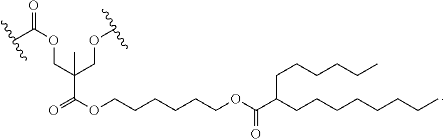 [SMILES: *OCC(C)(COC(*)=O)C(=O)OCCCCCCOC(=O)C(CCCCCC)CCCCCCCC]

In embodiments, LP¹ is independently

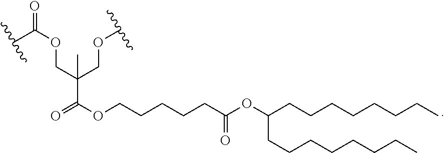 [SMILES: *OCC(C)(COC(*)=O)C(=O)OCCCCCC(=O)OC(CCCCCCCC)CCCCCCCC]

In embodiments, LP¹ is independently

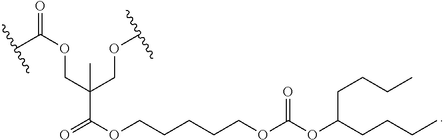 [SMILES: *OCC(C)(COC(*)=O)C(=O)OCCCCCOC(=O)OC(CCCC)CCCC]

In embodiments, LP² is independently

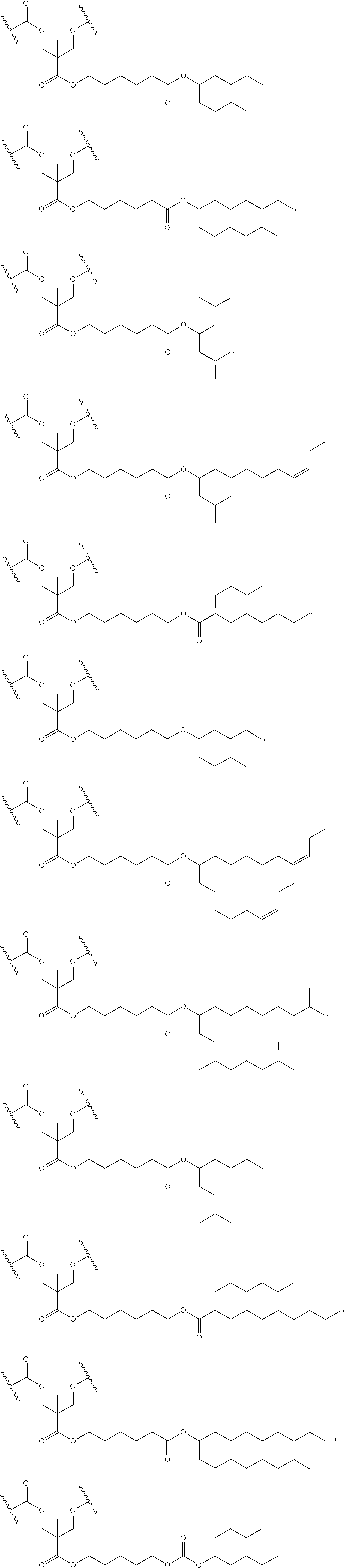

In embodiments, LP² is independently

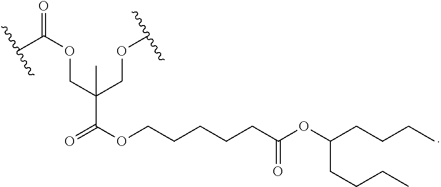 [SMILES: *OCC(C)(COC(*)=O)C(=O)OCCCCCC(=O)OC(CCCC)CCCC]

In embodiments, LP² is independently

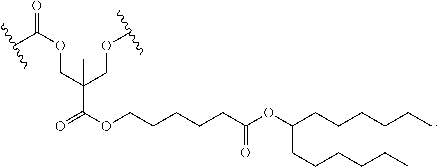 [SMILES: *OCC(C)(COC(*)=O)C(=O)OCCCCCC(=O)OC(CCCCCC)CCCCCC]

In embodiments, LP² is independently

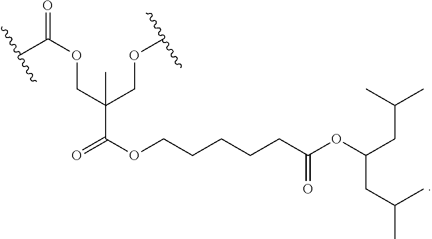 [SMILES: *OCC(C)(COC(*)=O)C(=O)OCCCCCC(=O)OC(CC(C)C)CC(C)C]

In embodiments, LP² is independently

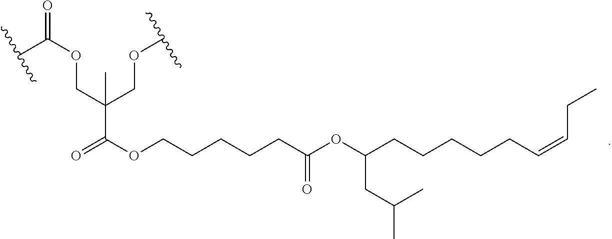 [SMILES: *OCC(C)(COC(*)=O)C(=O)OCCCCCC(=O)OC(CCCCC/C=C\CC)CC(C)C]

In embodiments, LP² is independently

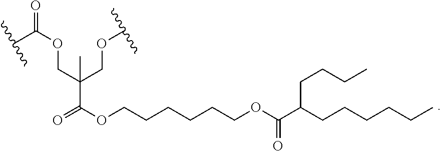 [SMILES: *OCC(C)(COC(*)=O)C(=O)OCCCCCCOC(=O)C(CCCC)CCCCCC]

In embodiments, LP² is independently

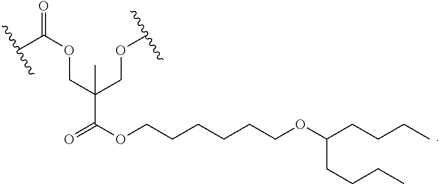 [SMILES: *OCC(C)(COC(*)=O)C(=O)OCCCCCCOC(CCCC)CCCC]

In embodiments, LP² is independently

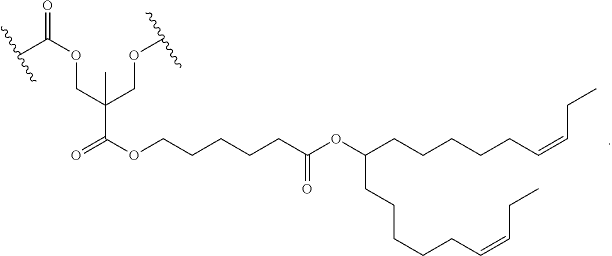 [SMILES: *OCC(C)(COC(*)=O)C(=O)OCCCCCC(=O)OC(CCCCC/C=C\CC)CCCCC/C=C\CC]

In embodiments, LP² is independently

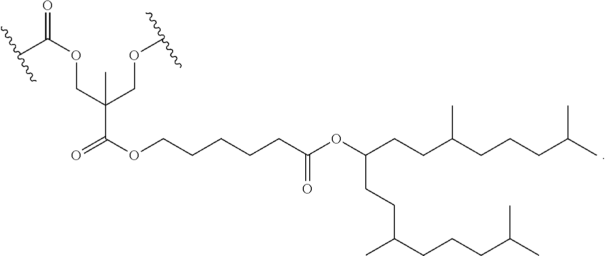 [SMILES: *OCC(C)(COC(*)=O)C(=O)OCCCCCC(=O)OC(CCC(C)CCCC(C)C)CCC(C)CCCC(C)C]

In embodiments, LP² is independently

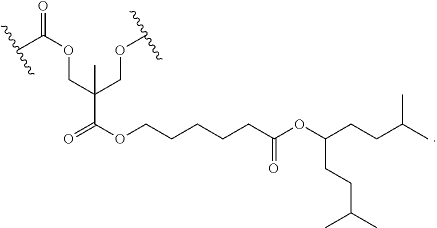 [SMILES: *OCC(C)(COC(*)=O)C(=O)OCCCCCC(=O)OC(CCC(C)C)CCC(C)C]

In embodiments, LP² is independently

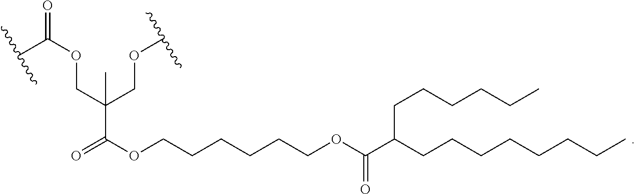 [SMILES: *OCC(C)(COC(*)=O)C(=O)OCCCCCCOC(=O)C(CCCCCC)CCCCCCCC]

In embodiments, LP² is independently

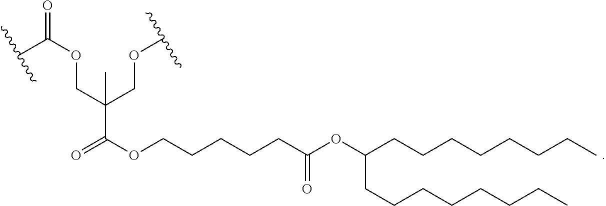 [SMILES: *OCC(C)(COC(*)=O)C(=O)OCCCCCC(=O)OC(CCCCCCCC)CCCCCCCC]

In embodiments, LP² is independently

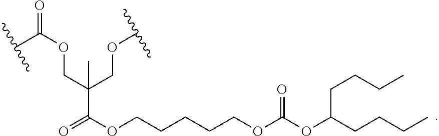 [SMILES: *OCC(C)(COC(*)=O)C(=O)OCCCCCOC(=O)OC(CCCC)CCCC]

In embodiments, each LP¹ or LP² is independently

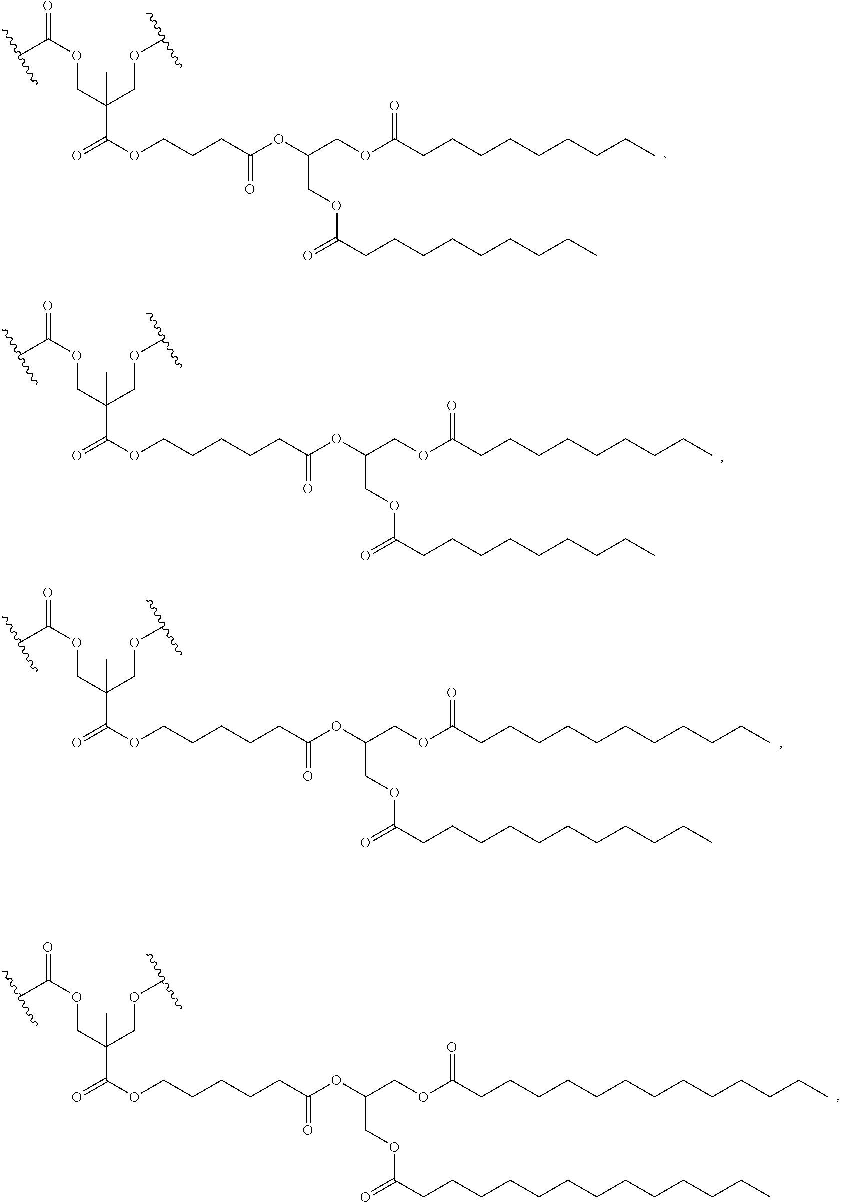 [SMILES: *OCC(C)(COC(*)=O)C(=O)OCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCCCC)COC(=O)CCCCCCCCCCC ; *OCC(C)(COC(*)=O)C(=O)OCCCCCC(=O)OC(COC(=O)CCCCCCCCCCCCC)COC(=O)CCCCCCCCCCCCC]

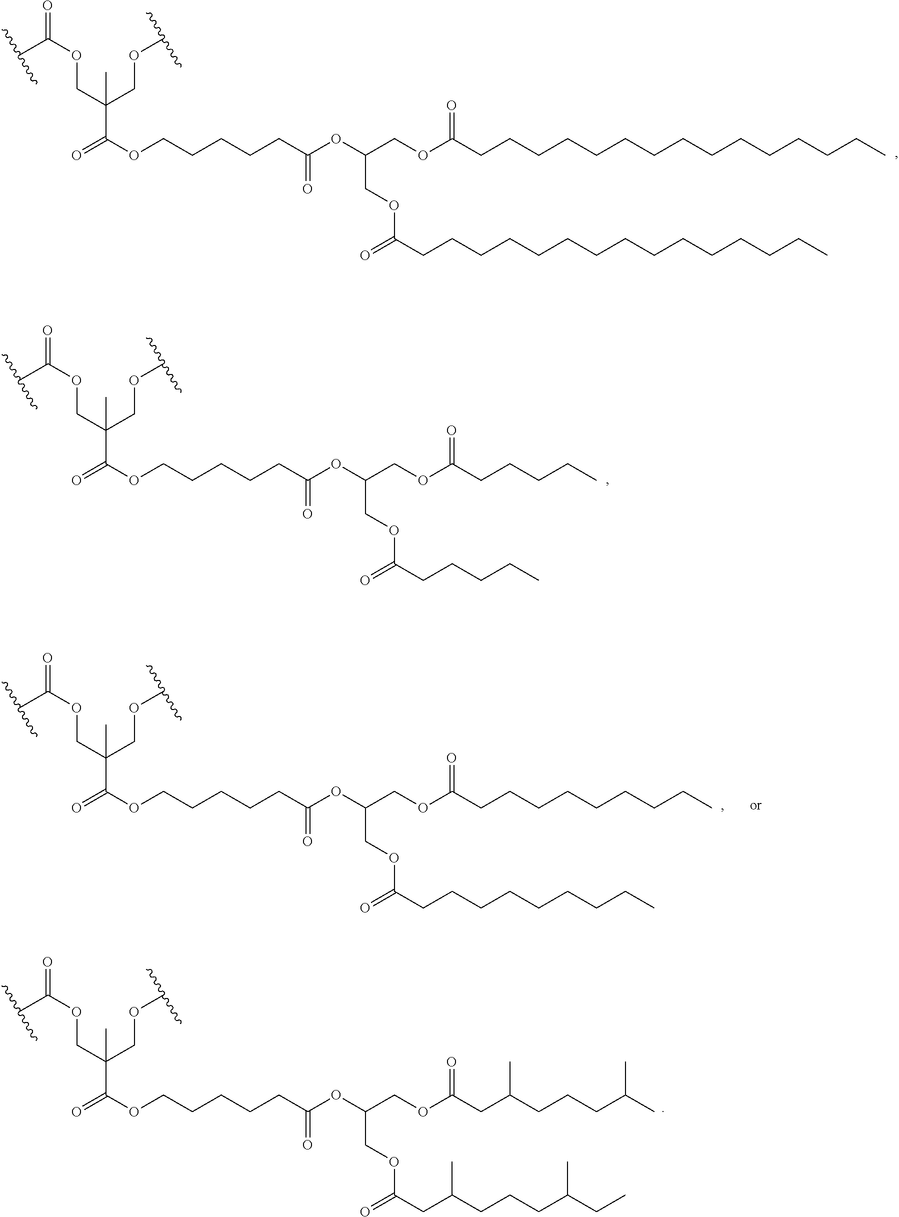 [SMILES: *OCC(C)(COC(*)=O)C(=O)OCCCCCC(=O)OC(COC(=O)CCCCCCCCCCCCCCC)COC(=O)CCCCCCCCCCCCCCC ; *OCC(C)(COC(*)=O)C(=O)OCCCCCC(=O)OC(COC(=O)CCCCC)COC(=O)CCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CC(C)CCCC(C)C)COC(=O)CC(C)CCCC(C)CC]

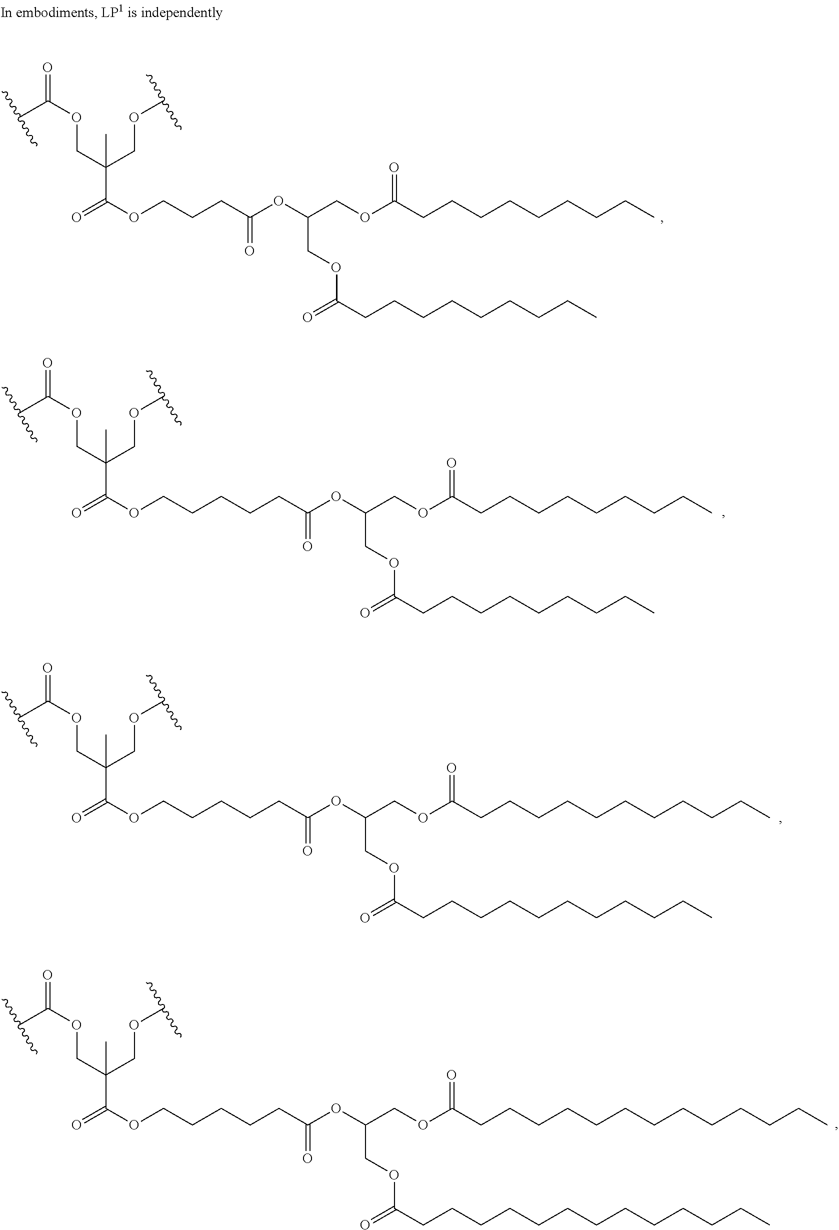 [SMILES: *OCC(C)(COC(*)=O)C(=O)OCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCCCC)COC(=O)CCCCCCCCCCC ; *OCC(C)(COC(*)=O)C(=O)OCCCCCC(=O)OC(COC(=O)CCCCCCCCCCCCC)COC(=O)CCCCCCCCCCCCC]

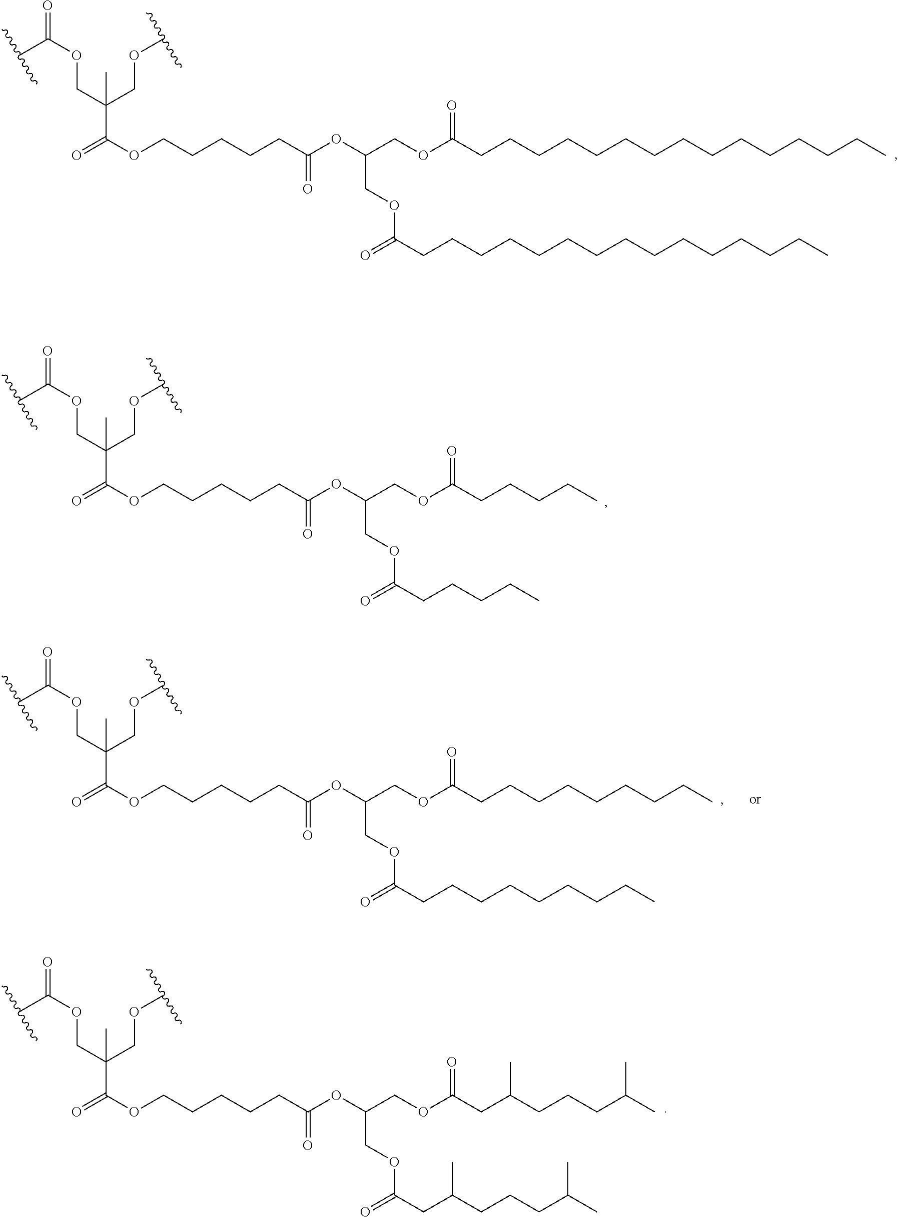 [SMILES: *OCC(C)(COC(*)=O)C(=O)OCCCCCC(=O)OC(COC(=O)CCCCCCCCCCCCCCC)COC(=O)CCCCCCCCCCCCCCC ; *OCC(C)(COC(*)=O)C(=O)OCCCCCC(=O)OC(COC(=O)CCCCC)COC(=O)CCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CC(C)CCCC(C)C)COC(=O)CC(C)CCCC(C)C]

In embodiments, LP¹ is independently

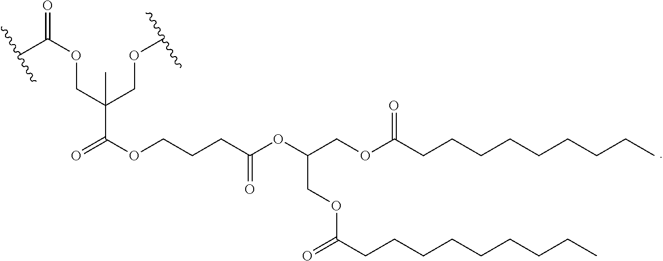 [SMILES: *OCC(C)(COC(*)=O)C(=O)OCCCC(=O)OC(COC(=O)CCCCCCCCC)COC(=O)CCCCCCCCC]

In embodiments, LP¹ is independently

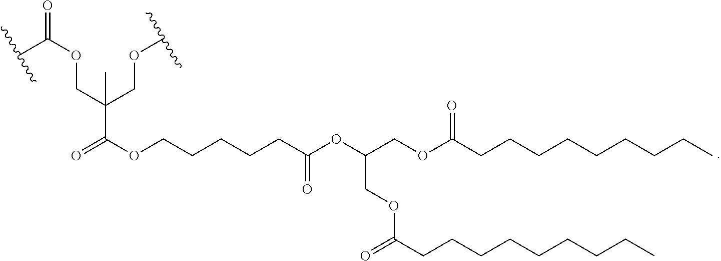 [SMILES: *OCC(C)(COC(*)=O)C(=O)OCCCCCC(=O)OC(COC(=O)CCCCCCCCC)COC(=O)CCCCCCCCC]

In embodiments, LP¹ is independently

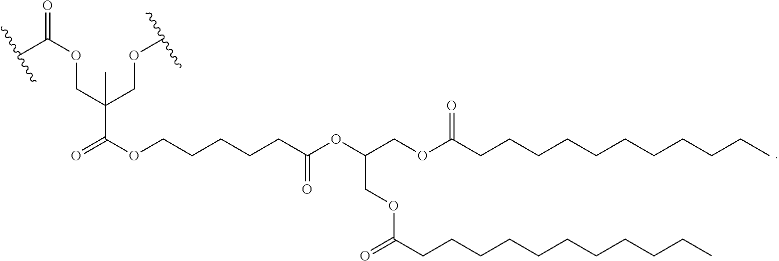 [SMILES: *OCC(C)(COC(*)=O)C(=O)OCCCCCC(=O)OC(COC(=O)CCCCCCCCCCC)COC(=O)CCCCCCCCCCC]

In embodiments, LP¹ is independently

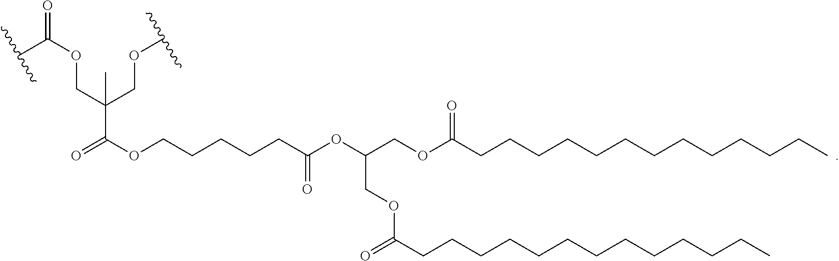 [SMILES: *OCC(C)(COC(*)=O)C(=O)OCCCCCC(=O)OC(COC(=O)CCCCCCCCCCCCC)COC(=O)CCCCCCCCCCCCC]

In embodiments, LP¹ is independently

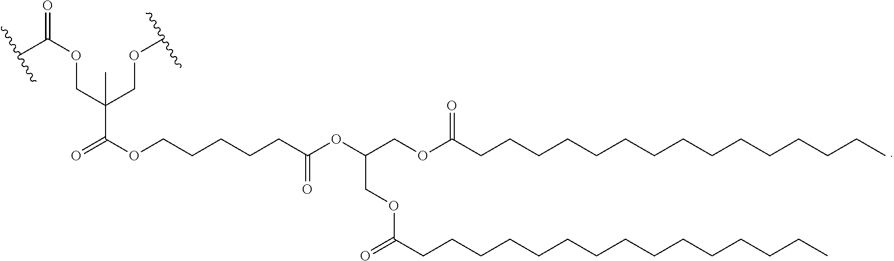 [SMILES: *OCC(C)(COC(*)=O)C(=O)OCCCCCC(=O)OC(COC(=O)CCCCCCCCCCCCCCC)COC(=O)CCCCCCCCCCCCCCC]

In embodiments, LP¹ is independently

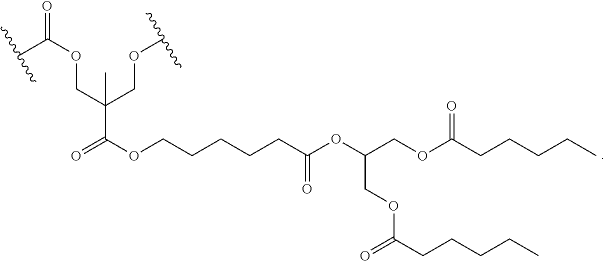 [SMILES: *OCC(C)(COC(*)=O)C(=O)OCCCCCC(=O)OC(COC(=O)CCCCC)COC(=O)CCCCC]

In embodiments, LP¹ is independently

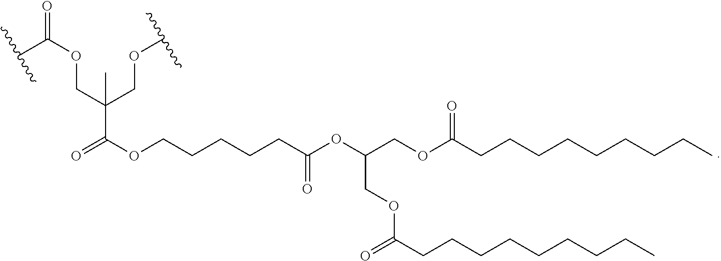 [SMILES: *OCC(C)(COC(*)=O)C(=O)OCCCCCC(=O)OC(COC(=O)CCCCCCCCC)COC(=O)CCCCCCCCC]

In embodiments, LP¹ is independently

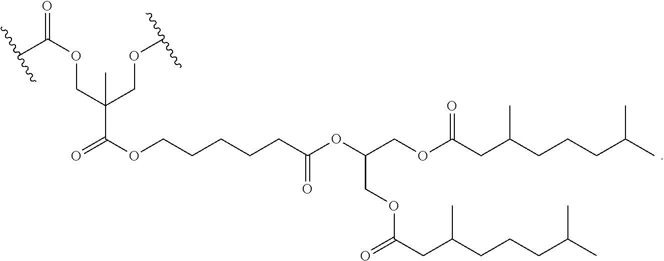 [SMILES: *OCC(C)(COC(*)=O)C(=O)OCCCCCC(=O)OC(COC(=O)CC(C)CCCC(C)C)COC(=O)CC(C)CCCC(C)C]

In embodiments, LP² is independently

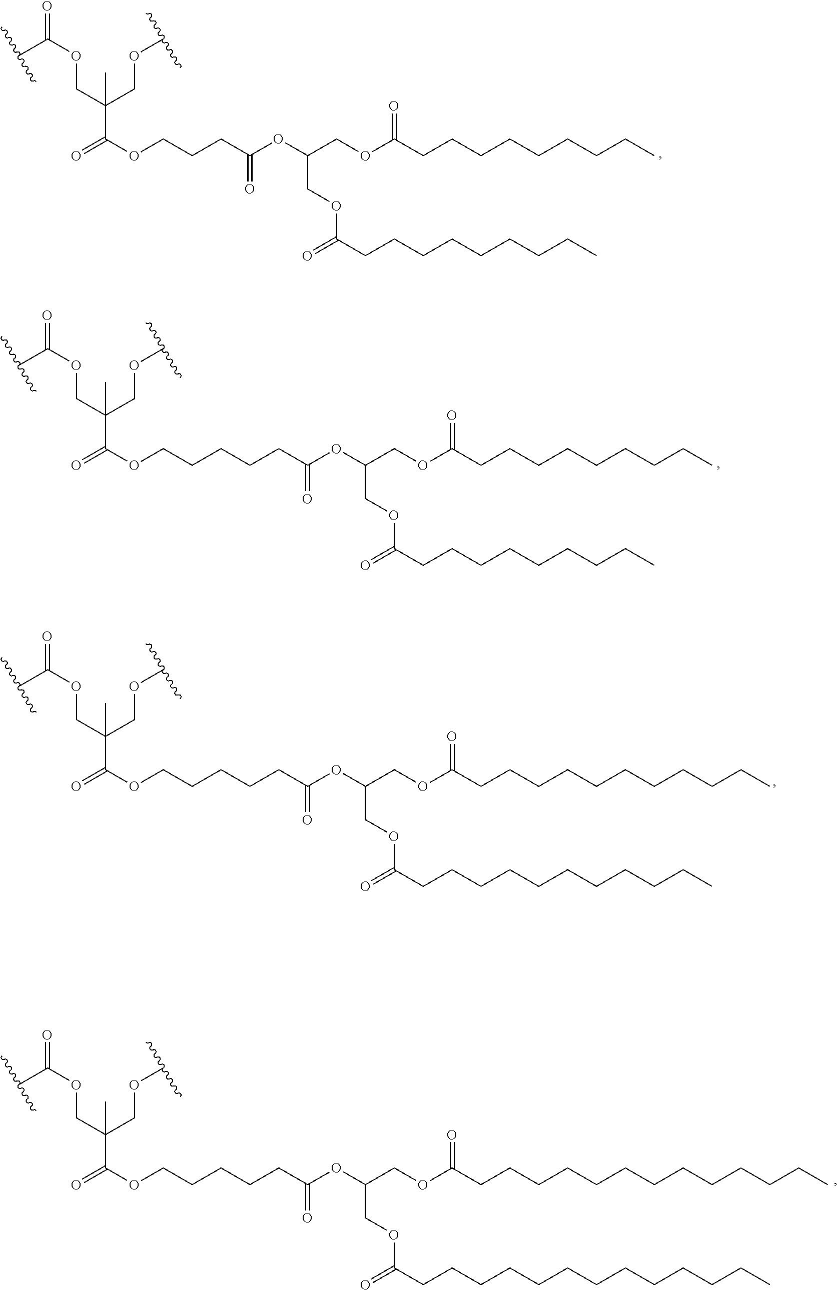 [SMILES: *OCC(C)(COC(*)=O)C(=O)OCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CCCCCCCCCCC)COC(=O)CCCCCCCCCCC ; *OCC(C)(COC(*)=O)C(=O)OCCCCCC(=O)OC(COC(=O)CCCCCCCCCCCCC)COC(=O)CCCCCCCCCCCCC]

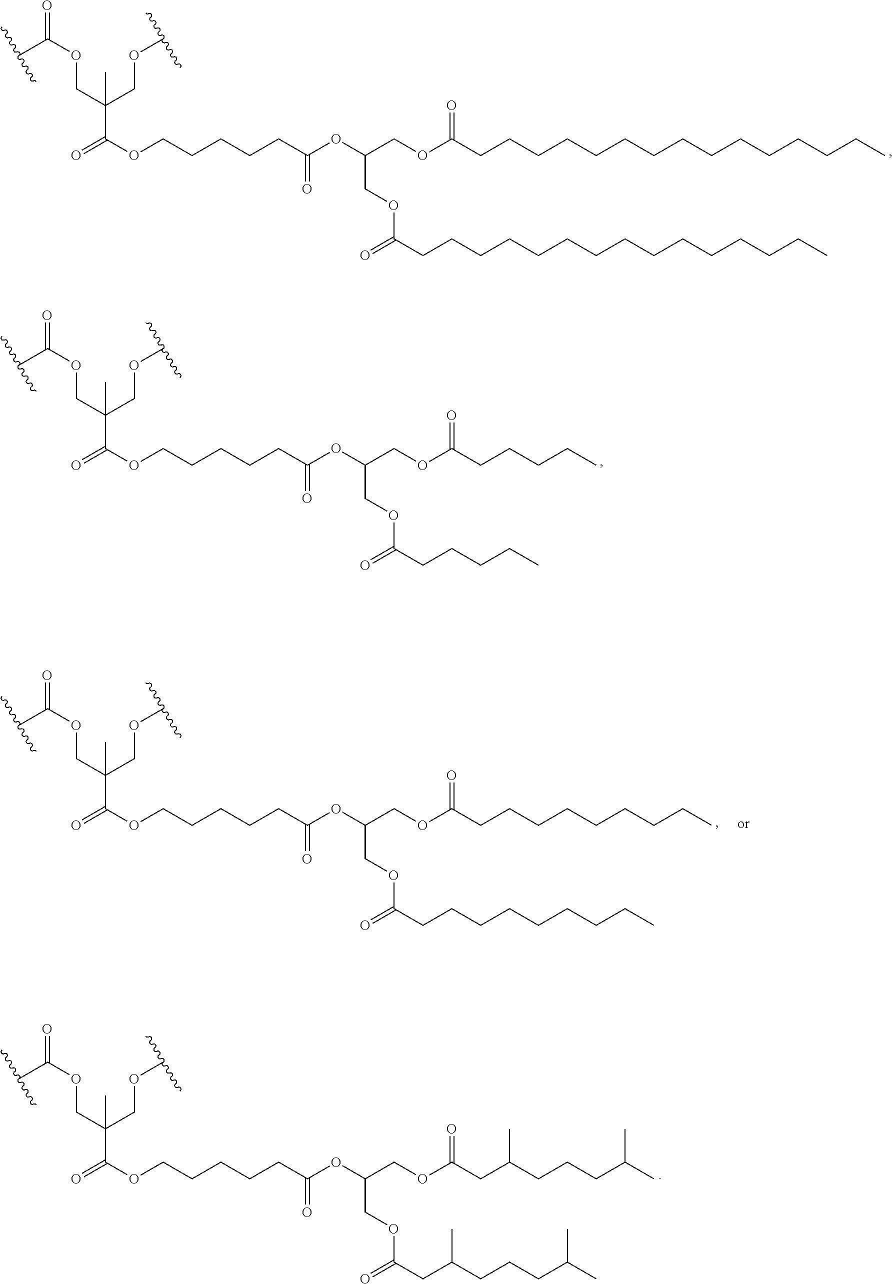 [SMILES: *OCC(C)(COC(*)=O)C(=O)OCCCCCC(=O)OC(COC(=O)CCCCCCCCCCCCCCC)COC(=O)CCCCCCCCCCCCCCC ; *OCC(C)(COC(*)=O)C(=O)OCCCCCC(=O)OC(COC(=O)CCCCC)COC(=O)CCCCC ; *OCC(C)(COC(*)=O)C(=O)OCCCCCC(=O)OC(COC(=O)CCCCCCCCC)COC(=O)CCCCCCCCC ; *OCC(C)(COC(*)=O)C(=O)OCCCCCC(=O)OC(COC(=O)CC(C)CCCC(C)C)COC(=O)CC(C)CCCC(C)C]

In embodiments, LP² is independently a

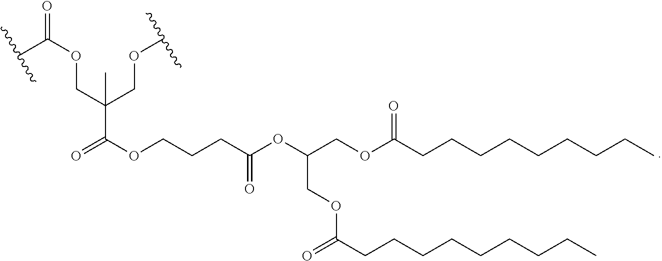 [SMILES: *OCC(C)(COC(*)=O)C(=O)OCCCC(=O)OC(COC(=O)CCCCCCCCC)COC(=O)CCCCCCCCC]

In embodiments, LP² is independently

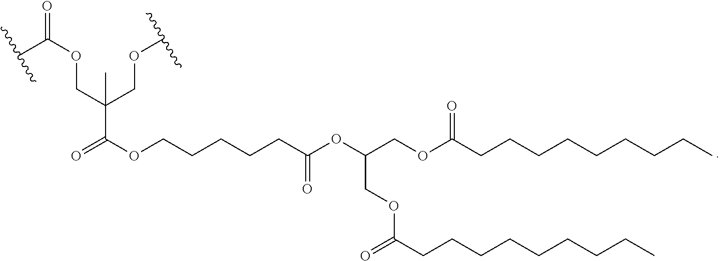 [SMILES: *OCC(C)(COC(*)=O)C(=O)OCCCCCC(=O)OC(COC(=O)CCCCCCCCC)COC(=O)CCCCCCCCC]

In embodiments, LP² is independently

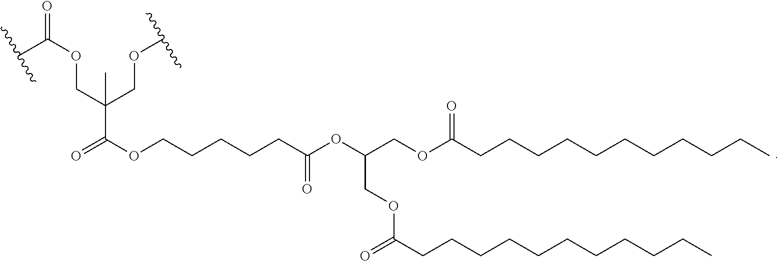 [SMILES: *OCC(C)(COC(*)=O)C(=O)OCCCCCC(=O)OC(COC(=O)CCCCCCCCCCC)COC(=O)CCCCCCCCCCC]

In embodiments, LP² is independently

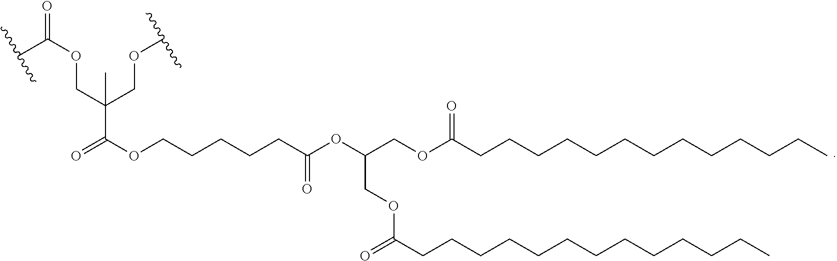 [SMILES: *OCC(C)(COC(*)=O)C(=O)OCCCCCC(=O)OC(COC(=O)CCCCCCCCCCCCC)COC(=O)CCCCCCCCCCCCC]

In embodiments, LP² is independently

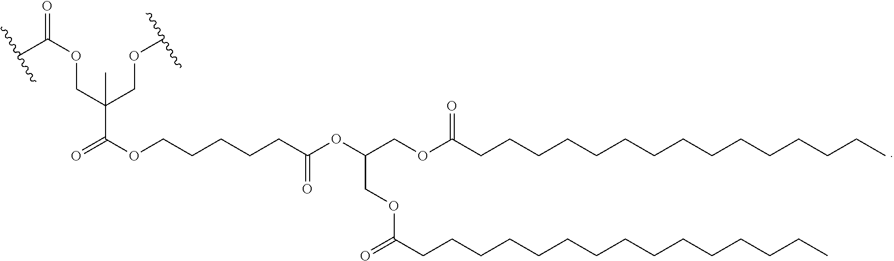 [SMILES: *OCC(C)(COC(*)=O)C(=O)OCCCCCC(=O)OC(COC(=O)CCCCCCCCCCCCCCC)COC(=O)CCCCCCCCCCCCCCC]

In embodiments, LP² is independently

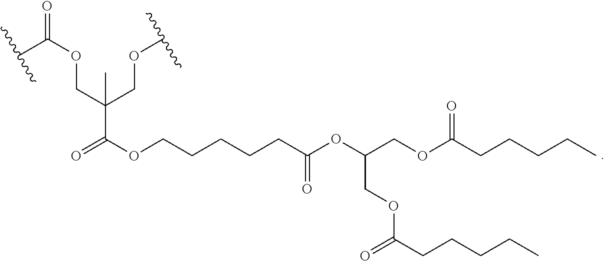 [SMILES: *OCC(C)(COC(*)=O)C(=O)OCCCCCC(=O)OC(COC(=O)CCCCC)COC(=O)CCCCC]

In embodiments, LP² is independently

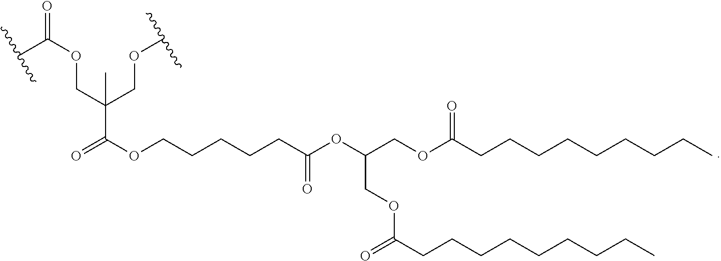 [SMILES: *OCC(C)(COC(*)=O)C(=O)OCCCCCC(=O)OC(COC(=O)CCCCCCCCC)COC(=O)CCCCCCCCC]

In embodiments, LP² is independently

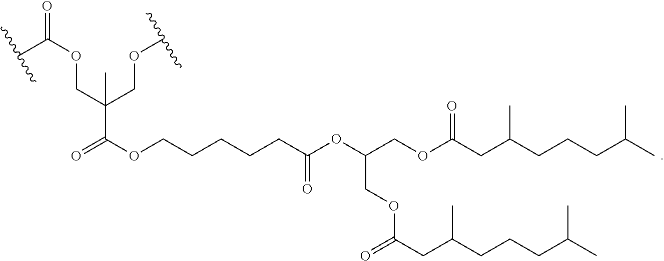 [SMILES: *OCC(C)(COC(*)=O)C(=O)OCCCCCC(=O)OC(COC(=O)CC(C)CCCC(C)C)COC(=O)CC(C)CCCC(C)C]

In embodiments of the co-oligomer complexed with nucleic acid, the poly(alpha-aminoester) monomer includes a first nucleophilic moiety and a first electrophilic moiety, wherein the first nucleophilic moiety is reactive with the first electrophilic moiety within a pH range and is not substantially reactive with the electrophilic moiety outside that pH range (e.g., pH about 1-5, pH about 5-7 or pH about 7-10). In embodiments, the pH range within which the first nucleophilic moiety is most reactive with the first electrophilic moiety is: pH 1-3, pH 2-4, pH 3-5, pH 4-6, pH 5-7, pH 6-8, pH 7-9, or pH 8-10. A nucleophilic moiety is used in accordance with its plain ordinary meaning in chemistry and refers to a moiety (e.g., functional group) capable of donating electrons.

In embodiments, the pH range within which the first nucleophilic moiety is most reactive with the first electrophilic moiety is pH 1-3. In embodiments, the pH range within which the first nucleophilic moiety is most reactive with the first electrophilic moiety is pH 2-4. In embodiments, the pH range within which the first nucleophilic moiety is most reactive with the first electrophilic moiety is pH 3-5. In embodiments, the pH range within which the first nucleophilic moiety is most reactive with the first electrophilic moiety is pH 4-6. In embodiments, the pH range within which the first nucleophilic moiety is most reactive with the first electrophilic moiety is pH 5-7. In embodiments, the pH range within which the first nucleophilic moiety is most reactive with the first electrophilic moiety is pH 6-8. In embodiments, the pH range within which the first nucleophilic moiety is most reactive with the first electrophilic moiety is pH 7-9. In embodiments, the pH range within which the first nucleophilic moiety is most reactive with the first electrophilic moiety is pH 8-10. In embodiments, the pH is 1. In embodiments, the pH is 2. In embodiments, the pH is 3. In embodiments, the pH is 4. In embodiments, the pH is 5. In embodiments, the pH is 6. In embodiments, the pH is 7. In embodiments, the pH is 8. In embodiments, the pH is 9. In embodiments, the pH is 10. In embodiments, the pH is about 1. In embodiments, the pH is about 2. In embodiments, the pH is about 3. In embodiments, the pH is about 4. In embodiments, the pH is about 5. In embodiments, the pH is about 6. In embodiments, the pH is about 7. In embodiments, the pH is about 8. In embodiments, the pH is about 9. In embodiments, the pH is about 10.

In embodiments, the first nucleophilic moiety is substantially protonated at low pH (e.g., pH about 1 to about 5). In embodiments, the first nucleophilic moiety is substantially protonated in the range pH 5-7. In embodiments, the first nucleophilic moiety is cationic. In embodiments, the first nucleophilic moiety includes a cationic nitrogen (e.g. a cationic amine).

In embodiments, the first nucleophilic moiety can be attached to a pH-labile protecting group. The term “pH-labile protecting group” or the like refers, in the usual and customary sense, to a chemical moiety capable of protecting another functionality to which it is attached, and which protecting group can be cleaved or otherwise inactivated as a protecting group under certain pH conditions (e.g., such as decreasing the pH). In embodiments, the pH-labile protecting group is —CO₂-t-Bu, a group removed under acidic conditions (e.g., pH below 7). Additional nucleophile protecting groups could also include those that are cleaved by light, heat, nucleophile, and bases.

In embodiments of the co-oligomer complexed with nucleic acid disclosed above and embodiments thereof, the poly(alpha-aminoester) monomer has the structure of Formula (II) following:

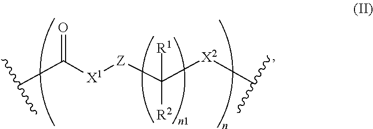

wherein:
n is an integer of 2 or 50;
n1 is an integer from 0 to 50;
Z is —NR³—, —N⁺(R¹³)(H)—, or

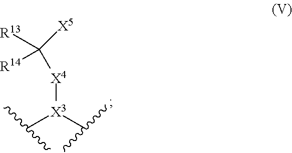

wherein X³ is —N—; X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—; X⁵ is —N⁺H₂(R¹³); and R¹³, R¹⁴, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
X¹ is —C(R⁵)(R⁶)—;
X² is —O— or —S—, and
R¹, R², R⁵, and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n is an integer in the range 2-100, 2-90, 2-80, 2-70, 2-60, 2-50, 2-40, 2-30, 2-2, or 2-10. In embodiments, n1 is an integer in the range 0-25, 0-10, 0-5. In embodiments, n1 is 0, 1, 2, 3, 4 or 5. In embodiments, n1 is 1 or 2.

In embodiments, Z is —NR¹³—, or —N¹(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, Z is —NR¹³—. In embodiments, Z is —N⁺(R¹³)(H)—.

In embodiments, R¹, R², R⁵, and R⁶ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R⁵, and R⁶ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R⁵, and R⁶ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R⁵, and R⁶ are independently hydrogen or unsubstituted alkyl (e.g., C₁-C₆ alkyl). In embodiments, R¹, R², R⁵, and R⁶ are hydrogen.

In embodiments of the co-oligomer complexed with nucleic acid disclosed herein and embodiments thereof, the poly(alpha-aminoester) monomer has the structure of Formula (III) following:

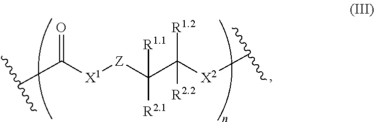

wherein:
n is an integer of 2 or more;
Z is —NR³—, —N⁺(R¹³)(H)—, or

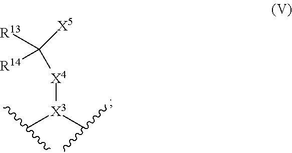

wherein X³ is —N—; X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—; X⁵ is —N⁺H₂(R¹³); R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R¹³, R¹⁴, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
X¹ is —C(R⁵)(R⁶)—;
X² is —O— or —S—, and
R^{1.1}, R^{1.2}, R^{2.1}, R^{2.2}, R⁵, and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In embodiments, R. R¹², R²¹, R²², R⁵, and R⁶ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{1.1}, R^{1.2}, R^{2.1}, R^{2.2}, R⁵, and R⁶ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1.1}, R^{1.2}, R^{2.1}, R^{2.2}, R⁵, and R⁶ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R^{1.1}, R^{1.2}, R^{2.1}, R^{2.2}, R⁵, and R⁶ are independently hydrogen or unsubstituted alkyl (e.g., C₁-C₆ alkyl). In embodiments, R^{1.1}, R^{1.2}, R^{2.1}, R^{2.2}, R⁵, and R⁶ are hydrogen.

In embodiments, Z is —NR¹³—, or —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, Z is —NR¹³—. In embodiments, Z is —N⁺(R¹³)(H)—.

In embodiments, R¹³ is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³ is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹³ is independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³ is independently hydrogen or unsubstituted alkyl (e.g., C₁-C₆ alkyl). In embodiments, R¹³ is hydrogen.

In embodiments of the co-oligomer complexed with nucleic acid disclosed herein and embodiments thereof, the poly(alpha-aminoester) monomer has the structure of Formula (IV) following:

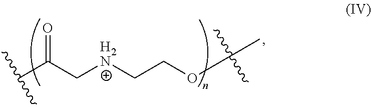

wherein n is an integer of 2 or more.

In embodiments, n is an integer in the range 2-100, 2-90, 2-80, 2-70, 2-60, 2-50, 2-40, 2-30, 2-20, or 2-10. In embodiments, n is an integer in the range 2-100 or 2-50. In some embodiments, n is an integer from 2 to 50. In embodiments, n is an integer from 2 to 15.

In embodiments, Z is

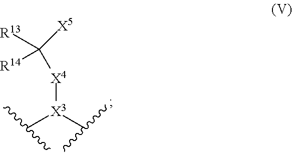

wherein:
X³ is —N—;
X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—; X⁵ is —N⁺H₂(R¹³); and R¹³, R¹⁴, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R¹³, R¹⁴, R¹⁶, R¹⁷, R¹⁸ and R¹⁹ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³, R¹⁴, R¹⁶, R¹⁷, R¹⁸ and R¹⁹ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments R¹³, R¹⁴, R¹⁶, R¹⁷, R¹⁸ and R¹⁹ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³, R¹⁴, R¹⁶, R¹⁷, R¹⁸ and R¹⁹ are independently hydrogen or unsubstituted alkyl (e.g., C₁-C₆ alkyl).

In embodiments, X⁵ is —N⁺H₂(R¹³), wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the poly(alpha-aminoester) monomer has the structure following:

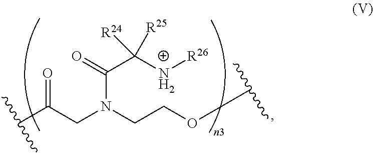

wherein R²⁴, R²⁵ and R²⁶ are independently hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and n3 is an integer from 0 to 50.

In embodiments, R²⁴, R²⁵ and R²⁶ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁴, R²⁵ and R²⁶ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁴, R²⁵ and R²⁶ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R²⁴, R²⁵ and R²⁶ are independently hydrogen or unsubstituted alkyl (e.g., C₁-C₆ alkyl). In embodiments, R²⁴, R²⁵ and R²⁶ are independently hydrogen.

In embodiments, the poly(alpha-aminoester) monomer has a structure

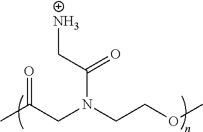

where n is as described herein.

In embodiments, the poly(alpha-aminoester) monomer has the structure following:

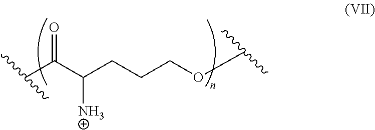

wherein n is an integer of 2 or more.

In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In some embodiments, the poly(alpha-aminoester) monomers can have one of the following formula:

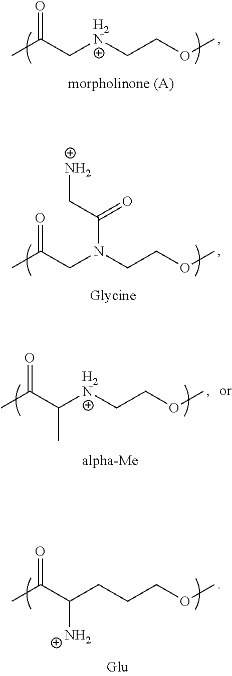

In embodiments, R^{1A} is a substituted or unsubstituted cycloalkyl, substituted or unsubstituted heterocycloalkyl, substituted or unsubstituted aryl, or substituted or unsubstituted heteroaryl. In embodiments, R^{1A} is a substituted or unsubstituted cycloalkyl. In embodiments, R^{1A} is a substituted or unsubstituted heterocycloalkyl. In embodiments, R^{1A} is a substituted or unsubstituted aryl. In embodiments, R^{1A} is a substituted or unsubstituted heteroaryl. In embodiments, R^{1A} is a substituted or unsubstituted phenyl.

In embodiments, R^{A} is represented as Ring A. In some embodiments, the co-oligomer complexed with nucleic acid can have a co-oligomer having the following formula (VIII):

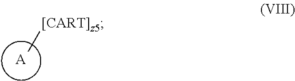

wherein
Ring A is a substituted or unsubstituted cycloalkyl, substituted or unsubstituted heterocycloalkyl, substituted or unsubstituted aryl, or substituted or unsubstituted heteroaryl;
CART has the formula: -L¹-[(LP¹)_{z1}-(AM)_{z2}-(LP²)_{z3}]_{z4}-L²-R^{2A}
wherein,
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a bond or a non-linear branched lipophilic monomer, wherein at least one of LP¹ or LP² is a non-linear branched lipophilic monomer; AM is a poly(alpha-aminoester) monomer; z5 is an integer from 1 to 10; z1 and z3 are independently integers from 0 to 100, wherein at least one of z1 or z3 is not 0; z4 is an integer from 1 to 100, and z2 is an integer from 2 to 100.

In some embodiments, in the above-formula (VIII), Ring A is a substituted or unsubstituted aryl. In some other embodiments, Ring A is a substituted or unsubstituted phenyl. In still some other embodiments, Ring A is a substituted or unsubstituted aryl. In still some other embodiments, Ring A is a substituted or unsubstituted phenyl or naphthalenyl.

In embodiments, Ring A is an unsubstituted aryl (i.e. unsubstituted beyond the CART moiety). In embodiments, Ring A is an unsubstituted phenyl (i.e. unsubstituted beyond the CART moiety). In embodiments, Ring A is an unsubstituted phenyl or naphthalenyl (i.e. unsubstituted beyond the CART moiety). In embodiments, Ring A is a substituted aryl (i.e. substituted in addition to the CART moiety). In embodiments, Ring A is a substituted phenyl (i.e. substituted in addition to the CART moiety). In embodiments, Ring A is a substituted phenyl or naphthalenyl (i.e. substituted in addition to the CART moiety).

In embodiments, the co-oligomer complexed with nucleic acid includes a detectable agent (e.g., fluorophore).

In embodiments, R^{1A} is an aryl substituted with a methoxy linker. In embodiments, R^{1A} is an aryl substituted with a linker (e.g., —CH₂—O—). A non-limiting example wherein R^{1A} is an aryl substituted with a methoxy linker has the formula:

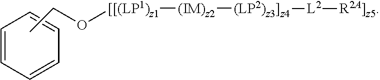

In some embodiments, a co-oligomer has the formula (IX):

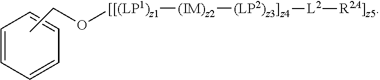

In some embodiments, a co-oligomer has the formula (X):

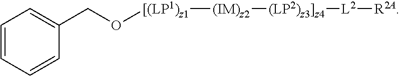

In some embodiments, a co-oligomer can have the formula (XI):

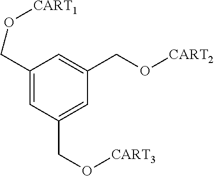

wherein CART₁, CART₂ and CART₃ are independently a CART moiety as defined in formula (VIII) (e.g., -L¹-[(LP¹)_{z1}-(AM)_{z2}-(LP²)_{z3}]_{z4}-L²-R^{2A}). In embodiments each CART moiety is optionally different.

In embodiments, the co-oligomer has the formula:

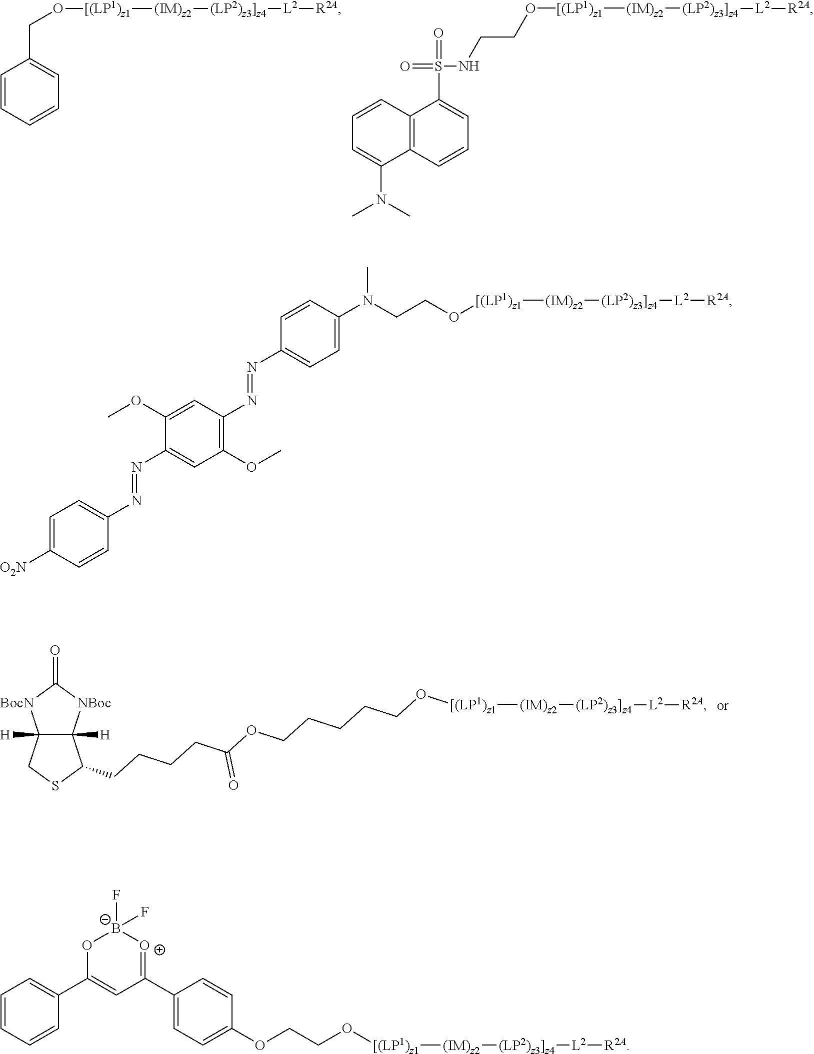

In embodiments, the co-oligomer has the formula:

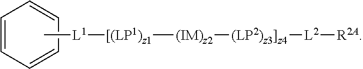

In embodiments, the co-oligomer has the formula:

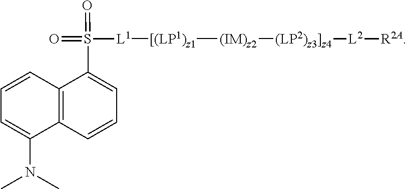

In embodiments, the co-oligomer has the formula:

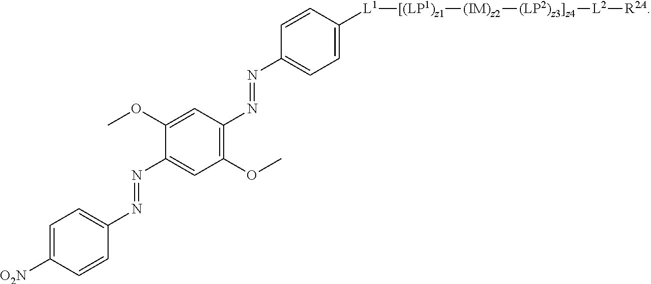

In embodiments, the co-oligomer has the formula:

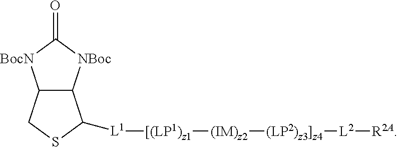

In embodiments, the co-oligomer has the formula:

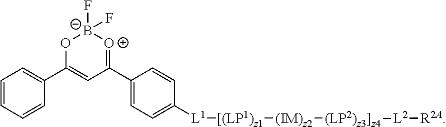

In embodiments, Ring A is substituted with a detectable agent through a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene).

In some embodiments, a co-oligomer can have any of the foregoing formula in which z1 and z3 are independently integers from 0 to 100, wherein at least one of z or z3 is not 0 and z4 is an integer from 1 to 100. In some embodiments, z1, z3 and z4 can be independently integers in the range 2-100, 2-90, 2-80, 2-70, 2-60, 2-50, 2-40, 2-30, 2-2, or 2-10, wherein at least one of z1 or z3 is not 0. In embodiments, z1, z3 and z4 can be independently integers in the range 2-100 or 2-50, wherein at least one of z1 or z3 is not 0.

In embodiments, z1 is an integer from 5 to 30. In embodiments, z1 is 5. In embodiments, z1 is 6. In embodiments, z1 is 7. In embodiments, z1 is 8. In embodiments, z1 is 9. In embodiments, z1 is 10. In embodiments, z1 is 11. In embodiments, z1 is 12. In embodiments, z1 is 13. In embodiments, z1 is 14. In embodiments, z1 is 15. In embodiments, z1 is 16. In embodiments, z1 is 17. In embodiments, z1 is 18. In embodiments, z1 is 19. In embodiments, z1 is 20. In embodiments, z1 is 21. In embodiments, z1 is 22. In embodiments, z1 is 23. In embodiments, z1 is 24. In embodiments, z1 is 25. In embodiments, z1 is 26. In embodiments, z1 is 27. In embodiments, z1 is 28. In embodiments, z1 is 29. In embodiments, z1 is 30.

In embodiments, z3 is an integer from 5 to 40. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11. In embodiments, z3 is 12. In embodiments, z3 is 13. In embodiments, z3 is 14. In embodiments, z3 is 15. In embodiments, z3 is 16. In embodiments, z3 is 17. In embodiments, z3 is 18. In embodiments, z3 is 19. In embodiments, z3 is 20. In embodiments, z3 is 21. In embodiments, z3 is 22. In embodiments, z3 is 23. In embodiments, z3 is 24. In embodiments, z3 is 25. In embodiments, z3 is 26. In embodiments, z3 is 27. In embodiments, z3 is 28. In embodiments, z3 is 29. In embodiments, z3 is 30.

In embodiments, z4 is an integer from 1 to 100. In embodiments, z4 is an integer from 1 to 90. In embodiments, z4 is an integer from 1 to 80. In embodiments, z4 is an integer from 1 to 70. In embodiments, z4 is an integer from 1 to 60. In embodiments, z4 is an integer from 1 to 50. In embodiments, z4 is an integer from 1 to 40. In embodiments, z4 is an integer from 1 to 30. In embodiments, z4 is an integer from 1 to 20. In embodiments, z4 is an integer from or 1 to 10. In embodiments, z4 is an integer from 5 to 100. In embodiments, z4 is an integer from 15 to 100. In embodiments, z4 is an integer from 25 to 100. In embodiments, z4 is an integer from 35 to 100. In embodiments, z4 is an integer from 45 to 100. In embodiments, z4 is an integer from 55 to 100. In embodiments, z4 is an integer from 65 to 100. In embodiments, z4 is an integer from 75 to 100. In embodiments, z4 is an integer from 85 to 100. In embodiments, z4 is an integer from 95 to 100. In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11. In embodiments, z4 is 12. In embodiments, z4 is 13. In embodiments, z4 is 14. In embodiments, z4 is 15. In embodiments, z4 is 16. In embodiments, z4 is 17. In embodiments, z4 is 18. In embodiments, z4 is 19. In embodiments, z4 is 20. In embodiments, z4 is 21. In embodiments, z4 is 22. In embodiments, z4 is 23. In embodiments, z4 is 24. In embodiments, z4 is 25. In embodiments, z4 is 26. In embodiments, z4 is 27. In embodiments, z4 is 28. In embodiments, z4 is 29. In embodiments, z4 is 30. In embodiments, z4 is 31. In embodiments, z4 is 32. In embodiments, z4 is 33. In embodiments, z4 is 34. In embodiments, z4 is 35. In embodiments, z4 is 36. In embodiments, z4 is 37. In embodiments, z4 is 38. In embodiments, z4 is 39. In embodiments, z4 is 40. In embodiments, z4 is 41. In embodiments, z4 is 42. In embodiments, z4 is 43. In embodiments, z4 is 44. In embodiments, z4 is 45. In embodiments, z4 is 46. In embodiments, z4 is 47. In embodiments, z4 is 48. In embodiments, z4 is 49. In embodiments, z4 is 50. In embodiments, z4 is 51. In embodiments, z4 is 52. In embodiments, z4 is 53. In embodiments, z4 is 54. In embodiments, z4 is 55. In embodiments, z4 is 56. In embodiments, z4 is 57. In embodiments, z4 is 58. In embodiments, z4 is 59. In embodiments, z4 is 60. In embodiments, z4 is 61. In embodiments, z4 is 62. In embodiments, z4 is 63. In embodiments, z4 is 64. In embodiments, z4 is 65. In embodiments, z4 is 66. In embodiments, z4 is 67. In embodiments, z4 is 68. In embodiments, z4 is 69. In embodiments, z4 is 70. In embodiments, z4 is 71. In embodiments, z4 is 72. In embodiments, z4 is 73. In embodiments, z4 is 74. In embodiments, z4 is 75. In embodiments, z4 is 76. In embodiments, z4 is 77. In embodiments, z4 is 78. In embodiments, z4 is 79. In embodiments, z4 is 80. In embodiments, z4 is 81. In embodiments, z4 is 82. In embodiments, z4 is 83. In embodiments, z4 is 84. In embodiments, z4 is 85. In embodiments, z4 is 86. In embodiments, z4 is 87. In embodiments, z4 is 88. In embodiments, z4 is 89. In embodiments, z4 is 90. In embodiments, z4 is 91. In embodiments, z4 is 92. In embodiments, z4 is 93. In embodiments, z4 is 94. In embodiments, z4 is 95. In embodiments, z4 is 96. In embodiments, z4 is 97. In embodiments, z4 is 98. In embodiments, z4 is 99. In embodiments, z4 is 100.

In embodiments, n is an integer from 2 to 100. In embodiments, n is an integer from 2 to 90. In embodiments, n is an integer from 2 to 80. In embodiments, n is an integer from 2 to 70. In embodiments, n is an integer from 2 to 60. In embodiments, n is an integer from 2 to 50. In embodiments, n is an integer from 2 to 40. In embodiments, n is an integer from 2 to 30. In embodiments, n is an integer from 2 to 20. In embodiments, n is an integer from or 2 to 10. In embodiments, n is an integer from 5 to 100. In embodiments, n is an integer from 15 to 100. In embodiments, n is an integer from 25 to 100. In embodiments, n is an integer from 35 to 100. In embodiments, n is an integer from 45 to 100. In embodiments, n is an integer from 55 to 100. In embodiments, n is an integer from 65 to 100. In embodiments, n is an integer from 75 to 100. In embodiments, n is an integer from 85 to 100. In embodiments, n is an integer from 95 to 100. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24. In embodiments, n is 25. In embodiments, n is 26. In embodiments, n is 27. In embodiments, n is 28. In embodiments, n is 29. In embodiments, n is 30. In embodiments, n is 31. In embodiments, n is 32. In embodiments, n is 33. In embodiments, n is 34. In embodiments, n is 35. In embodiments, n is 36. In embodiments, n is 37. In embodiments, n is 38. In embodiments, n is 39. In embodiments, n is 40. In embodiments, n is 41. In embodiments, n is 42. In embodiments, n is 43. In embodiments, n is 44. In embodiments, n is 45. In embodiments, n is 46. In embodiments, n is 47. In embodiments, n is 48. In embodiments, n is 49. In embodiments, n is 50. In embodiments, n is 51. In embodiments, n is 52. In embodiments, n is 53. In embodiments, n is 54. In embodiments, n is 55. In embodiments, n is 56. In embodiments, n is 57. In embodiments, n is 58. In embodiments, n is 59. In embodiments, n is 60. In embodiments, n is 61. In embodiments, n is 62. In embodiments, n is 63. In embodiments, n is 64. In embodiments, n is 65. In embodiments, n is 66. In embodiments, n is 67. In embodiments, n is 68. In embodiments, n is 69. In embodiments, n is 70. In embodiments, n is 71. In embodiments, n is 72. In embodiments, n is 73. In embodiments, n is 74. In embodiments, n is 75. In embodiments, n is 76. In embodiments, n is 77. In embodiments, n is 78. In embodiments, n is 79. In embodiments, n is 80. In embodiments, n is 81. In embodiments, n is 82. In embodiments, n is 83. In embodiments, n is 84. In embodiments, n is 85. In embodiments, n is 86. In embodiments, n is 87. In embodiments, n is 88. In embodiments, n is 89. In embodiments, n is 90. In embodiments, n is 91. In embodiments, n is 92. In embodiments, n is 93. In embodiments, n is 94. In embodiments, n is 95. In embodiments, n is 96. In embodiments, n is 97. In embodiments, n is 98. In embodiments, n is 99. In embodiments, n is 100.

In embodiments, n1 is an integer from 0 to 50. In embodiments, n1 is an integer from 2 to 45. In embodiments, n1 is an integer from 0 to 40. In embodiments, n1 is an integer from 0 to 30. In embodiments, n1 is an integer from 0 to 20. In embodiments, n1 is an integer from or 0 to 10. In embodiments, n1 is an integer from 5 to 50. In embodiments, n1 is an integer from 15 to 50. In embodiments, n1 is an integer from 25 to 50. In embodiments, n1 is an integer from 35 to 50. In embodiments, n1 is an integer from 45 to 50. In embodiments, n1 is 0. In embodiments, n1 is 1. In embodiments, n1 is 2. In embodiments, n1 is 3. In embodiments, n1 is 4. In embodiments, n1 is 5. In embodiments, n1 is 6. In embodiments, n1 is 7. In embodiments, n1 is 8. In embodiments, n1 is 9. In embodiments, n1 is 10. In embodiments, n1 is 11. In embodiments, n1 is 12. In embodiments, n1 is 13. In embodiments, n1 is 14. In embodiments, n1 is 15. In embodiments, n1 is 16. In embodiments, n1 is 17. In embodiments, n1 is 18. In embodiments, n1 is 19. In embodiments, n1 is 20. In embodiments, n1 is 21. In embodiments, n1 is 22. In embodiments, n1 is 23. In embodiments, n1 is 24. In embodiments, n1 is 25. In embodiments, n1 is 26. In embodiments, n1 is 27. In embodiments, n1 is 28. In embodiments, n1 is 29. In embodiments, n1 is 30. In embodiments, n1 is 31. In embodiments, n1 is 32. In embodiments, n1 is 33. In embodiments, n1 is 34. In embodiments, n1 is 35. In embodiments, n1 is 36. In embodiments, n1 is 37. In embodiments, n1 is 38. In embodiments, n1 is 39. In embodiments, n1 is 40. In embodiments, n1 is 41. In embodiments, n1 is 42. In embodiments, n1 is 43. In embodiments, n1 is 44. In embodiments, n1 is 45. In embodiments, n1 is 46. In embodiments, n1 is 47. In embodiments, n1 is 48. In embodiments, n1 is 49. In embodiments, n1 is 50.

In embodiments, n is an integer from 1 to 100. In embodiments, n is an integer from 1 to 90. In embodiments, n is an integer from 1 to 80. In embodiments, n is an integer from 1 to 70. In embodiments, n is an integer from 1 to 60. In embodiments, n is an integer from 1 to 50. In embodiments, n is an integer from 1 to 40. In embodiments, n is an integer from 1 to 30. In embodiments, n is an integer from 1 to 20. In embodiments, n is an integer from or 1 to 10. In embodiments, n is an integer from 5 to 100. In embodiments, n is an integer from 15 to 100. In embodiments, n is an integer from 25 to 100. In embodiments, n is an integer from 35 to 100. In embodiments, n is an integer from 45 to 100. In embodiments, n is an integer from 55 to 100. In embodiments, n is an integer from 65 to 100. In embodiments, n is an integer from 75 to 100. In embodiments, n is an integer from 85 to 100. In embodiments, n is an integer from 95 to 100. In embodiments, n2 is 1. In embodiments, n2 is 2. In embodiments, n2 is 3. In embodiments, n2 is 4. In embodiments, n2 is 5. In embodiments, n2 is 6. In embodiments, n2 is 7. In embodiments, n2 is 8. In embodiments, n2 is 9. In embodiments, n2 is 10. In embodiments, n2 is 11. In embodiments, n2 is 12. In embodiments, n2 is 13. In embodiments, n2 is 14. In embodiments, n2 is 15. In embodiments, n2 is 16. In embodiments, n2 is 17. In embodiments, n2 is 18. In embodiments, n2 is 19. In embodiments, n2 is 20. In embodiments, n2 is 21. In embodiments, n2 is 22. In embodiments, n2 is 23. In embodiments, n2 is 24. In embodiments, n2 is 25. In embodiments, n2 is 26. In embodiments, n2 is 27. In embodiments, n2 is 28. In embodiments, n2 is 29. In embodiments, n2 is 30. In embodiments, n2 is 31. In embodiments, n2 is 32. In embodiments, n2 is 33. In embodiments, n2 is 34. In embodiments, n2 is 35. In embodiments, n2 is 36. In embodiments, n2 is 37. In embodiments, n2 is 38. In embodiments, n2 is 39. In embodiments, n2 is 40. In embodiments, n2 is 41. In embodiments, n2 is 42. In embodiments, n2 is 43. In embodiments, n2 is 44. In embodiments, n2 is 45. In embodiments, n2 is 46. In embodiments, n2 is 47. In embodiments, n2 is 48. In embodiments, n2 is 49. In embodiments, n2 is 50. In embodiments, n2 is 51. In embodiments, n2 is 52. In embodiments, n2 is 53. In embodiments, n2 is 54. In embodiments, n2 is 55. In embodiments, n2 is 56. In embodiments, n2 is 57. In embodiments, n2 is 58. In embodiments, n2 is 59. In embodiments, n2 is 60. In embodiments, n2 is 61. In embodiments, n2 is 62. In embodiments, n2 is 63. In embodiments, n2 is 64. In embodiments, n2 is 65. In embodiments, n2 is 66. In embodiments, n2 is 67. In embodiments, n2 is 68. In embodiments, n2 is 69. In embodiments, n2 is 70. In embodiments, n2 is 71. In embodiments, n2 is 72. In embodiments, n2 is 73. In embodiments, n2 is 74. In embodiments, n2 is 75. In embodiments, n2 is 76. In embodiments, n2 is 77. In embodiments, n2 is 78. In embodiments, n2 is 79. In embodiments, n2 is 80. In embodiments, n2 is 81. In embodiments, n2 is 82. In embodiments, n2 is 83. In embodiments, n2 is 84. In embodiments, n2 is 85. In embodiments, n2 is 86. In embodiments, n2 is 87. In embodiments, n2 is 88. In embodiments, n2 is 89. In embodiments, n2 is 90. In embodiments, n2 is 91. In embodiments, n2 is 92. In embodiments, n2 is 93. In embodiments, n2 is 94. In embodiments, n2 is 95. In embodiments, n2 is 96. In embodiments, n2 is 97. In embodiments, n2 is 98. In embodiments, n2 is 99. In embodiments, n2 is 100.

In embodiments, z2 is an integer from 2 to 50. In embodiments, z2 is an integer from 2 to 40. In embodiments, z2 is an integer from 2 to 30. In embodiments, z2 is an integer from 2 to 20. In embodiments, z2 is an integer from or 2 to 10. In embodiments, z2 is an integer from 5 to 100. In embodiments, z2 is an integer from 5 to 30. In embodiments, z2 is an integer from 15 to 100. In embodiments, z2 is an integer from 25 to 100. In embodiments, z2 is an integer from 35 to 100. In embodiments, z2 is an integer from 45 to 100. In embodiments, z2 is an integer from 55 to 100. In embodiments, z2 is an integer from 65 to 100. In embodiments, z2 is an integer from 75 to 100. In embodiments, z2 is an integer from 85 to 100. In embodiments, z2 is an integer from 95 to 100. In embodiments, z2 is 5. In embodiments, z2 is 6. In embodiments, z2 is 7. In embodiments, z2 is 8. In embodiments, z2 is 9. In embodiments, z2 is 10. In embodiments, z2 is 11. In embodiments, z2 is 12. In embodiments, z2 is 13. In embodiments, z2 is 14. In embodiments, z2 is 15. In embodiments, z2 is 16. In embodiments, z2 is 17. In embodiments, z2 is 18. In embodiments, z2 is 19. In embodiments, z2 is 20. In embodiments, z2 is 21. In embodiments, z2 is 22. In embodiments, z2 is 23. In embodiments, z2 is 24. In embodiments, z2 is 25.

In embodiments, z5 is an integer from 1 to 10. In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7. In embodiments, z5 is 8. In embodiments, z5 is 9. In embodiments, z5 is 10.

In some embodiments, a co-oligomer can have any of the foregoing formula in which z5 is an integer from 1 to 3. In some other embodiments, z5 is 1 or 3. In still some other embodiments, z5 is 1. In some still other embodiments, z5 is 3.

In some embodiments, a co-oligomer can have any of the foregoing formula in which R² is hydrogen.

In some embodiments, a co-oligomer can have any of the foregoing formula in which L² is a bond.

In embodiments, a co-oligomer has a formula

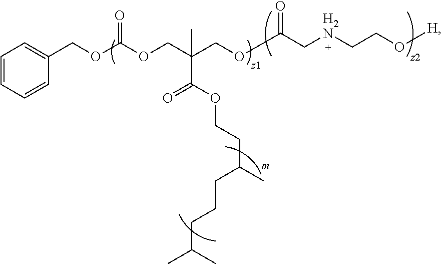

wherein m is 1, 2, or 3;
z1 is an integer from 5 to 30; and
z2 is an integer from 10 to 30.

In embodiments, m is 1. In embodiments, m is 2. In embodiments, m is 3.

In embodiments, z1 is an integer from 5 to 30.

Exemplary co-oligomers include:

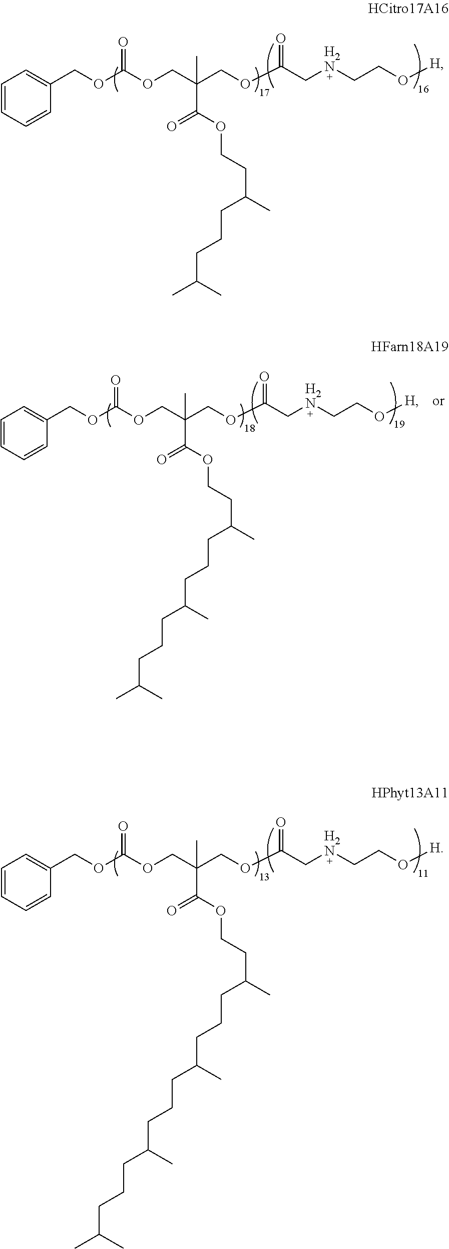

Further to the cell-penetration complex disclosed herein and embodiments thereof, in embodiments, the nucleic acid may be DNA or RNA, such as messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). The cell-penetration complex may further include a protein or peptide.

Further to the cell-penetration complex disclosed herein and embodiments thereof, in embodiments the co-oligomer complexed with nucleic acid further includes a plurality of lipophilic moieties.

Further to the cell-penetration complex disclosed herein and embodiments thereof, in embodiments the co-oligomer complexed with nucleic acid further includes a plurality of immolation domains.

Further to the cell-penetration complex disclosed herein and embodiments thereof, in embodiments, the counter-anion to the above cationic sequences can include common counterions known in the art, such as for example acetate, trifluoroacetate, triflate, chloride, bromide, sulfate, phosphate, succinate, or citrate. In embodiments, the counter-anion is acetate, trifluoroacetate, triflate, chloride, bromide, sulfate, phosphate, succinate, or citrate.

### Methods

The disclosure provides methods of targeted delivery of nucleic acids to particular types of cells and/or tissues in vitro, ex vivo, or in vivo. Any nucleic acid cargo may be ionically complexed to a co-oligomer as described herein for intracellular delivery and release. Accordingly, the disclosure provides methods of transfecting a nucleic acid into a target cell, the methods comprising contacting the target cell with a co-oligomer as disclosed herein complexed with the nucleic acid.

In embodiments, the nucleic acid cargo is RNA or DNA. In embodiments, the RNA is messenger RNA (mRNA), small interference RNA (siRNA), short hairpin RNA (shRNA), micro RNA (miRNA), guide RNA (gRNA), CRISPR RNA (crRNA), or transactivating RNA (tracrRNA). In embodiments, the DNA is plasmid DNA (pDNA), minicircle DNA, or genomic DNA (gNDA).
In embodiments, the nucleic acid cargo is a therapeutic agent, or the nucleic acid encodes one or more therapeutic agents that upon intracellular delivery and release are transcribed into one or more therapeutic agents, such as cytokines or cellular receptors, for example a T cell receptor (TCR) or a chimeric antigen receptor (CAR). In embodiments, the CAR is a Lewis Y antigen CAR (i.e., a chimeric antigen receptor binding to Lewis Y antigen). In embodiments, the CAR is an anti-CD44v6 CAR. In embodiments, the CAR is an anti-NKG2D ligand CAR. In embodiments, the CAR is an anti-folate receptor beta CAR. In embodiments, the CAR is an anti-CD38 CAR. In embodiments, the CAR is an anti-CD20 CAR. In embodiments, the CAR is an anti-CD22 CAR. In embodiments, the CAR is an anti-FLT3 CAR. In embodiments, the CAR is an anti-CD7 CAR. In embodiments, the CAR is an anti-CD33 CAR. In embodiments, the CAR is an anti-CD123 CAR. In embodiments, the CAR is an anti-CLEC12A CAR.

The polyaminoester)s disclosed herein can be utilized as customizable, biodegradable, biocompatible materials for applications in biomedical therapies, imaging and devices. The copolymerization with biodegradable, non-toxic compounds materials such as valerolactone, caprolactone, lactide, and cyclic carbonates allows for tuning physical and biological properties including cargo release rates, hydrophobicity, incorporation of targeting ligands, biodistribution, and toxicity.

Accordingly, in some embodiments, the co-oligomers described here may be derived from cyclic amino-ester and cyclic methyl trimethylene carbonate (MTC) monomers. Cyclic amino-esters have the base structure of morpholin-2-one and homologs thereof, with multiple substitution patterns possible including the following.
(1) N-acylation with a variety of hydrophobic groups (e.g., R=alkyl, alkenyl, aryl, polycycles including steroids, heterocycles), cationic groups (e.g., ammonium, phosphonium, sulfonium, guanidinium, including acylation with amino acids such as glycine, lysine, ornithine, arginine), anionic groups (e.g., carboxylate, sulfate, phosphate), or hydrophilic (e.g., PEG) carbamates. Protection of the morpholine nitrogen with N-Boc or N-Cbz groups followed by organocatalytic ring opening oligomerization or polymerization can afford upon deprotection cationic polymer or oligomer backbones.
(2) Alpha-alkylation or functionalization next to the ester carbonyl with the aforementioned possible functionalities selected to allow for cargo complexation and subsequent cargo release by biodegradation.
(3) Alkylation proximal to the morpholine nitrogen with the aforementioned functionalities.
(4) A combination of the above modifications.

Additionally, copolymers or co-oligomers (block or statistical) can be made by mixing two or more morpholin-2-one monomers, or by the copolymerization (or co-oligomerization) of one or multiple morpholin-2-one monomers with one or multiple cyclic carbonate monomers described herein. These carbonate monomers can incorporate a similar variety of side chain functionality, notably lipophilic groups or cationic groups to modulate oligonucleotide stability, delivery, and release properties. Furthermore, a variety of other commercially available cyclic ester monomers can be used including but not limited to lactide, glycolide, valerolactone, and/or caprolactone to incorporate lipophilic functionality.

The synthesis of poly aminoesters and poly(carbonate-co-aminoester)s is achieved through the ring-opening polymerization and/or copolymerization of morpholine-2-one and cyclic carbonate monomers. The N-Boc protected morpholinone (MBoc) polymerizes to high conversion (>85%), tunable Mn (1 kDa-20 kDa), and low molecular weight distributions (Mw/Mn-1.1-1.3) using an organocatalytic system. Post-polymerization deprotection of the Boc groups affords a cationic (diprotic, secondary amine) water-soluble polymer (−0.5M in D20, stable for >3 days). Furthermore, copolymerization of MBoc with MTC-dodecyl carbonate monomers followed by deprotection give rise to moderately charged cationic materials in high yield (>60%) with narrow polydispersity<1.4 PDI) and tunable block length. Block length is controlled by the ratio of initiator to monomer.

The poly(aminoester)s described here are biocompatible and biodegradable. In certain embodiments, the poly(aminoester)s rapidly degrade through a unique pH-dependent intramolecular rearrangement to generate bis-N-hydroxyethy-2,5-piperizinedionebis-hydroxyethyl glycine (FIGS. 8A-8C). This unique degradation produces a product that is substantially nontoxic when the co-oligomer is administered in a therapeutic amount to a subject. In addition, the monomeric form, which is the expected product of further hydrolysis, can be used as a biomarker for phospholipid modification in the Maillard reaction. The carbonate segment of the poly(aminoester) degrades through hydrolysis and decarboxylation, and its byproducts have previously been shown to be non-toxic.

In embodiments, the methods described here may include complexation of the nucleic acid cargo with a co-oligomer in the presence of a coordinating metal such as Zn⁺², Mg⁺², Ca⁺²; a dynamic non-covalent cross linker such as a carbohydrate; a counterion such as Cl⁻, AcO⁻, succinate, or citrate; or a solubility modulator such as a lipid or a polyethyleneglycol (PEG), or any combination thereof.

In embodiments, a method of transfecting a nucleic acid into a cell as described herein may be part of a method for gene editing or genetic engineering. For example, one or more nucleic acids may be transfected using the co-oligomers described herein in a CRISPR-based system or a transposon-based system for gene editing or genetic engineering. In embodiments, gene editing may result in a DNA deletion, a gene disruption, a DNA insertion, a DNA inversion, a point mutation, a DNA replacement, a knock-in, or a knock-down. In one aspect, the nucleic acid transfected according to the methods described here may comprise one or more vectors having a first nucleotide sequence encoding a CRISPR-Cas system guide RNA that hybridizes with a target sequence in the genome of the cell and a second nucleotide sequence encoding a Cas9 protein. In certain embodiments, the first and second nucleotide sequence can be located on the same or different vectors. In some embodiments, the nucleic acid may comprise a CRISPR RNA (crRNA). In some embodiments, this crRNA can be in the same vector of the first nucleotide sequence encoding a CRISPR-Cas system guide RNA. In some embodiments, the nucleic acid may comprise a transactivating RNA (tracrRNA). In some embodiments, this tracrRNA can be in the same vector of the second nucleotide sequence encoding a Cas9 protein. In some embodiments, the Cas9 protein is codon optimized for expression in the transfected cell.

In another aspect, the nucleic acid may comprise one or more vectors having a first nucleotide sequence encoding a transposase and a second nucleotide sequence having a nucleic acid sequence of a gene of interest flanked by a transposase recognition site. In some embodiments, the first and second nucleotide sequences can be located on the same or different vectors. Transposase generally refers to an enzyme that can bind to a transposon and catalyze the movement of the transposon to another part of the genome by, e.g. a cut and paste mechanism or a replicative transposition mechanism. Introduction of transposase and a gene of interest flanked by a transposase recognition site in cells can induce insertion of the gene of interest into a cellular genome.

In some embodiments, a co-oligomer as described herein complexed with nucleic acid can be used as a vaccine. In some embodiments, a disease or condition that is targeted by the vaccine or vaccine composition can include, but not limited to, an autoimmune, inflammatory, cancer, infectious, metabolic, developmental, cardiovascular, liver, intestinal, endocrine, neurological, or other disease. In some embodiments, the nucleic acid that is contained in the vaccine or composition thereof can be a nucleic acid sequence encoding an antigenic or immunogenic epitope. In embodiments, two separate nucleic acids can encode two different immunogenic peptides. Therefore, in some embodiments, a vaccine composition described here can transfect (1) a first nucleic acid encoding a first immunogenic peptide that can induce more immediate treatment effect to an existing disease or condition and (2) a second nucleic acid encoding a different, second immunogenic peptide that is aimed to induce adaptive immunity in the subject for future occurrence of a different disease or condition. In some embodiments, the vaccine can deliver two or more different nucleic acids to a subject and each nucleic acid independently exhibits a therapeutic or prophylactic effect, respectively.

In embodiments a vaccine composition can have two or more different types of co-oligomer. Alternatively, a vaccine composition can have only a single type of co-oligomer. In some embodiments, a single type of co-oligomer can be non-covalently bound to one type (sequence) of nucleic acid. Alternatively, a single type of co-oligomer can be non-covalently bound to two or more types (sequences) of nucleic acid. Therefore in embodiments, a mixture of different types of co-oligomers, each of which is bound to a different sequence of nucleic acid, can be administered together to a subject in order to deliver two or more sequences (or types) of nucleic acids. Alternatively, a single type (or formula) of co-oligomer that is bound to multiple types (or sequences) of nucleic acid can be administered to a subject in order to deliver two or more sequences (or types) of nucleic acid. Still alternatively, a single type (or formula) of co-oligomer that is bound to a single sequence (or type) of nucleic acid can be administered to a subject.

In some embodiments, the nucleic acid that is contained the vaccine or composition thereof can be messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). In alternative embodiments, the nucleic acid that is contained the vaccine or composition thereof can be mRNA. In some embodiments, nucleic acid is transfected into one or more cells in the subject via vaccination. In some embodiments, one or more than one nucleic acid sequences can be transfected via a vaccine composition. Therefore, in some embodiments, a vaccine composition contains two different nucleic acids, each of which encodes different antigenic peptides. Accordingly, when the vaccine is administered into a subject in need of the vaccination, two or more types of antigenic epitopes can be expressed and induce immune responses in the subject. In alternative embodiments, one type of nucleic acid can be transfected via vaccination such that one type of epitope can be expressed and induce an immune response in the subject.

In embodiments, the nucleic acid includes one or more vectors. In embodiments, the vector may include (a) a first polynucleotide encoding a CRISPR-Cas system guide RNA that hybridizes with a target sequence in the genome of the cell, and (b) a second polynucleotide encoding a Cas9 protein, optionally wherein the Cas9 protein is codon optimized for expression in the cell. In embodiments, the first (a) and second (b) polynucleotides are located in the same or different vectors.

In embodiments, the nucleic acid comprises a CRISPR RNA (crRNA), optionally wherein the crRNA is in the same vector as the first nucleotide sequence.

In embodiments, the nucleic acid comprises a transactivating RNA (tracrRNA). In embodiments, the tracrRNA is optionally in the same vector as the second nucleotide sequence.

In embodiments, the nucleic acid includes (a) a first polynucleotide encoding a transposase; and (b) a second polynucleotide comprising a nucleic acid sequence of a gene of interest flanked by a transposase recognition site. In embodiments, the first (a) and second (b) polynucleotides are located in the same or different vectors. In embodiments, the transposase recognizes and excises a genomic sequence of interest.

### Compounds

Provided herein are compounds that are co-oligomers comprising non-linear branched lipophilic monomers (LP) and poly(alpha-aminoester) monomers (AM) of formula I or I′:

R^{1A}-[L¹-[(LP¹)_{z1}-(LP²)_{z3}-(AM)_{z2}]_{z4}-L²-R^{2A}]_{z5} (I), or

R^{1A}-[L¹-[(LP¹)_{z1}-(AM)_{z2}-(LP²)_{z3}]_{z4}-L²-R^{2A}]_{z5} (I′)
wherein:
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCH₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are optional linkers and are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are same or different;
AM is a poly(alpha-aminoester) monomer;
z5 is an integer from 1 to 10;
z1 and z3 are independently integers from 5 to 30, wherein at least one of z1, or z3 is not 0;
z4 is an integer from 1 to 100, and
z2 is an integer from 5 to 30.

In embodiment, each LP¹ or LP² is independently a non-linear branched lipophilic monomer having a structure of:

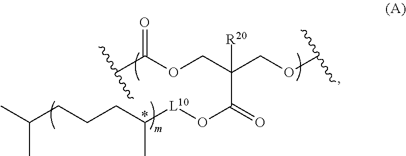

wherein:
m is an integer from 0 to 6;
L¹⁰ is independently a substituted or unsubstituted alkylene; and
R²⁰ is independently hydrogen or unsubstituted C₁-C₄ alkyl.

In embodiments, LP¹ or LP² are independently anon-linear branched lipophilic monomer having a structure of

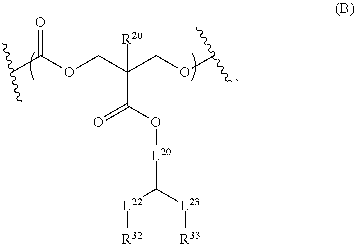

wherein:
L²⁰ is -L^{20A}-L^{20B}-;
each L^{20A} and L^{20B} is independently a bond, a substituted or unsubstituted alkylene, substituted or unsubstituted heteroalkylene, substituted or unsubstituted cycloalkylene, or substituted or unsubstituted heterocycloalkylene;
each L²², and L²³ is independently a bond, substituted or unsubstituted alkylene, or substituted or unsubstituted heteroalkylene;
R²⁰ is independently hydrogen or unsubstituted C₁-C₃ alkyl; and
each R³² and R³³ are independently hydrogen, or substituted or unsubstituted alkyl.

In embodiments, the poly(alpha-aminoester) monomer (AM) has the formula:

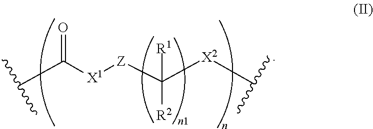

wherein n is an integer of 2 or more as described herein and n1, Z, X¹, X², R¹ and R² are as described herein.

In embodiments, the poly(alpha-aminoester) monomer (AM) has the formula:

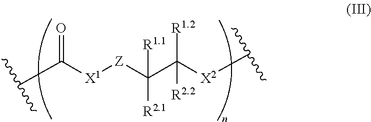

wherein n is an integer of 2 or more as described herein and Z, X¹, X², R^{1.2}, R^{1.2}, R^{2.1} and R^{2.2} are as described herein.

In embodiments, the poly(alpha-aminoester) monomer (AM) has the formula:

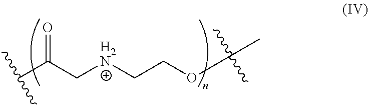

wherein n is an integer of 2 or more as described herein.

In embodiments, the poly(alpha-aminoester) monomer (AM) has the formula:

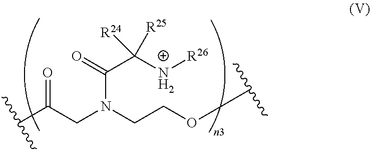

wherein n3 is an integer of 2 or more as described herein, R²⁴, R²⁵ and R²⁶ are as described herein.

In embodiments, the poly(alpha-aminoester) monomer (AM) has the formula:

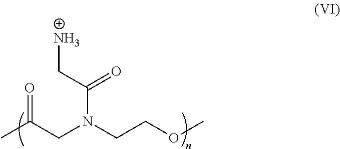

wherein n is an integer of 2 or more as described herein.

In embodiments, the poly(alpha-aminoester) monomer (AM) has the formula:

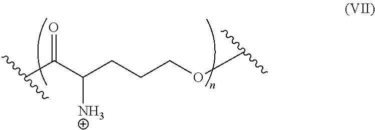

wherein n is an integer of 2 or more as described here.

In accordance with any of the embodiments described herein where n is an integer, n may be an integer in the range 2-100, 2-90, 2-80, 2-70, 2-60, 2-50, 2-40, 2-30, 2-2, or 2-10. In embodiments, n is an integer in the range 2-100 or 2-50.

In embodiments, R^{1A} is a substituted or unsubstituted cycloalkyl, substituted or unsubstituted heterocycloalkyl, substituted or unsubstituted aryl, or substituted or unsubstituted heteroaryl. In embodiments. CART represents the formula of -L¹-[(LP¹)_{z1}-(LP²)_{z3}-(AM)_{z2}]_{z4}-L²-R^{2A} as described above.

In embodiments, the co-oligomer has the formula:

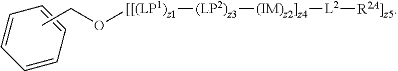

In embodiments the co-oligomer has the formula:

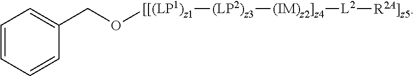

In embodiments, the co-oligomer has the formula:

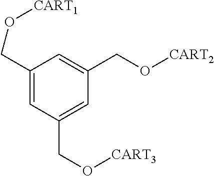

wherein each CART₁, CART₂ and CART₃ independently represents the formula of -L¹-[(LP¹)_{z1}-(LP²)_{z3}-(AM)_{z2}]_{z4}-L²-R^{2A} as described above. L¹, L², R^{2A}, LP¹, LP², AM, z1, z2, z3, z4, and z5 are as described herein.

In embodiments, the nucleic acid is an messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). In embodiments, the nucleic acid includes a sequence encoding a chimeric antigen receptor (CAR).

In an aspect, a nanoparticle composition including a plurality of co-oligomers complexed with nucleic acid as provided herein including embodiments thereof is provided.

In an aspect, a pharmaceutical composition including the complex as provided herein including embodiments thereof and a pharmaceutically acceptable carrier is provided.

In an aspect, a pharmaceutical composition including the nanoparticle composition as provided herein including embodiments thereof and a pharmaceutically acceptable carrier is provided.

In an aspect, a method of transfecting a nucleic acid into a cell ex vivo is provided, the method including contacting a cell with the complex as provided herein including embodiments thereof.

In an aspect, a method of transfecting a nucleic acid into a cell is provided, the method including contacting a cell with the complex as provided herein including embodiments thereof.

In an aspect, a method of transfecting a nucleic acid into a cell ex vivo is provided, the method including contacting a cell with the nanoparticle composition as provided herein including embodiments thereof.

In an aspect, a method of delivering a nucleic acid to a cell in a subject is provided, the method including administering the pharmaceutical composition as provided herein including embodiments thereof.

In embodiments, the co-oligomer is allowed to degrade within the cell thereby forming a degradation product. In embodiments, the degradation product is a substituted or unsubstituted diketopiperazine.

In embodiments, the methods further include allowing the mRNA to be expressed in the cell. In embodiments, the cell is an eukaryotic cell. In embodiments, the cell is a mammalian or human cell. In embodiments, the cell forms part of an organism. In embodiments, the organism is a human. In embodiments, the cell is a lymphoid cell or a myeloid cell. In embodiments, the cell is a T cell. In embodiments, the cell is a myeloid cell.

In an aspect is provided, a method of inducing an immune response in a subject in need thereof, the method including administering an effective amount of the complex as provided herein including embodiments thereof. In embodiments, the immune response is an anti-cancer immune response.

In an aspect, a method of transfecting a nucleic acid encoding a chimeric antigen receptor (CAR) into a cell is provided. The method includes contacting a cell with a co-oligomer complexed with nucleic acid non-covalently bound to a co-oligomer, the co-oligomer including a poly(alpha-aminoester) monomer and a non-linear branched lipophilic monomer, and the nucleic acid including a sequence encoding a chimeric antigen receptor.

### Non-Linear Branched Lipophilic Monomers

In an aspect is provided a non-linear branched lipophilic monomer that is polymerized to form a co-oligomer with an alpha(aminoester) as described herein.

In embodiments, the non-linear branched lipophilic monomer has a structure of

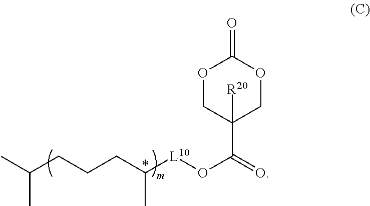

m, L¹⁰, and R²⁰ are as described herein.

In embodiments, the non-linear branched lipophilic monomer having a structure of:

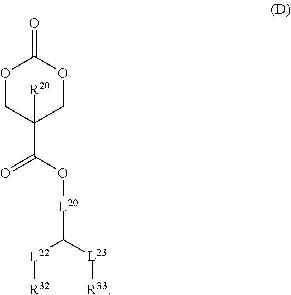

L²⁰, L²², L²³, R²⁰, R³², and R³³ are as described herein.

In embodiments, the non-linear branched lipophilic monomer having a structure of:

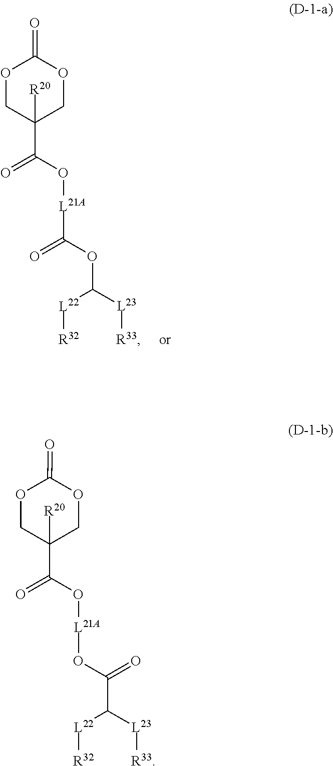

L^{21A}, L²², L²³, R²⁰, R³², and R³³ are as described herein.

In embodiments, the non-linear branched lipophilic monomer having a structure of:

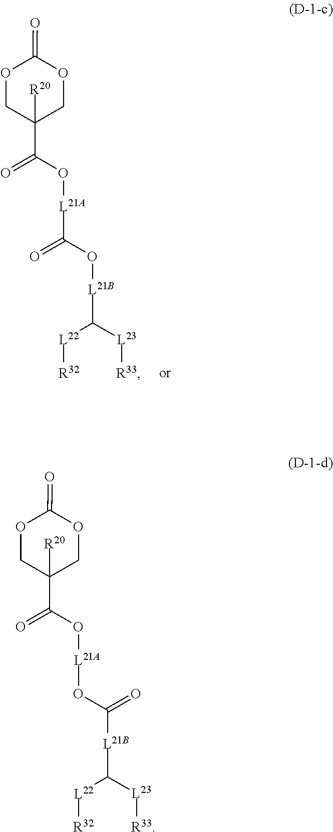

L^{21A}, L^{21B}, L²², L²³, R²⁰, R³², and R³³ are as described herein.

In embodiments, the non-linear branched lipophilic monomer having a structure of:

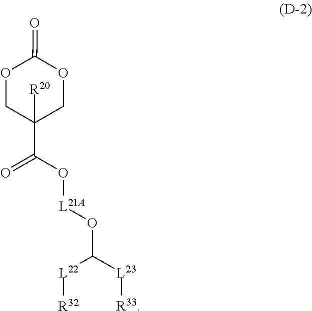

L^{21A}, L²², L²³, R²⁰, R³², and R³³ are as described herein.

In embodiments, the non-linear branched lipophilic monomer having a structure of

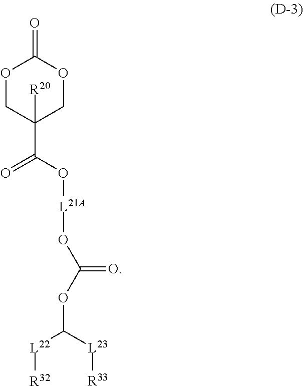

L^{21A}, L²², L²³, R²⁰, R³², and R³³ are as described herein.

In embodiments, the non-linear branched lipophilic monomer having a structure of:

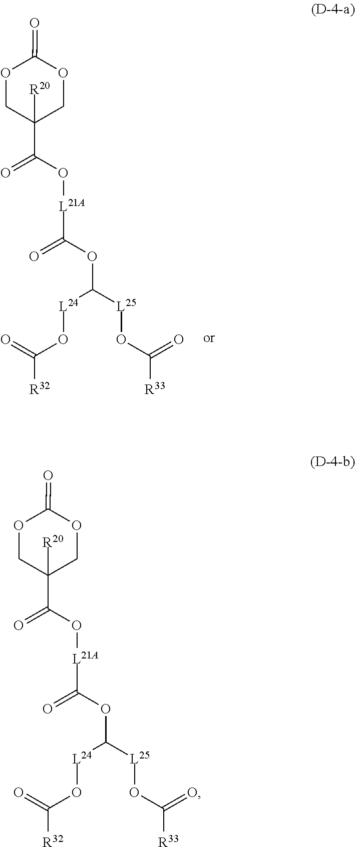

L^{21A}, L²⁴, L²⁵, R²⁰, R³², and R³³ are as described herein.

In embodiments, the non-linear branched lipophilic monomer having a structure of

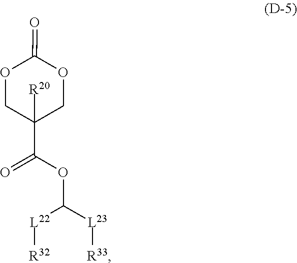

wherein. L²², L²³, R²⁰, R³², and R³³ are as described herein.

In embodiments, L²² is independently a bond. In embodiments, L²³ is independently a bond. In embodiments, the non-linear branched lipophilic monomer has a structure of

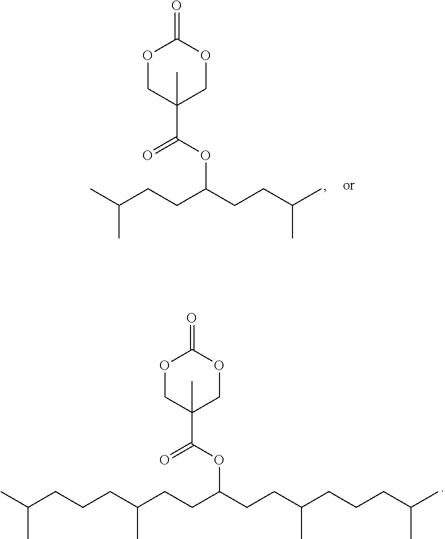

In embodiments, the non-linear branched lipophilic monomer having a structure of

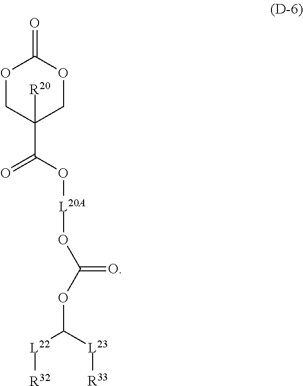

wherein L^{20A} is substituted or unsubstituted cycloalkylene, or substituted or unsubstituted heterocycloalkylene. R²⁰, R³², R³³, L²² and L²³ are as described herein.

In embodiments, the non-linear branched lipophilic monomer has a structure of

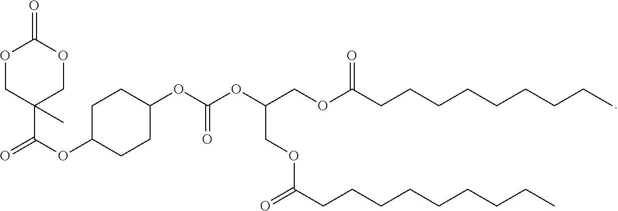 [SMILES: CCCCCCCCCC(=O)OCC(COC(=O)CCCCCCCCC)OC(=O)OC1CCC(OC(=O)C2(C)COC(=O)OC2)CC1]

In embodiments, the non-linear branched lipophilic monomer has a structure of

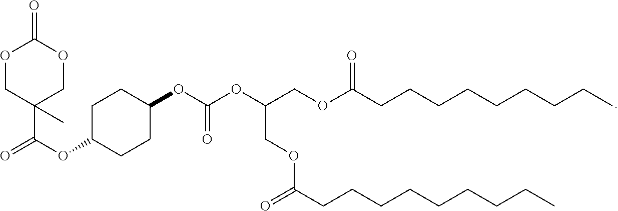

In embodiments, the non-linear branched lipophilic monomer has a structure of

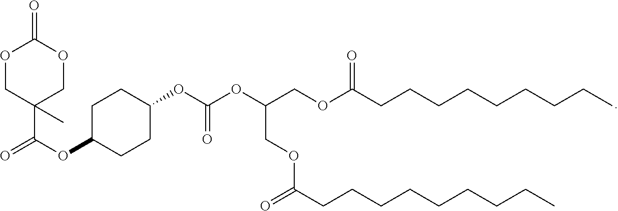

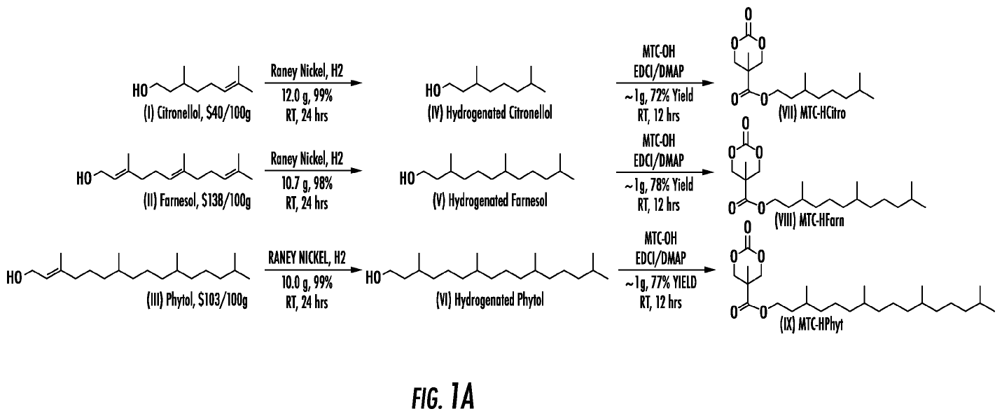

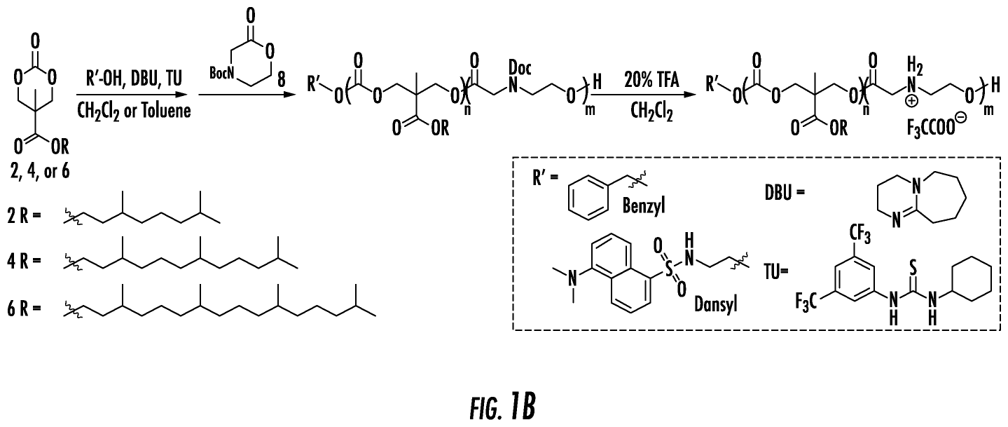

In embodiments, the non-linear branched lipophilic monomer is methyl-trimethylenecarbonate (MTC) monomers, for example, as shown in FIGS. 1-3, 6A and 6B. In embodiments, the methyl-trimethylenecarbonate (MTC) monomers include MTC-C6-dibutyl methanol. MTC-C6-dlhexyl methanol, MTC-C6-diisobutyl methanol, MTC-C6-isobutylnonenyl carbinol, MTC-C6-butyloctanoate, MTC-C6-dlibutyl methanol ether, MTC-C6-dlnonenyl methanol, MTC-C6-dicitronellyl methanol, MTC-C6-dilsoamylimethanol, MTC-C6-hexyldecanoate, MTC-C6-dloctyl methanol, MTC-C6-dibutyl-methanol carbonate, MTC-C6-gly-1,3-dilauryl, MTC-C6-gly-1,3-dimyristyl, MTC-C6-gly-1,3-dipalmityl, MTC-C6-gly-1,3-dihexyl, MTC-C6-gly-1,3-didecyl, or MTC-C6-gly-1,3-dicitronellyl.

### Pharmaceutical Compositions

The disclosure also provides pharmaceutical compositions comprising a co-oligomer as described herein, which can be used for therapy. In some embodiments, the co-oligomer is complexed with a nucleic acid. In embodiments, the composition has a co-oligomer but not a cargo nucleic acid. In accordance with these embodiments, the cargo nucleic acid can be complexed with the co-oligomer before administration of the composition to a subject.

In some embodiments, a composition can be a vaccine or a composition thereof, i.e. a composition that contains the vaccine and optionally a pharmaceutically acceptable carrier. The vaccine or vaccine composition can be used to prevent and/or treat a disease or condition or a pathogen associated with the disease or condition. In some embodiments, the vaccine or vaccine composition contains a co-oligomer and a cargo nucleic acid. In some embodiments, the co-oligomer complexed with nucleic acid, when administered to a subject, can induce an immune response, i.e. immunogenic. This immunogenicity can be induced, at least in part, when one or more antigenic peptides encoded by the cargo nucleic acid are expressed in the transfected cells.

In some embodiments, pharmaceutical compositions may contain pharmaceutically acceptable excipients or additives depending on the route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used can be chosen from, but not limited to, the above or combinations thereof, as appropriate, depending on the dosage form of the present disclosure.

In some embodiments, the pharmaceutically acceptable carrier is an immunological adjuvant. In embodiments, the immunological adjuvant can include, but is not limited to, agonists of Toll-like Receptors (TLRs), agonists of the STING pathway, agonistic antibodies against CD40, OX40, CTLA4, PD1, or PD1-L, Freund's adjuvant, bryostatins and ligands for CD40, OX40, CD137, PD1, CTLA4 and any combinations thereof. In some embodiments, the adjuvant can increase immunogenicity that is induced when a co-oligomer complexed with nucleic acid by co-administered with the complex to a subject.

Formulation of the pharmaceutical compositions of the present disclosure can vary according to the route of administration selected (e.g., solution, emulsion). Routes of administration can be, for example, intramuscular, subcutaneous, intravenous, intralymphatic, subcutaneous, intramuscular, intraocular, topical skin, topical conjunctival, oral, intravessical (bladder), intraanal and intravaginal.

In some embodiments, the composition can include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition into a matrix; enteric coatings; micro-encapsulation; gels and hydrogels; implants; and any other formulation that allows for controlled release of a composition.

In one aspect is provided a kit of parts having a co-oligomer complexed with nucleic acid or composition thereof. In another aspect is provided a kit of parts having a co-oligomer that is not bound to a nucleic acid or composition thereof. The kit can further contain a document or an instruction that describes a protocol for making a co-oligomer complexed with nucleic acid. The document or instruction of the kit can also describe a protocol for administering the composition to a subject in need thereof.

Therapeutic formulations described herein can be prepared for storage by mixing the active ingredients, i.e., immunogenic agent(s) having the desired degree of purity with optional physiologically acceptable carriers, excipients or stabilizers. Acceptable carriers, excipients, or stabilizers can be nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound (e.g., a second active agent in addition to the immunogenic agent(s) that has a co-oligomer complexed with nucleic acid), which may be selected for complementary activities that do not adversely affect each other. Such molecules can be suitably present in combination in amounts that can be effective for the purpose intended.

### Administration

In some aspects provided are methods for delivering a composition to cells or a subject so as to provide a desired activity into the cells or subject. In some embodiments, the composition can contain a co-oligomer complexed with nucleic acid where a cargo nucleic acid is non-covalently bound to a co-oligomer. The cargo nucleic acid, when transfected into the cells or administered to the subject, can provide a variety of intended effects, depending on the nature of the nucleic acid sequence. Some non-limiting examples of intended effects include modulation on gene expression, modulation of cellular pathways, genome-edition and induction of an immune response. In some embodiments, the composition can be administered to a subject in an effective amount that is sufficient to achieve at least part of the intended effects in the subject.

“Administration,” “administering” and the like, when used in connection with a composition refer both to direct administration, which may be administration to cells in vitro, administration to cells in vivo, administration to a subject by a medical professional or by self-administration by the subject and/or to indirect administration, which may be the act of prescribing a composition of the disclosure. When used herein in reference to a cell, refers to introducing a composition to the cell. Typically, an effective amount is administered, which amount can be determined by one of skill in the art. Any method of administration may be used. Compounds (e.g., drugs and antibodies) may be administered to the cells by, for example, addition of the compounds to the cell culture media or injection in vivo. Administration to a subject can be achieved by, for example, intravascular injection, direct intratumoral delivery, and the like.

Administering may mean oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the disclosure can be administered alone or can be co-administered to the patient. Co-administration is meant to include simultaneous or sequential administration of the compounds individually or in combination (more than one compound).

The dosage and frequency (single or multiple doses) administered to a subject can vary depending upon a variety of factors, for example, whether the subject suffers from another disease, its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

In some embodiments, the subject is a mammal, for example a human, anon-human primate, a murine (i.e., mouse and rat), a canine, a feline, or an equine. In embodiments, the subject is a human.

In some embodiments, a composition can be administered in a dose (or an amount) of about 1 ng/kg of subject body weight, about 10 ng/kg of subject body weight, about 50 ng/kg of subject body weight, about 100 ng/kg of subject body weight, about 500 ng/kg of subject body weight, about 1 μg/kg of subject body weight, about 10 μg/kg of subject body weight, about 50 μg/kg of subject body weight, about 100 μg/kg of subject body weight, about 150 μg/kg of subject body weight, about 200 μg/kg of subject body weight, about 250 μg/kg of subject body weight, about 300 μg/kg of subject body weight, about 350 μg/kg of subject body weight, about 375 μg/kg of subject body weight, about 400 μg/kg of subject body weight, about 450 μg/kg of subject body weight, about 500 μg/kg of subject body weight, about 550 μg/kg of subject body weight, about 600 μg/kg of subject body weight, about 650 μg/kg of subject body weight, about 700 μg/kg of subject body weight, about 750 μg/kg of subject body weight, about 800 μg/kg of subject body weight, about 850 μg/kg of subject body weight, about 900 μg/kg of subject body weight, about 1 mg/kg of subject body weight, about 10 mg/kg of subject body weight, about 50 mg/kg of subject body weight, about 100 mg/kg of subject body weight, about 500 mg/kg of subject body weight, about 1 μg/kg of subject body weight or more or any intervening ranges of the of the foregoing. In some embodiments, a composition can be administered in a dose (or an amount) of about 0.5 μg, about 1.0 μg, about 1.5 μg, about 2.0 μg, about 2.5 μg, about 3.0 g, about 3.5 μg, about 4.0 μg, about 4.5 μg about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 1.0 mg, about 1.5 mg, about 2.0 mg, about 2.5 mg, about 3.0 mg, about 3.5 mg, about 4.0 mg, about 4.5 mg about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 1 g or more or any intervening ranges of the foregoing. In some embodiments, a composition can be administered in a dose (or an amount) of about 7.5 μg or about 0.375 mg/kg of subject body weight. Administration can be repeated over a desired period, e.g., repeated over a period of about 1 day to about 5 days or once every several days, for example, about five days, over about 1 month, about 2 months, etc. The weight herein can be a weight of a co-oligomer complexed with nucleic acid or a weight of a composition or pharmaceutical formulation thereof.

In embodiments, a composition can be administered intravenously, subcutaneously or intratumorally. In embodiments, a formulation or a pharmaceutical composition can be administered intravenously, subcutaneously or intratumorally.

In embodiments, a composition can be administered systemically or locally (e.g. intratumoral injection, intravenous injection) at intervals of 6 hours, 12 hours, daily or every other day or on a weekly or monthly basis to elicit the desired benefit or otherwise provide a therapeutic effect.

In embodiments, a response rate to a composition, in particular a cancer vaccine, can be reduced as compared to baseline reference or control reference. The term “response rate” is used herein in its customary sense to indicate the percentage of patients who respond with cancer recession following treatment. Response rates include, for example, partial or complete recession. A partial response includes an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, or about 99% recession of cancer cells. In some embodiments, the control reference is obtained from a healthy subject, a cancer subject (e.g., the cancer subject being treated or another cancer subject), or any population thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: General Methods and Experimental

### Materials

Reagents were purchased from Sigma-Aldrich and used as received unless otherwise indicated. 1-(3,5-Bis-trifluoromethyl-phenyl)-3-cyclohexyl-thiourea (Macromolecules 39(23):7863-7871), MTC-guanidine monomer (J Am Chem Soc 131(45):16401-16403), MTC-dodecyl monomer (Proc Natl Acad Sci 109(33):13171-13176), MTC-piperidine monomer (Chem Commun (1):114-116), N-Boc morpholinone monomer (J Am Chem Soc 136(26):9252-9255), and dansyl alcohol (J Am Chem Soc 131(45):16401-16403) were all prepared according to literature procedures. Unless otherwise noted, all commercial solvents and reagents were used without further purification. Methylene chloride (CH₂Cl₂) and tetrahydrofuran (THF) were passed through an alumina drying column (Solv-tek Inc.) using nitrogen pressure. Petroleum ether, pentane, hexane, ethyl acetate (EtOAc), and methanol (MeOH) were obtained from Fisher Scientific. Deuterated solvents were purchased from Cambridge Isotope Laboratories. Regenerated cellulose dialysis membranes (Spectra/Por® 6 Standard RC; MWCO 1000) were purchased from Spectrum Laboratories, Inc.

mRNAsg

In all following examples, eGFP mRNA (5meC, Ψ, L-6101), Fluc mRNA (5meC, Ψ, L-6107), OVA mRNA (5meC, Ψ, L-7210) and Cy5-eGFP mRNA (5meC, Ψ, L-6402) were purchased from TriLink BioTechnologies Inc.

### Instrumentation

Particle size was measured by dynamic light scattering on a Malvern Zetasizer Nano ZS90. Flow cytometry analysis was performed on a BD LSRII FACS Analyzer (Stanford University Shared FACS Facility). Laser scanning confocal microscopy was carried out using a Leica SP8 White Light Confocal microscope with a 40×HC PL APO, CS2 oil objective lens (Stanford University Cell Sciences Imaging Facility). Bioluminescence was measured using a charge-coupled device (CCD) camera (IVIS 100, Xenogen Corp., Alameda, CA) and analyzed using Living Image Software (Perkin-Elmer). Epifluorescence microscopy was performed on a Zeiss Axio Observer.Z1 with an X-Cite 120Q wide-field excitation light source and a GFP filter set. Images were acquired with a CoolSNAP HQ² camera and transferred to a computer for image analysis.

### Cell Lines

HeLa, J774, HepG2, and HEK-293 cells were maintained in Dulbecco's Modified Eagle Medium (DMEM) supplemented with 10% fetal bovine serum (FBS) and 1% penicillin/streptomycin. CHO cells were maintained in F12 media supplemented with 10% FBS and 1% penicillin/streptomycin. All cells were grown at 37° C. in a 5% CO₂ atmosphere. Cells were passaged at approximately 80% confluence.

Mesenchymal stem cells (MSCs) were prepared according to the method of Huang et al (J. Orthopaedic Trans. 3(1):26-33). Briefly, femurs were excised from two 8-week-old female CD1 mice, and the tissue was removed from the outside of the bone. The ends of the bones were then cut with a sterile scissors. The marrow was flushed from the four bones with DMEM 10% fetal calf serum containing penicillin/streptomycin using a 3 mL syringe and a 25 g needle in a 10 cm tissue culture treated petri dish. The marrow was disrupted and dispersed by pipetting, but not filtered or otherwise manipulated. The dish was incubated for 6 days, whereupon a characteristic monolayer developed. The culture was then washed twice with PBS, and trypsinized with 0.25% trypsin (Gibco) for 5 min at 37° C. The cells were then collected and transferred to a 75 cm² tissue culture flask, and incubated for 3 days, until 90% confluence was achieved. The culture could be maintained for two more passages, but growth was greatly reduced upon four passages. For transfection, the cells were plated at 1.2×10⁴ per well in 24-well plates.

In Vivo mRNA Delivery and Flow Cytometry

CART-mRNA polyplexes were prepared as described above using 5 g mRNA per mouse in a total volume of 100 μL. Complexes were mixed for 20 s before i.v. injection into tail vain of female BALB/c mice. For Fluc expression: After 7 h, 150 mg/kg D-luciferin was injected i.p. and luminescence was measured using an IVIS Imaging System CCD camera and analyzed using living image software. For T cell isolation from spleenocyte, animals were killed and single-cell suspensions were prepared by passing the spleens through 70-μm cell strainers followed by lysis of blood cells with ACK Lysing Buffer and washing two times with PBS. T cells were isolated using pan T cell isolation kit (Miltney Biotec) according to manufacture protocol. For CAR19 expression in spleenocytes after CAR19 mRNA-CART treatment, Spleenocytes were partitioned into two staining groups and stained fluorescent antibodies for CD8 (APC), CD4 (PE), and B220 (PerCp) or CD49d(APC). CAR19 construct was detected using a mouse anti-rat kappa chain antibody conjugated to FITC. Analysis was ddone on LSR-II.UV (BD Biosciences) using the Pacific Blue channel. Experimental protocols were approved by the Stanford Administrative Panel on Laboratory Animal Care.

### In Vivo Bioluminescence

i.p. injections of d-Luciferin (Biosynth AG) were done at a dose of 150 mg/kg, providing a saturating substrate concentration for Fluc enzyme (luciferin crosses the blood-brain barrier). Mice were imaged in a light-tight chamber using an in vivo optical imaging system (TVIS 100; Xenogen Corp.) equipped with a cooled charge-coupled device camera. During image recording, mice inhaled isofluorane delivered via a nose cone, and their body temperature was maintained at 37° C. in the dark box of the camera system. Bioluminescence images were acquired between 10 and 20 minutes after luciferin administration. Mice usually recovered from anesthesia within 2 minutes of imaging.

### Mice and Cells Lines

Eight- to twelve-week-old female Balb/c mice were purchased from The Jackson Laboratory and housed in the Laboratory Animal Facility of the Stanford University Medical Center. All experiments were approved by the Stanford Administrative Panel on Laboratory Animal Care and conducted in accordance with Stanford University Animal Facility and NIH guidelines.

Intra-Tumoral Treatment of Established Tumors Using CAR19 mRNA-CART

CD19 expressing tumors were implanted subcutaneous on the right side of the abdomen. Treatment began when tumors reached 7 mm to 10 mm in largest diameter. 5 ug CAR19 mRNA-CART were injected intratumorally into the tumor. Tumor size was monitored with a digital caliper (Mitutoyo) every 2 to 3 days and expressed as volume (length×width×height). Mice are sacrificed when tumor size reached 1.5 cm in the largest diameter as per guidelines.

IV Treatment of Established CD19 Expressing and CD19negative Tumors Using CAR19 mRNA-CART

CD19 expressing and CD19 negative tumors were implanted subcutaneous on the right side of the abdomen. Treatment began when tumors reached 7 mm to 10 mm in largest diameter. 5 ug CAR19 mRNA-CART were injected intravenously. Tumor size was monitored with a digital caliper (Mitutoyo) every 2 to 3 days and expressed as volume (length×width×height). Mice are sacrificed when tumor size reached 1.5 cm in the largest diameter as per guidelines.

### Statistical Analysis

Prism software (GraphPad; La Jolla, CA) was used to analyze tumor growth and to determine statistical significance of differences between groups by applying an non-parametric Mann-Whitney U test. P values<0.05 were considered significant. The Kaplan-Meier method was employed for survival analysis.

### Example 2: Synthesis of Poly(Alpha-Aminoester) Monomers

In some examples, CARTs of varied cationic immolative domains are generated from new lactone or carbonate monomers. Examples of synthesis of precursors and monomers are described as follows:

### Synthesis of tert-butyl 2-oxomorpholine-4-carboxylate (N-Boc morpholinone)

In these examples, synthesis of the N-Boc morpholinone monomer was adapted from the literature: Chung, et al. J. Am. Chem. Soc., 2013, 135 (20), 7593-7602

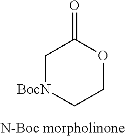 [SMILES: CC(C)(C)OC(=O)N1CCOC(=O)C1]

Synthesis of tert-butyl 2-oxomorpholine-4-carboxylate: 2.086 g (10.2 mmol) tert-butyl bis(2-hydroxyethyl)carbamate was dissolved in 75 mL CH₃CN and bubbled with oxygen for 5 minutes. 398 mg (0.38 mmol) of [(neocuproine)Pd(OAc)]₂(OTf)₂ was added, resulting in the formation of a dark red solution. The reaction was placed in an oil bath at 60° C. and oxygen was bubbled through the reaction mixture, monitoring by TLC (1:1 hexanes:EtOAc). After 24 hours 263 mg (0.25 mmol) of [(neo)Pd(OTf)(Ac)]2 was added to the reaction. After 48 hours an additional 426 mg (0.41 mmol) of [(neocuproine)Pd(OAc)]2(OTf)₂ was added to the reaction. After a total of 72 hours oxygen flow was stopped and the reaction was allowed to cool to room temperature. The reaction mixture was concentrated to 5-10 mL, resulting in a black non-viscous mixture and loaded onto a plug of SiO₂, eluting with 1:1 hexane:EtOAc. Concentration afforded 1.766 g yellow oil (8.78 mmol, 86.4%), which solidified on standing. This crude product was dissolved in 10 mL Et₂O followed by addition of 20 mL hexanes and stored at −30° C. overnight. Precipitate was collected by filtration, washing with pentane, to afford 1.54 g white powder (7.67 mmol, 76% yield). ¹H NMR (500 MHz, CDCl₃): δ 4.41 (br, 2H), 4.26 (s, 2H), 3.66 (t, 2H), 1.47 (s, 9H). ¹³C NMR (125 MHz, CD₃Cl): δ 28.24, 40.63, 45.49, 67.19, 81.33, 153.49, 166.63. HRMS (m/z): M+ calc'd for C₉H₁₅NO₄Na, 224.0893; Found, 224.0896.

### Synthesis of tert-butyl (2-oxo-2-(2-oxomorpholino)ethyl)carbamate(Glycine monomer)

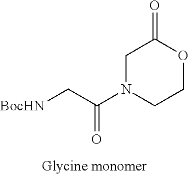 [SMILES: CC(C)(C)OC(=O)NCC(=O)N1CCOC(=O)C1]

A flame dried flask was charged with diol (450 mg, 1.72 mmol) and acetonitrile (12 mL) then bubbled with air for 5 minutes. Pd(Neo)OAc (84.1 mg, 0.16 mmol, 9.4 mol %) was added to the reaction and allowed to stir at 50 C under constant air flow. After 18 hours the reaction was concentrated, triturated with EtOAc (30 mL) then filtered to yield 220 mg foamy orange residue. Silica gel chromatography was performed, eluting with 100% EtOAc. Concentration of the relevant fractions afforded 53.1 mg (0.205 mmol, 12% yield) of a clear oil, which solidified upon standing. ¹H NMR (500 MHz, CD₃Cl): δ 5.45-5.3 (br, 1H), 4.5-4.45 (q, 2H), 4.28-4.4 (d, 2H), 4.0-3.93 (dd, 2H), 3.85-3.7 (dt, 2H), 1.45 (s, 9H) ¹³C NMR (125 MHz, CD₃Cl): δ 167.7, 166.1, 164.8, 155.8, 80.2, 66.9, 65.9, 45.8, 44.1, 42.4, 41.2, 39.3, 28.3, 15.316

### Synthesis of Tert-Butyl (2-Oxotetrahydro-2H-pyran-3-yl)carbamate (Mglut)

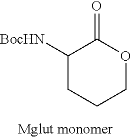 [SMILES: CC(C)(C)OC(=O)NC1CCCOC1=O]

Synthesis of Tert-Butyl (2-Oxotetrahydro-2H-pyran-3-yl)carbamate (Mglut): 1.078 g (4.9 mmol) (S)-(−)-2-(Boc-amino)-1,5-pentanediol was dissolved in 10 ml CH₃CN (0.49 M) and bubbled with air for 5 minutes. 0.233 g (0.45 mmol) of [(neo)Pd(OTf)(OAc)]2 was added, turning the clear solution orange, which darkened to black. The reaction was placed in an oil bath at 45° C. and air was bubbled through the reaction mixture. The reaction was monitored by 1H NMR, looking for disappearance of the lactol peak at 5.1 ppm. Over the course of 44 hours a total of 0.310 g (0.6 mmol, 12 mol %) of [(neo)Pd(OTf)(OAc)]2 was added in portions to the reaction, based on reaction progress. At 44 hours ¹H NMR showed the reaction was complete. Airflow was stopped and the reaction was allowed to cool to room temperature. The reaction mixture was concentrated and the residue taken up in 50 mL EtOAc and sonicated, then filtered through a plug of celite then concentrated, yielding 0.940 g pink-orange oil, which solidified on standing. Silica gel chromatography was performed, eluting with 4:1 DCM:EtOAc. Concentration of the relevant fractions afforded 0.480 g (2.23 mmol) of white solid (46% yield). Spectral data is consistent with previous report. Xie, X. and Stahl, S. S. J. Am. Chem. Soc., 2015, 137 (11): 3767-3770

Preparation of other poly(alpha-aminoester) monomers for CARTs are described previously (e.g., WO2018/022930A1 and WO2020/097614A2).

### Example 3: Non-Linear Branched Lipophilic Monomer

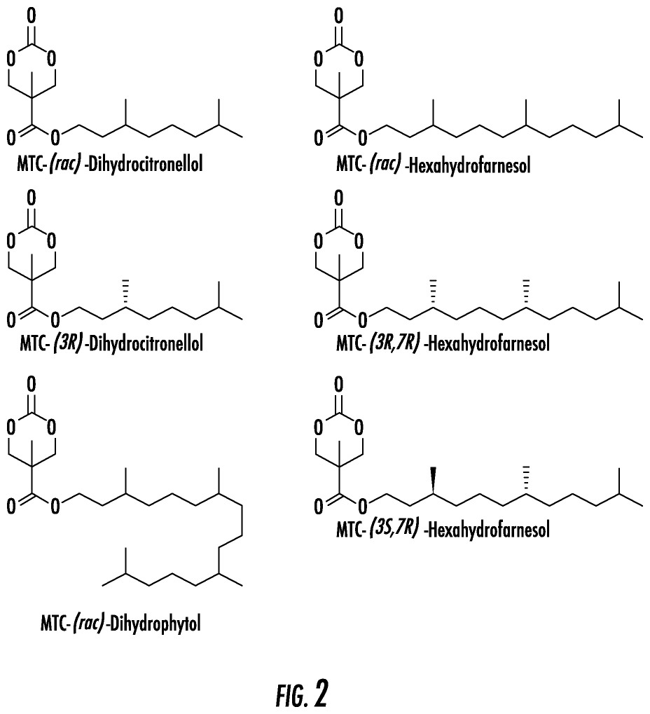

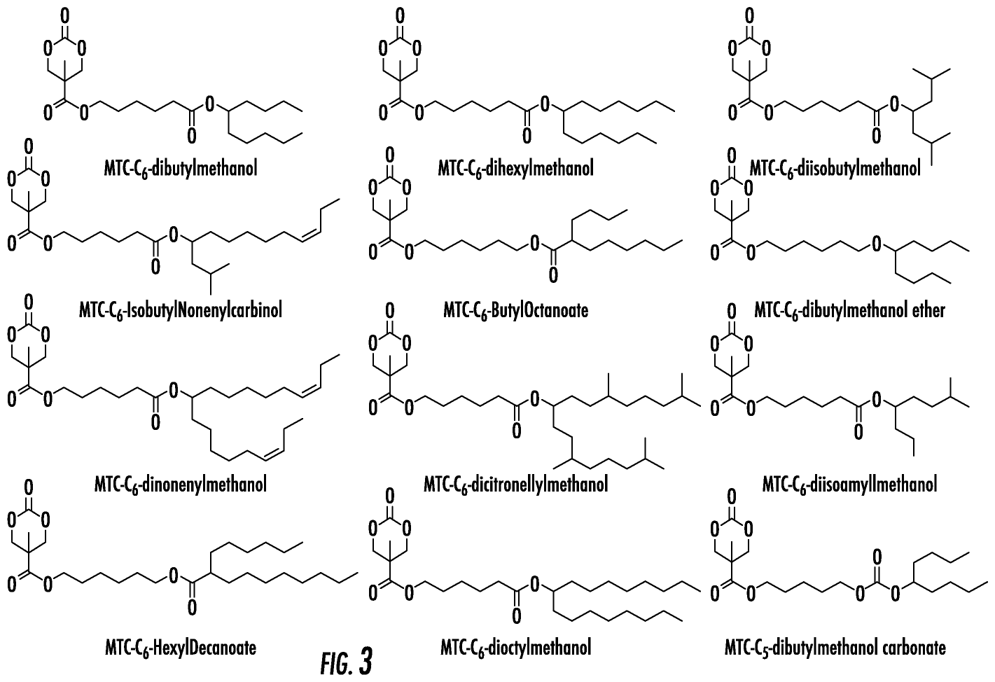

Isoprenoid-based lipid monomers were synthesized in two steps based on common branched lipids found in nature. Lipids were hydrogenated in exceptional yields (>98%) on decagram scale using a common hydrogenating catalyst. Methyl-trimethylenecarbonate (MTC) monomers were synthesized via Steiglich-type coupling to MTC-OH. This precursor was synthesized via the newly reported carbonyl diimidazole cyclization procedure as shown in FIG. 1. Example monomers of each class are shown in FIGS. 2-3.

### Example 4: Polymerization of CARTs

Preparation of oligonucleotide cargos (e.g., siRNA, miRNA, minicircle DNA, pDNA, or Minicircle-DNA), initiators, salts, and counterions for the polymerization of CARTs are described previously (e.g., WO2018/022930A1 and WO2020/097614A2).

### General Oligomerization Procedures

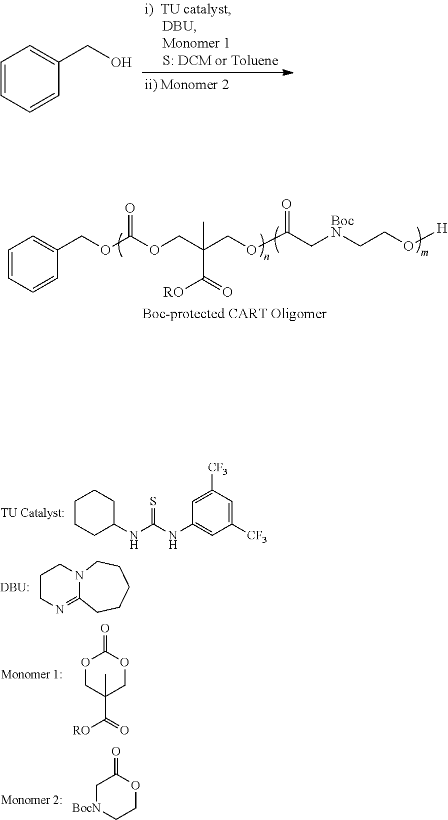

FIG. 1B shows that monomers 2, 4, or 6 are oligomerized via OROP with the DBU/TU catalyst system using a primary alcohol as initiator. In the same vessel, the resulting oligo-carbonate macro-initiator begins the oligomerization of 8. The resulting di-block co-oligomer is purified by dialysis and deprotected with trifluoroacetic acid to yield the active isoprenoid CART in two steps.

A glass GPC vial was charged with a small stir bar, about 0.1 mmol of the desired MTC monomer and 5 mol % of thiourea catalyst. This vial was loosely sealed and placed under high-vacuum overnight. The monomer and catalyst were dissolved in a 0.1 M solution of benzyl alcohol (unless otherwise noted) in Sure-Seal® toluene or distilled dichloromethane and sealed. An argon balloon was affixed to the vial with a 21-gauge needle and a 25-gauge vent needle was added. After five minutes the balloon and vent needle were removed. 1 drop of DBU was added using a 23-gauge needle, and the reaction was stirred under ambient conditions for 2 hours. The vial was carefully opened to atmosphere and solid N-Boc morpholinone monomer (22 mg, 0.11 mmol) was added to the reaction. The reaction was sealed, and an argon balloon was affixed to the vial with a 21-gauge needle and a 25-gauge vent needle was added. After five minutes the balloon and vent needle were removed. The reaction was stirred for an additional 2 hours before quenching the catalyst with 2 drops of acetic acid. The crude reaction solution was diluted with DCM (2 mL) and dialyzed against MeOH (700 mL, 2.0 kDA dialysis bag) for 4 hours with one solvent change at 2 hours. Concentration afforded the protected diblock co-oligomer as a clear oil (yield 67-88% by mass).

Degree of polymerization was determined by ¹H-NMR end group analysis in CDCl₃. Molecular weight distributions (M_{w}/Mₙ; polydispersity index, PDI) was determined by gel permeation chromatography (GPC) with the sample at 3-5 mg/mL in THF.

### General Oligomer Deprotection and Solution Preparation

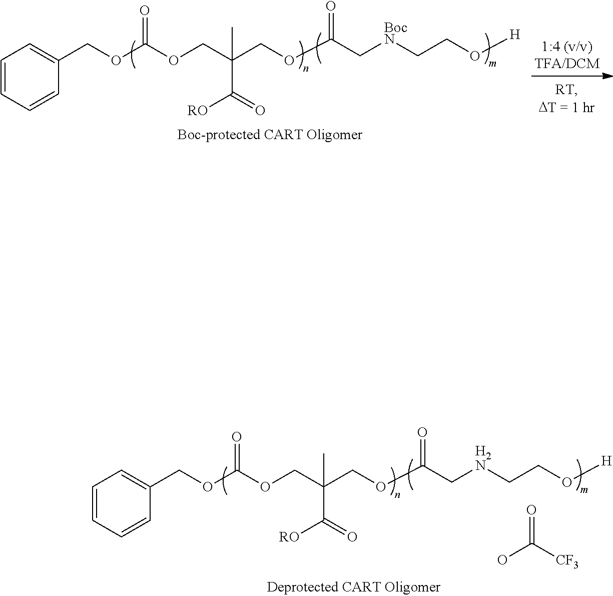

In a 1-dram vial, oligomers (0.5 gmol) were deprotected in a trifluoracetic acid/distilled DCM solution (1:4 v/v, 500 μL) under light stirring and ambient atmosphere for 1 hour. Solvent was removed in vacuo and the samples were stored under high-vacuum for 18 hours. The deprotected oligomer, as a thin film, was taken up in d₆-DMSO (250 μL, Cambridge Isotope Laboratories) with vigorous vortexing. The solution was then split into two ½-dram vials, topped with a blanket of argon and sealed with parafilm before storage at −20° C. until use.

### Example 5: Isoprenoid Based Lipids in Charge-Altering Releasable Transporters (CARTs)

Branched lipids, like linear lipids, have a linear all carbon backbone but the former have one or more carbons connected to that linear backbone branching off like limbs from the trunk of a tree. These branches may be comprised of one or more carbons or other atoms. Branched lipids with methyl group branching are found in nature in the form of isoprenoid lipids and some are components of membranes of cells (archaea). It is hypothesized that branched lipids will pack differently relative to linear lipids and that in turn will affect the physical properties (viscosity, packing, stability, shape, surface area, membrane association, etc) of complexes, particles and membranes formed with branched lipid systems.

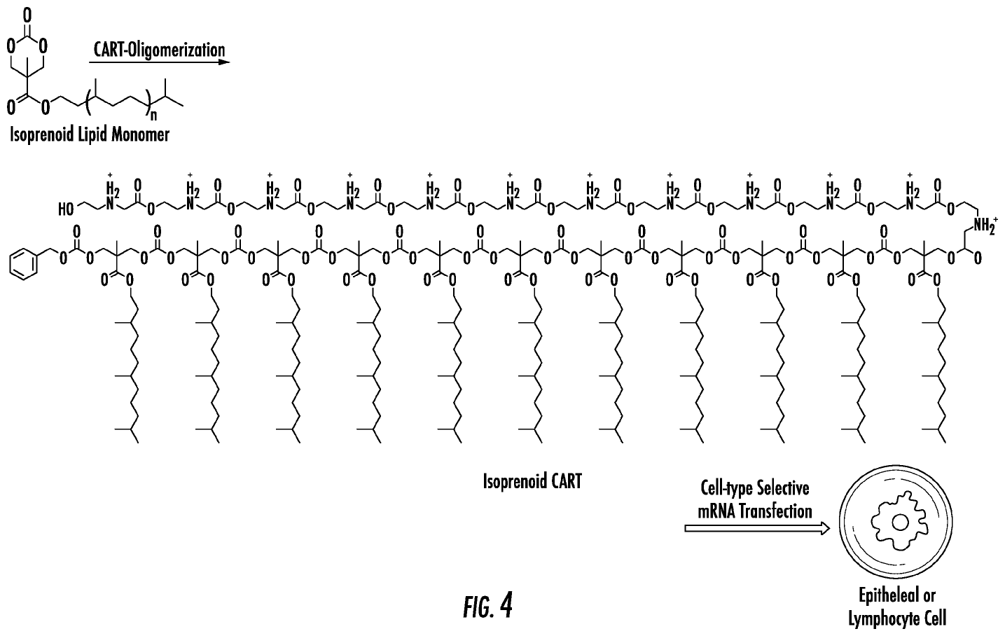

The branched lipid CARTs described herein are differentiated from other CARTs previously described by their non-linear lipidation. Isoprenoids employ regular methylation to force a mild curvature and decreased melting point. Stereo-enrichment of isoprenoid lipids is possible through selective hydrogenation. Forked lipid CARTs have longer hydrophobic branches yielding a conical geometry. Branches can be symmetrical or asymmetrical, contain isoprenoid-like alkylation patterns, or alkene-type unsaturation. Cyclic lipids are readily accessible and represent a similar form of branching. As discussed in more detail below, using branched lipid CARTs we have also explored compounds having up to 50 total repeat units, and 2:1 lipid repeat unit to cationic repeat unit ratios. Exemplary isoprenoid based lipids in charge-altering releasable transporters (CARTS) is shown in FIG. 4.

Hydrogenated isoprenoid lipids, lacking unsaturated double bonds, are more stable than oleyl and nonenyl lipids. This removes the need to acquire >99% pure oleyl alcohol. In addition, and in contrast to ONA (ONA290), which performs well in many cell-types, isoprenoids demonstrate efficacy in specific cell types. Further, isoprenoids are cheap starting materials, bio-inspired (archaea), and atypical in commercial transfection reagents.

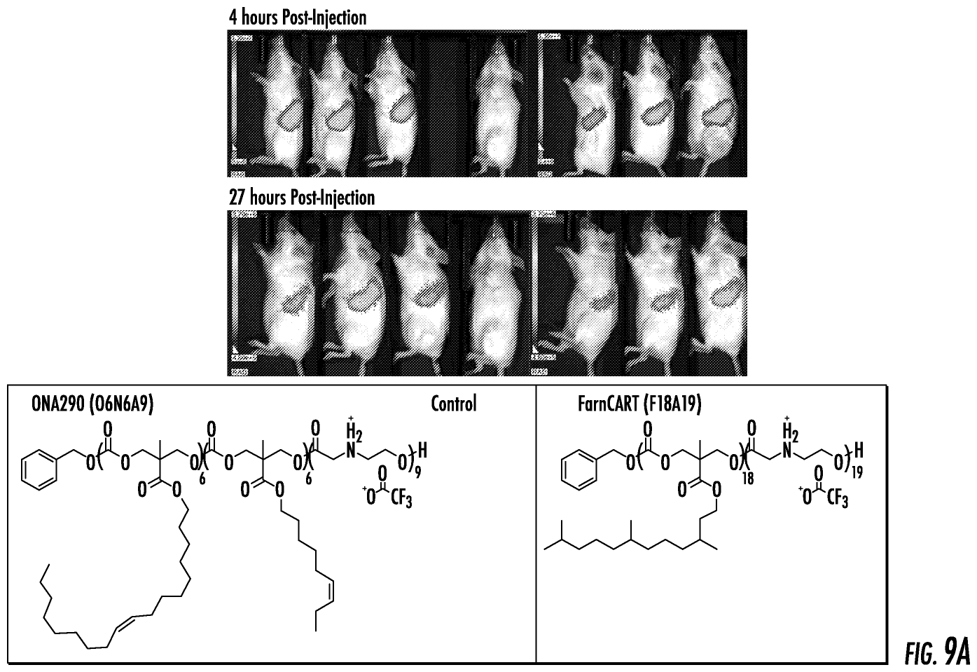

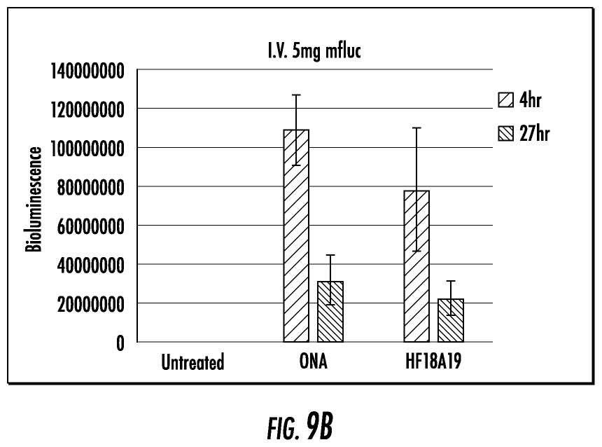

As discussed in more detail below, unmixed isoprenoid CARTs outcompete a mixed lipid CART (ONA290, structure shown in FIG. 9A) in tested cell-lines and allow for selective transfection of immortalized adherent cell lines and selective transfection of immortalized lymphocytes in vitro based on the lipid identity. Further, isoprenoid CARTs can efficaciously transfect murine models in vivo.

### Methods

BLI of Fluc mRNA Delivery to Various Cell Lines

HeLa cells and A549 were seeded at 15,000 cells per well in 100 μL of serum-containing DMEM media (10% fetal bovine serum, 1% penicillin/streptomycin) in white 96-well plates. Jurkat and GM12878 cells were seeded at 50,000 cells per well in 100 μL D-luciferin solution (300 μg/mL) in serum-free RPMI media in white 96-well plates. Adherent cells were allowed to adhere overnight while lymphocyte lines were transfected immediately. Adherent cells were washed with serum-free DMEM media next day before treatment, after which 50 μL of D-luciferin solution (300 μg/mL) in serum-free DMEM was added to each well.

Oligomer/mRNA polyplexes were prepared by mixing RNase-free PBS pH 5.5 and luciferase mRNA with various amounts of oligomer from 2 mM DMSO stock solutions, to achieve specific cooligomer/mRNA ratios (optimized to a theoretical cation:anion ratio of 10:1). The complexes were prepared by rapidly mixing with micropipette for 20 s at room temperature before treatment. The Lipo control was prepared in OptiMEM per the manufacturer's instructions. 1 μL of the mRNA/cooligomer complexes was added to a total volume of 50 or 100 μL/well for a final mRNA concentration of 30 ng/well. All conditions were performed in replicates of four. Cells were incubated with treatment for 5-7 h at 37° C.

EGFP mRNA Delivery and Expression in HeLa Cells by Flow Cytometry

HeLa cells and A549 were seeded at 40,000 cells per well in 100 μL of serum-containing DMEM media in white 96-well plates. Jurkat and GM12878 cells were seeded at 100,000 cells per well in serum-free RPMI media in white 96-well plates. Adherent cells were allowed to adhere overnight while lymphocyte lines were transfected immediately. Adherent cells were washed with serum-free DMEM media the next day before treatment, after which 50 μL of serum-free DMEM was added to each well.

Oligomer/mRNA polyplexes were prepared by mixing RNase-free PBS pH 5.5 and eGFP mRNA with various amounts of oligomer from 2 mM DMSO stock solutions, to achieve specific cooligomer/mRNA ratios (optimized to a theoretical cation:anion ratio of 10:1). The complexes were prepared by rapidly mixing with micropipette for 20 s at room temperature before treatment. The Lipo control was prepared in OptiMEM per the manufacturer's instructions. 1.65 μL of the mRNA/cooligomer complexes was added to a total volume of 50 or 100 μL/well for a final mRNA concentration of 100 ng/well. All conditions were performed in replicates of four. Cells were incubated with treatment for 5-7 h at 37° C.

The adherent cells were incubated for 8 h at 37° C. then trypsinized with 50 μL of trypsin-EDTA (0.05%) for 10 min at 37° C., after which 50 μL of serum-containing DMEM was added to each well to quench the trypsin. The nonadherent cells were analyzed with flow cytometry immediately. The treated wells were read on a flow cytometry analyzer (Quenteon at Stanford University). The data presented are the geometric mean fluorescent signals from 5-10 k cells analyzed. For transfection efficiency, untreated cells were gated for no EGFP expression, and the data presented are the percentages of cells analyzed with higher EGFP expression than untreated cells.

### Results

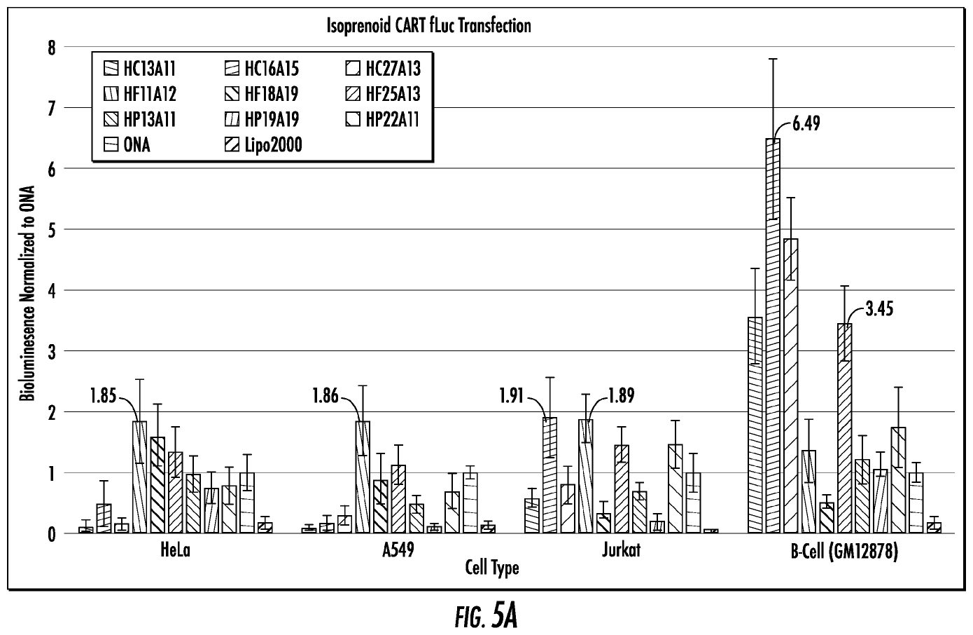

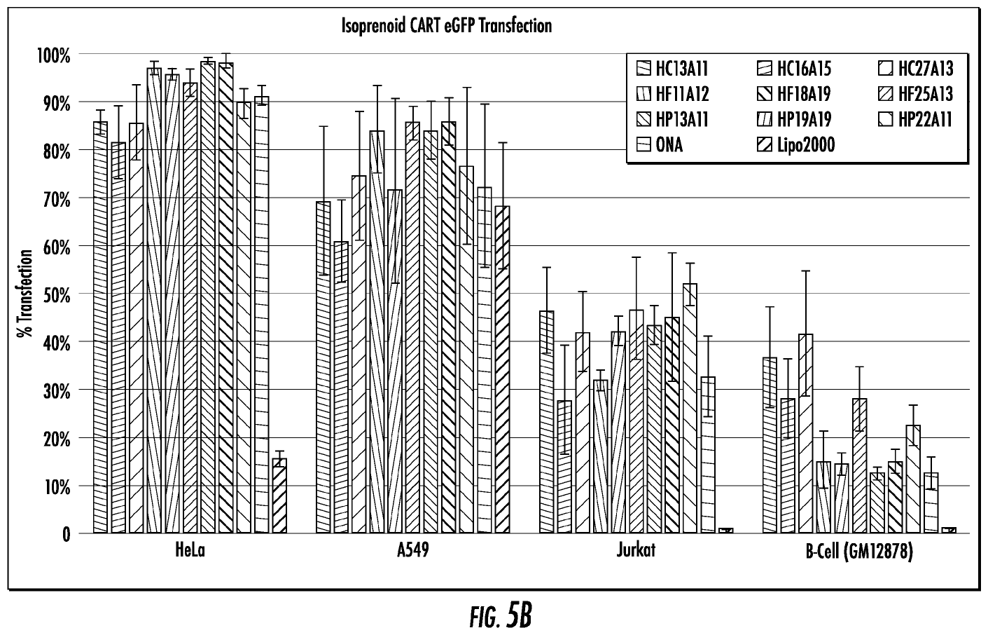

Several observations have been made in FIGS. 5A-5B. HF11A12 is consistently a top performer at about 1.9-fold ONA290 in three of four cell lines. In each cell line there are multiple isoprenoid CARTs which outperform ONA290 and several which are comparable. In adherent cell lines, FamCARTs tend to be the best performers followed by PhytCARTs. CitroCARTs do not perform well in adherent cell-lines but perform exceptionally well in B-Cells compared to ONA290. 2:1 Lipid to amine CARTs show great activity in non-adherents even when the corresponding 1:1 CART with the same lipid does not perform well. When deciding which CARTs are the best, there are two criteria to examine 1) the cell type activity, and 2) the cell-class selectivity. The best CARTs for HeLa and A549 are F11A12, F18A19, and F25A13. The best CARTs for Jurkat are F11A12, C16A15, F25A13 and P22A11. The best CARTs for GM12878 and C13A11, C16A15, C27A13, and F25A13. If one desired to select a transporter for its selectivity one would elect to utilize F18A19 for transfecting adherent cells in the presence of non-adherents. The best transporters for non-adherent selectivity is C16A15 or C27A13

In HeLa, A549, and Jurkat Cells there is relatively consistent transfection regardless of transporter identity (HeLa: ˜92%; A549: ˜76%; Jurkat: ˜41%). Lipofectamine2000 is ineffective in non-adherent cell-lines and only show minimal transfection in A549 Cells. Only in B-Cells there is a close correlation with percent Transfection and fLuc Bioluminescence.

In Vivo fLuc mRNA Transfection

We performed and in vivo transfection using murine models injected via the tail-vein with fLuc mRNA then at the time of imagine firefly luciferin is injected intraperitoneally. This experiment was conducted with three isoprenoid transporters showing efficacy in vitro. We selected C17A16, F18A19, and P10A9 for the first experiments in early 2021 to ensure efficacy would be observed. Indeed, it made us very hopeful of this transporter class. We saw spleen selectivity from all transporters, as is common in CARTs. In farnesol and phytanol transporters, overflow to the liver was also observed. We also observed the citronellol functionalized transporter was less efficacious in vivo than in vitro, suggesting the transporter isn't as stable as the others, or it is transfecting less epithelial cells than the farnesol and phytanol functionalized transporters. All subjects behaved normally following injection and no signs of toxicity were observed.

A follow up experiment compared F18A19 to ONA290. We found that the total bioluminescence was approximately 75% that of ONA290's at the 4-hour time point and again at the 27 hour time point (FIG. 9A). The final intravenous injection results are also shown in FIG. 9B.

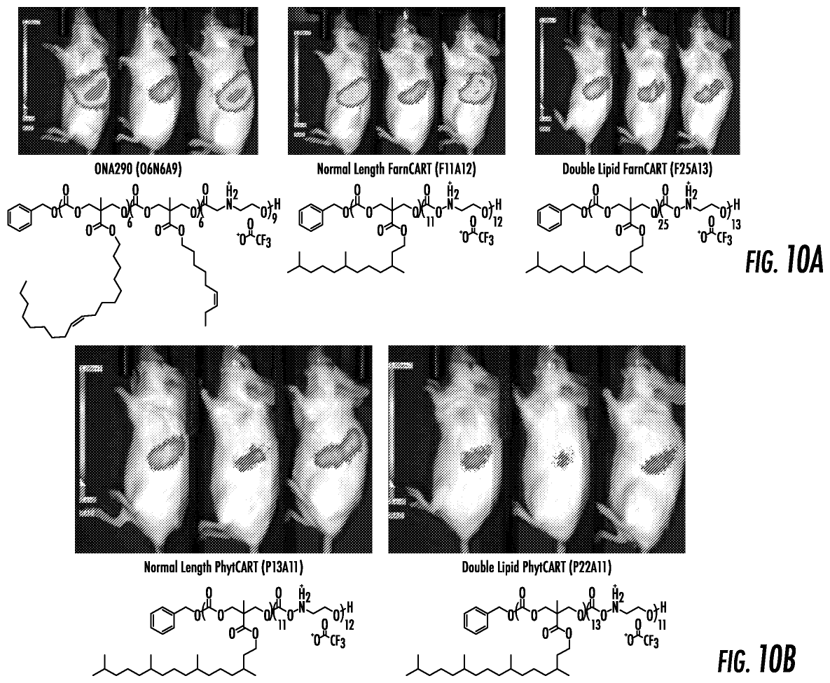

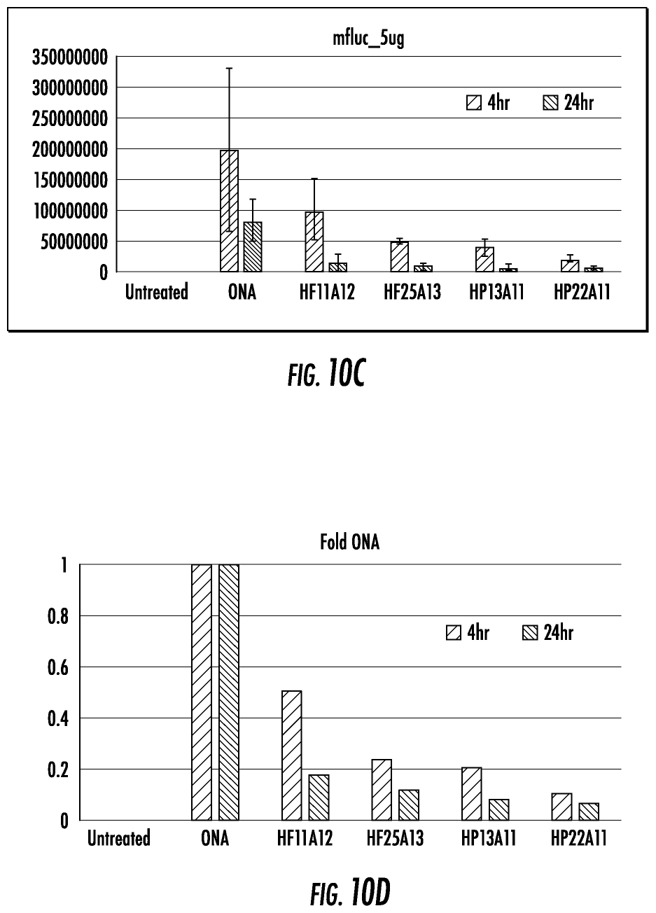

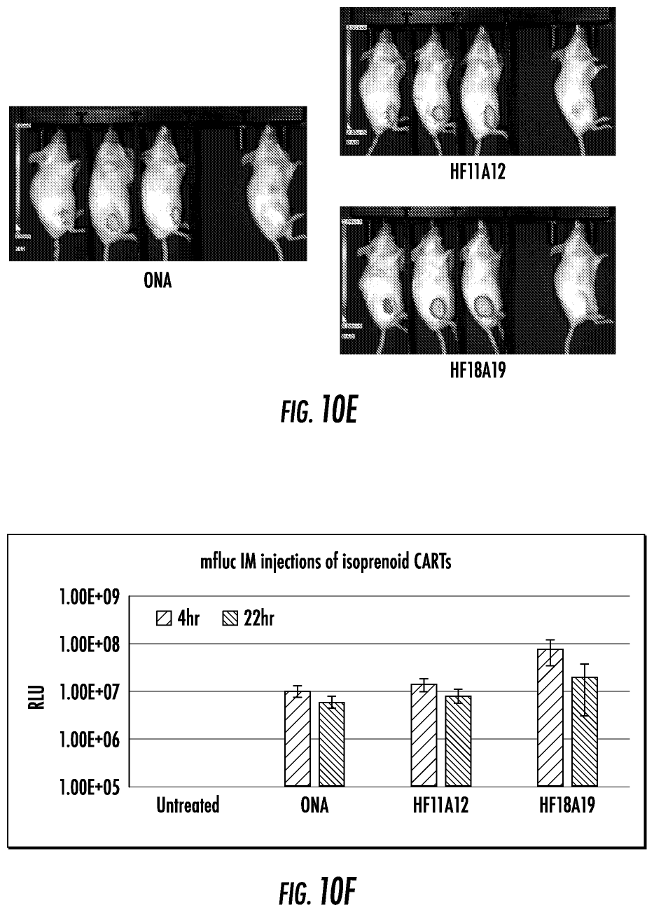

Tests results for additional CART F11A12, F25A13, P13A11, and P22A11 are also shows in FIGS. 10A-10F. The normal length oligomers generally show very good activity in vitro and the double lipid oligomers have great activity as well as interesting particle characteristics. All mice looked healthy and robust signal from the spleen is observed from both farnesol CART (FIGS. 10A-10B). After 24 hrs, lower expression was observed than at the 4 hr time point (FIGS. 10C-10D).

We performed i.m. injections of HF11A12 and HF18A19 and evaluate their performance with luciferase mRNA delivery, in comparison with ONA CART (ONA290). Representative images after injections are shown below. HF11A12 performs similarly as ONA290, while HF18A19 achieves 8-fold activity 4 hr after injection compared to ONA290 (FIGS. 10E-10F).

### Example 6: Glycerol Based Lipids in Charge-Altering Releasable Transporters (CARTs)

Branched glycerol CARTs are differentiated by their non-linear lipidation and the incorporation of glycerol, mimicking physiological lipid structures. Branched glycerol CARTs have long hydrophobic branches, yielding a conical geometry. Branches can be symmetrical or asymmetrical, contain isoprenoid-like alkylation patterns, or alkene-type unsaturation. Cyclic lipids are readily accessible and are expected to provide a similar form of branching.

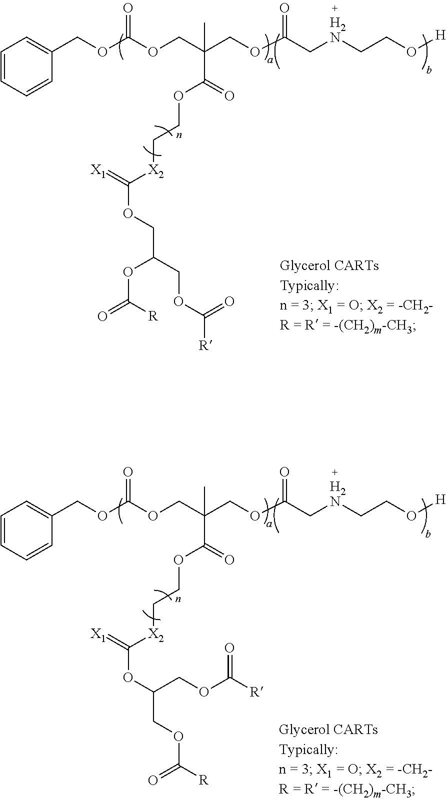

We examined the effects on glycerol lipid CARTs of incorporating up to 50 total repeat units.

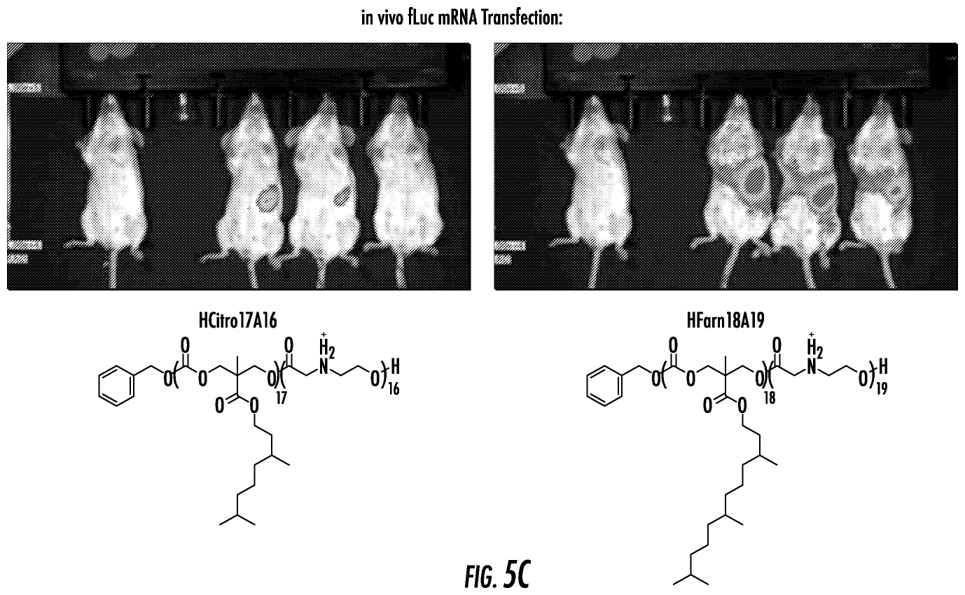

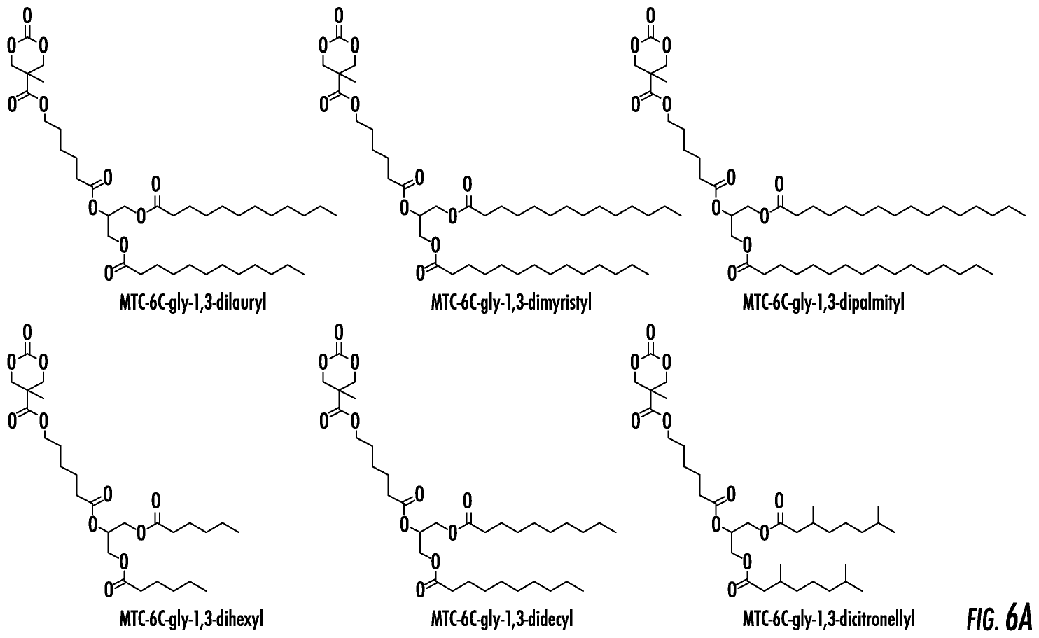

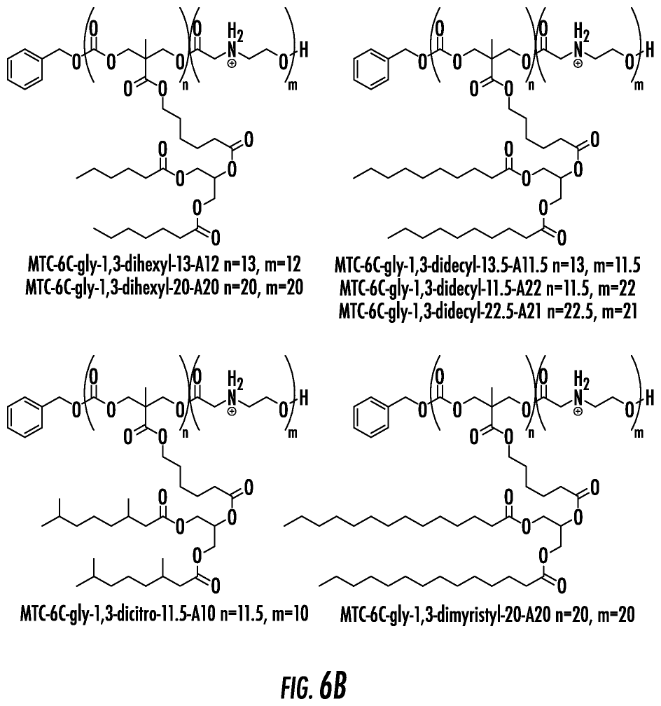

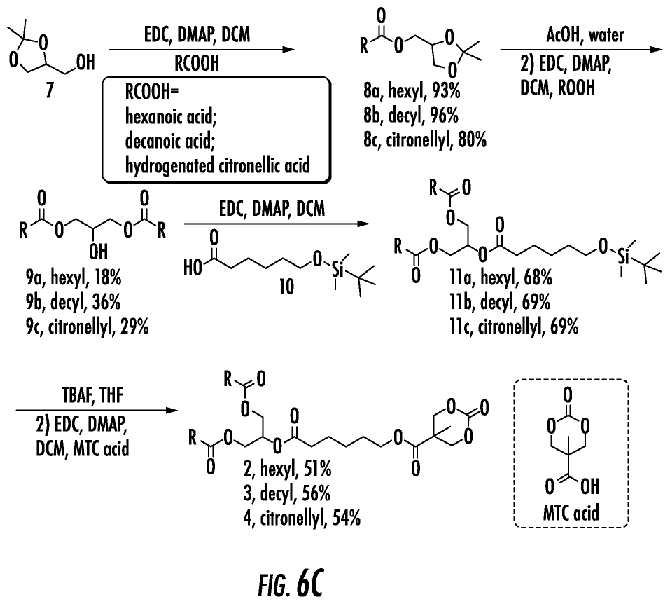

Using a previously developed synthetic route, new MTC-glycerol monomers were synthesized to probe the effect of incorporating longer lipids into MTC-glycerol monomers. Exemplary monomers are shown in FIG. 6A and exemplary CARTs are shown in FIG. 6B.

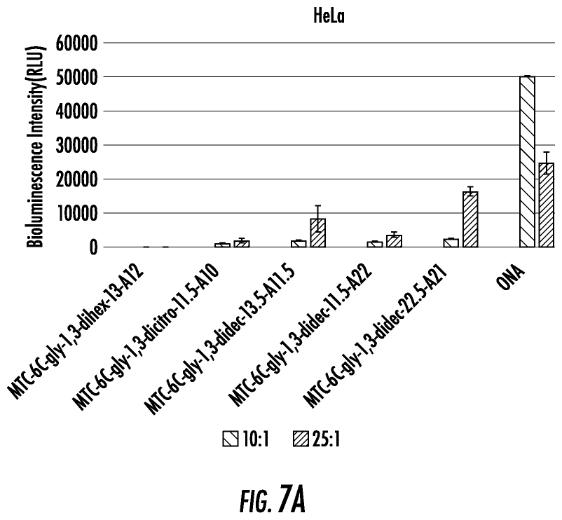

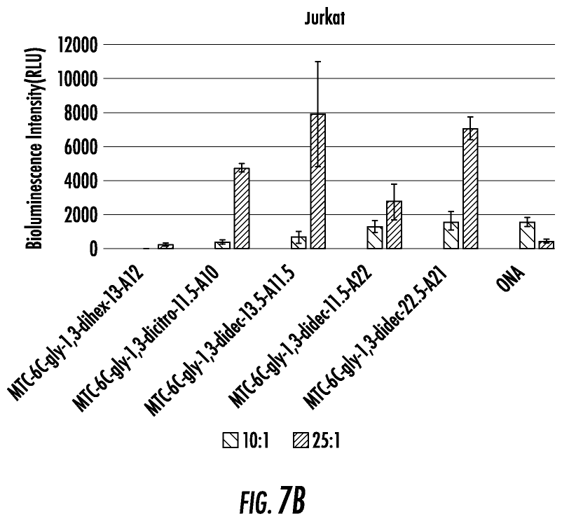

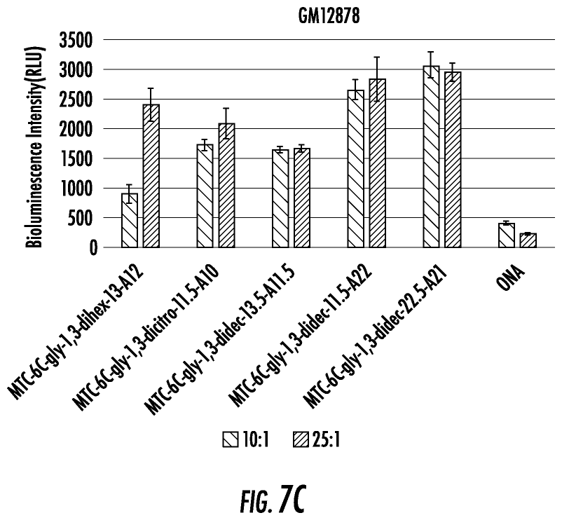

As shown in FIG. 7A, MTC-glycerol CARTs (10:1) were showing about 2-5% ONA290 (10:1) activity while at 25:1+/−charge ratio, showing activities that range from 4% to 32% as ONA290 (10:1) in HeLa. In FIG. 7B, MTC-glycerol diCitro and diDecyl CARTs(25:1) demonstrated 1.72-4.93 fold activity as ONA290 (10:1) in Jurkats with MTC-6C-gly-1,3-didec-13.5-A11.5 and MTC-6C-gly-1,3-didec-22.5-A21 being the best performers. In FIG. 7C, all MTC-glycerol CARTs tested at both charge ratios outperformed ONA290 (10:1) in GM12878, by 2.16 to 7.44 fold, with MTC-6C-gly-1,3-didec-11.5-A22 and MTC-6C-gly-1,3-didec-22.5-A21 being the best performers.

In vitro evaluation of MTC-glycerol CARTs were conducted in conditions of 30 ng fluc mRNA/well and 50 k cell per well, and CARTs were screened at 10:1 and 25:1 charge ratio.

Fold-ONA analysis of MTC-glycerol CARTs in three cell line tested are shown in Table below.

TABLE 1
Fold ONA in HeLa

| CARTs tested | 10:1 | 25:1 |
|---|---|---|
| MTC-6C-gly-1,3-dihex-13-A12 | 0.00 | 0.00 |
| MTC-6C-gly-1,3-dicitro-11.5-A10 | 0.02 | 0.04 |
| MTC-6C-gly-1,3-didec-13.5-A11.5 | 0.04 | 0.16 |
| MTC-6C-gly-1,3-didec-11.5-A22 | 0.03 | 0.08 |
| MTC-6C-gly-1,3-didec-22.5-A21 | 0.05 | 0.32 |
| ONA290 | 1.00 | 0.49 |

TABLE 2
Fold ONA in Jurkat

| CARTs tested | 10:1 | 25:1 |
|---|---|---|
| MTC-6C-gly-1,3-dihex-13-A12 | 0.04 | 0.17 |
| MTC-6C-gly-1,3-dicitro-11.5-A10 | 0.27 | 2.95 |
| MTC-6C-gly-1,3-didec-13.5-A11.5 | 0.42 | 4.93 |
| MTC-6C-gly-1,3-didec-11.5-A22 | 0.82 | 1.72 |
| MTC-6C-gly-1,3-didec-22.5-A21 | 1.04 | 4.41 |
| ONA290 | 1.00 | 0.28 |

TABLE 3
Fold ONA in GM12878:

| CARTs tested | 10:1 | 25:1 |
|---|---|---|
| MTC-6C-gly-1,3-dihex-13-A12 | 2.16 | 5.83 |
| MTC-6C-gly-1,3-dicitro-11.5-A10 | 4.15 | 5.04 |
| MTC-6C-gly-1,3-didec-13.5-A11.5 | 3.97 | 4.04 |
| MTC-6C-gly-1,3-didec-11.5-A22 | 6.43 | 6.89 |
| MTC-6C-gly-1,3-didec-22.5-A21 | 7.44 | 7.17 |
| ONA290 | 1.00 | 0.54 |

DLS and Zeta information on selected MTC-glycerol CARTs at different charge ratio are shown in Tables below.

TABLE 4
DLS

| | Charge | Average | |
|---|---|---|---|
| CART Name | ratio | diameter(nm) | PDI |
| MTC-6C-gly-1,3-didecyl-13.5- | 10:1 | 287.5 | 0.181 |
| MTC-6C-gly-1,3-didecyl-13.5- | 25:1 | 222.3 | 0.244 |
| MTC-6C-gly-1,3-didecyl-22.5- | 10:1 | 139.9 | 0.173 |
| MTC-6C-gly-1,3-didecyl-22.5- | 25:1 | 103.0 | 0.215 |

TABLE 5
Zeta

| CART Name | T0 | T1 | | T2 | |
|---|---|---|---|---|---|
| MTC-6C-gly-1,3-didecyl-22.5- | 0 min | 10 | min | 18 | min |
| A21 | 92.61 | 25.11 | mV | 30.07 | |
| MTC-6C-gly-1,3-didecyl-13.5- | 0 min | 5 | min | 10 | min |

| | | | | |
|---|---|---|---|---|
| A11.5 | 32.44 | 28.76 | 30.07 | mV |

Transfection efficiency information on selected MTC-glycerol CARTs was measured in conditions of 100 ng eGFP mRNA/well, 100 k Jurkat cells in 100 ul of media per well, and CARTs screened at 10:1/25:1 charge ratio.

TABLE 6
Transfection efficiency

| CARTs tested | 10:1 | 25:1 |
|---|---|---|
| ONA290 | 39.4% | NA |
| MTC-6C-gly-1,3-didec-13.5- | 63.3% | 83.1% |
| MTC-6C-gly-1,3-didec-22.5-A21 | 79.2% | 74.6% |

### Example 7: Characterization of Nanoparticles

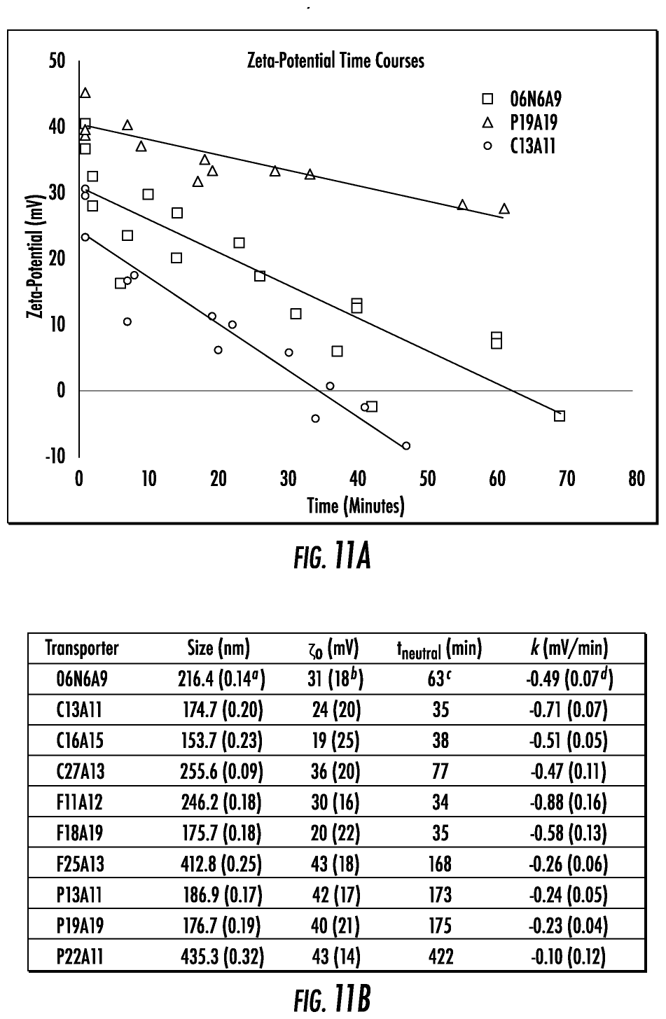

The co-oligomers, O6N6A9, P19A19, and C13A11, are complexed with nucleic acid and condense to form nanoparticles. These nanoparticles are characterized as shown FIG. 11A-11B.

FIG. 11A shows ζ(zeta)-potential time course for three representative transporters, O6N6A9, P19A19, and C13A11 shown (n=3). Each time course is globally fitted with a linear regression to determine k, tneutral and ζ₀. Data for each transporter in this study is shown in FIG. 11B. The value contained within the parentheses is the nanoparticle's average polydispersity index. The value contained within the parentheses represents the average standard deviation of the nanoparticle formulation's initial ζ-potentials. tₙₑᵤₜᵣₐₗ is defined as the x-intercept of the global linear regression of the ζ-potential's time course for each transporter. The value contained within the parentheses is the standard error associated with the variable k as derived from the global linear regression.

### REFERENCES

1. Colin J. McKinlay, Jessica R. Vargas, Timothy R. Blake, Jonathan W. Hardy, Masamitsu Kanada, Christopher H. Contag, Paul A. Wender*, and Robert M. Waymouth* “Charge-altering releasable transporters (CARTs) for the delivery and release of messenger RNA in living animals” Proc. Natl. Acad. Sci. USA 2017 E448-456, doi/10.1073/pnas.1614193114
2. Benner, Nancy; Near, Katherine; Bachmann, Michael; Contag, Christopher; Waymouth, Robert; Wender, Paul “Functional DNA Delivery Enabled by Lipid-Modified Charge-Altering Releasable Transporters (CARTs)” Biomacromolecules 2018 19 (7), pp 2812-2824 DOI: 10.1021/acs.biomac.8b00401
3. Colin J. McKinlay, Nancy L. Benner, Ole A. Haabeth, Robert M. Waymouth, and Paul A. Wender “Enhanced mRNA delivery into lymphocytes enabled by lipid-varied libraries of charge-altering releasable transporters” Proc. Natl. Acad. Sci. USA 2018, 115 (26), E5859-E5866.
4. Ole A. W. Haabeth, Timothy R. Blake, Colin J. McKinlay, Robert M. Waymouth, Paul A. Wender, Ronald Levy “mRNA Vaccination with Charge-Altering Releasable Transporters elicit human T cell responses, and Cures Established Tumors in mice” Proc. Natl. Acad. Sci. USA 2018 115 (39), E9153-E9161.
5. Ole A. W. Haabeth, Timothy R. Blake, Colin J. McKinlay, Anders Tveita, Robert M. Waymouth, Paul A. Wender, Ronald Levy “Local OX40L, CD80, and CD86 mRNA delivery kindles global anti-cancer immunity” Cancer Research 2019, vol 79 (7), 1624-1634. doi: 10.1158/0008-5472.CAN-18-2867; PubMed 30692215
6. Nancy L. Benner, Rebecca L. McClellan, Christopher R. Turlington, Ole A Haabeth Robert M. Waymouth, Paul A. Wender “Synthesis of Poly(serine esters) by Organocatalytic Ring Opening Polymerization and their use as Charge-Altering Releasable Transporters for mRNA Delivery in vitro and in vivo” Journal of the American Chemical Society 2019, 141, 21, 8416-8421 DOI: 10.1021/jacs.9b03154
7. Habibian, Maryam; McKinlay, Colin; Blake, Timothy; Kietrys, Anna; Waymouth, Robert; Wender, Paul; Kool, Eric* “Reversible RNA Acylation for Control of CRISPR-Cas9 Gene Editing” RSC Chemical Science 2019, 11, 1011-1016. DOI: 10.1039/C9SC03639C.
8. Blake, Timothy; Ho, Wilson; Christopher R. Turlington, Xiaoyu Zang, Melanie Huttner, Wender, Paul, Waymouth, Robert “Synthesis and Mechanistic Investigations of pH-Responsive Cationic Poly(aminoester)s” Chemical Science 2020 11, 2951-2966 DOI: 10.1039/c9sc05267d
9. Aaron J. Wilk, Nancy L. Weidenbacher, Rosemary Vergara, Ole A. W. Haabeth, Ronald Levy, Robert M. Waymouth, Paul A. Wender, and Catherine A. Blish “Charge-Altering Releasable Transporters enable Phenotypic Manipulation of Natural Killer Cells for Cancer Immunotherapy” Blood Advances 2020, 4 (17): 4244-4255.
10. Haabeth, Ole; Lohmeyer, Julian; Sallets, Adrienne; Blake, Timothy; Sagiv-Barfi, Idit; Czerwinski, Debra; McCarthy, Blaine; Powell, Abigail; Wender, Paul; Waymouth, Robert; Levy, Ronald “An mRNA SARS-CoV-2 vaccine employing Charge-Altering Releasable Transporters with a TLR-9 agonist induces neutralizing antibodies and T cell memory” ACS Central Science 2021, 7, 1191-1204. DOI: 10.1021/acscentsci.1c00361

1. A co-oligomer comprising non-linear branched lipophilic monomers (LP) and poly(alpha-aminoester) monomers (AM), wherein the co-oligomer has a formula:
R^{1A}-[L¹-[(LP¹)_{z1}-(LP²)_{z3}-(AM)_{z2}]_{z4}-L²-R^{2A}]_{z5} (I), or
R^{1A}-[L¹-[(LP¹)_{z1}-(AM)_{z2}-(LP²)_{z3}]_{z4}-L²-R^{2A}]_{z5} (I′)
wherein:
R^{1A} and R^{2A} are each independently hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are optional linkers and are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each LP¹ or LP² is independently a non-linear branched lipophilic monomer having a structure of: 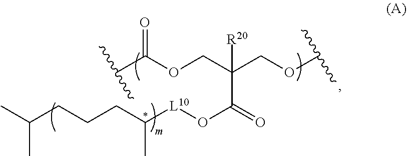
wherein:
m is an integer from 0 to 6;
L¹⁰ is independently a substituted or unsubstituted alkylene; and
R²⁰ is independently hydrogen or unsubstituted C₁-C₄ alkyl,
LP¹ and LP² are same or different;
AM is a poly(alpha-aminoester) monomer;
z5 is an integer from 1 to 10;
z1 and z3 are independently integers from 5 to 30, wherein at least one of z1, or z3 is not 0;
z4 is an integer from 1 to 100, and
z2 is an integer from 5 to 30.

2. The co-oligomer of claim 1, wherein L¹⁰ is independently an unsubstituted C₁-C₄ alkylene.

3. (canceled)

4. The co-oligomer of claim 1, wherein each LP¹ and LP² is independently

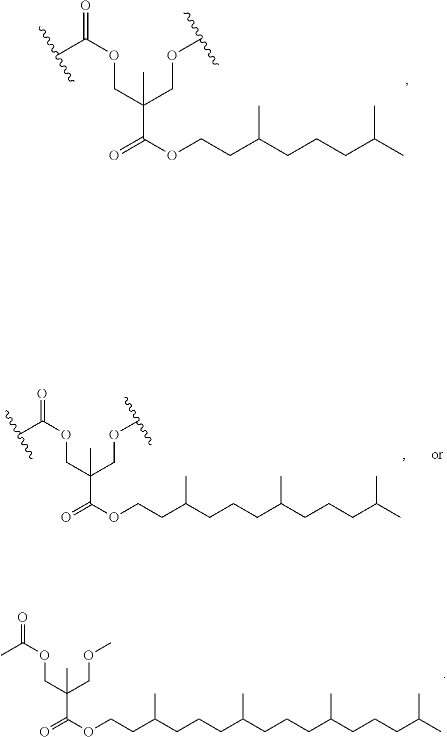

5. A co-oligomer comprising non-linear branched lipophilic monomers (LP) and cationic poly(alpha-aminoester) monomers (AM), wherein the co-oligomer has a formula:
R^{1A}-[L¹-[(LP¹)_{z1}-(LP²)_{z3}-(AM)_{z2}]_{z4}-L²-R^{2A}]_{z5} (I), or
R^{1A}-[L¹-[(LP¹)_{z1}-(AM)_{z2}-(LP²)_{z3}]_{z4}-L²-R^{2A}]_{z5} (I′)
wherein
R^{1A} and R^{2A} are each independently hydrogen —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each LP¹ or LP² are independently a non-linear branched lipophilic monomer having a structure of: 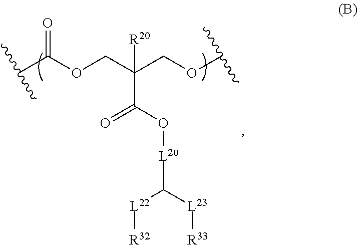
wherein:
L²⁰ is -L^{20A}-L^{20B}-;
each L^{20A} and L^{20B} is independently a bond, a substituted or unsubstituted alkylene, substituted or unsubstituted heteroalkylene, substituted or unsubstituted cycloalkylene, or substituted or unsubstituted heterocycloalkylene;
each L²², and L²³ is independently a bond, substituted or unsubstituted alkylene, or substituted or unsubstituted heteroalkylene;
R²⁰ is independently hydrogen or unsubstituted C₁-C₄ alkyl; and
each R³² and R³³ are independently hydrogen, or substituted or unsubstituted alkyl,
LP¹ and LP² are same or different;
AM is said poly(alpha-aminoester) monomer;
z1 and z3 are independently integers from 5 to 30, wherein at least one of z1, or z3 is not 0;
z4 is an integer from 1 to 100, and
z2 is an integer from 5 to 30.

6. The co-oligomer of claim 5, wherein:
L²² is independently a bond, unsubstituted C₁-C₄ alkylene, -L²⁴-OC(O)—, or -L²⁴-C(O)O—;
L²³ is independently a bond, unsubstituted C₁-C₄ alkylene, -L²⁵-OC(O)—, or -L²⁵-C(O)O—; and
each L²⁴ and L²⁵ are independently a bond or substituted or unsubstituted C₁-C₃ alkylene.

7. The co-oligomer of claim 5, wherein L²⁰ is independently a bond, unsubstituted C₁-C₁₀ alkylene, -L^{21A}-O—, —O-L^{21B}-, -L^{21A}-O-L^{21B}-, -L^{21A}-C(O)—O—, —C(O)O-L^{21B}-, -L^{21A}-C(O)O-L^{21B}-, -L^{21A}-OC(O)—, —OC(O)-L^{21B}-, -L^{21A}-OC(O)-L^{21B}-, -L^{21A}-O—C(O)—O—, —O—C(O)—O-L^{21B}-, or -L^{21A}-O—C(O)—O-L^{21B}-, and
each L^{21A} and L^{21B} is independently a bond or substituted or unsubstituted C₁-C₁₀ alkylene.

8. The co-oligomer of claim 5, wherein each LP¹ and LP² independently has a structure of: 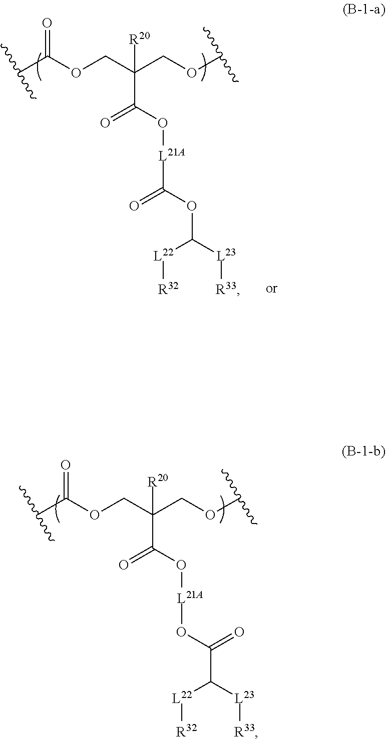
wherein L^{21A} is independently unsubstituted C₅-C₁₀ alkylene.

9. The co-oligomer of claim 5, wherein each LP¹ and LP² independently has a structure of: 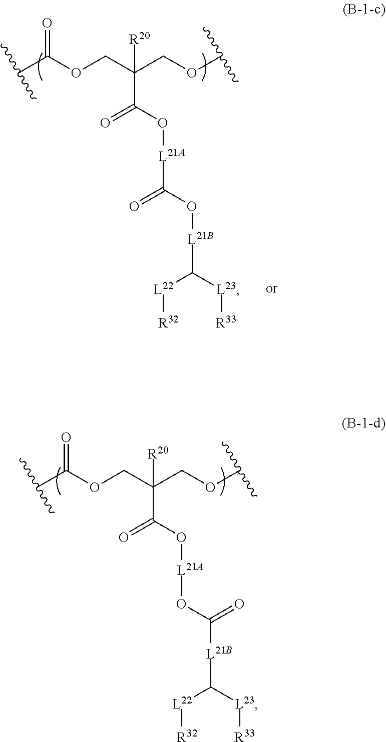
wherein L^{21A} is independently unsubstituted C₅-C₁₀ alkylene; and L^{21B} is independently a bond or unsubstituted C₁-C₃ alkylene.

10. The co-oligomer of claim 5, wherein each LP¹ and LP² independently has a structure of: 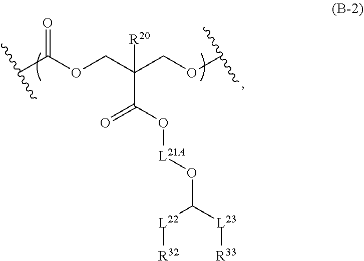
wherein L^{21A} is independently unsubstituted C₅-C₁₀ alkylene.

11. The co-oligomer of claim 5, wherein each LP¹ and LP² independently has a structure of 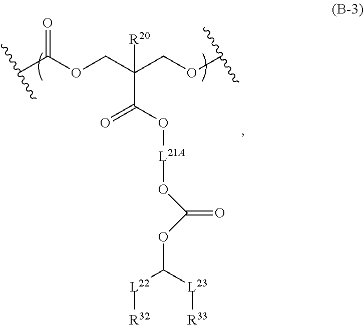
wherein L^{21A} is independently unsubstituted C₅-C₁₀ alkylene.

12. The co-oligomer of claim 8, wherein:
L²² is independently a bond or unsubstituted C₁-C₄ alkylene; and
L²³ is independently a bond or unsubstituted C₁-C₄ alkylene.

13. The co-oligomer of claim 5, wherein each LP¹ and LP² independently has a structure of 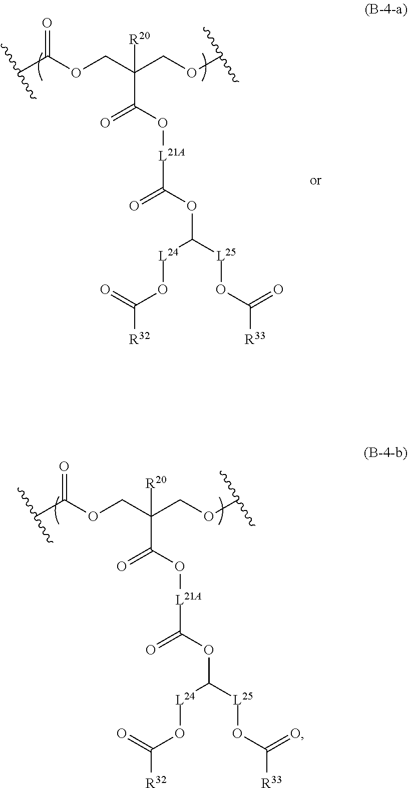
wherein L^{21A} is independently unsubstituted C₅-C₁₀ alkylene.

14. The co-oligomer of claim 5, wherein each LP¹ and LP² independently has a structure of:

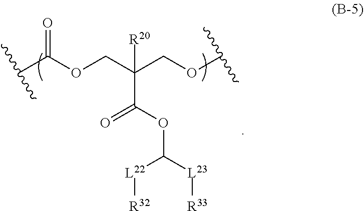

15. The co-oligomer of claim 5, wherein each LP¹ and LP² independently has a structure of: 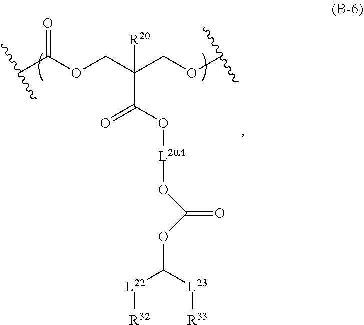
wherein L^{20A} is substituted or unsubstituted cycloalkylene, or substituted or unsubstituted heterocycloalkylene.

16. The co-oligomer of claim 5, wherein R²⁰ is methyl.

17. The co-oligomer of claim 5, wherein each R³² and R³³ are unsubstituted saturated or unsaturated C₄-C₂₀ alkyl.

18. (canceled)

19. (canceled)

20. The co-oligomer of claim 5, wherein each LP¹ and LP² is independently

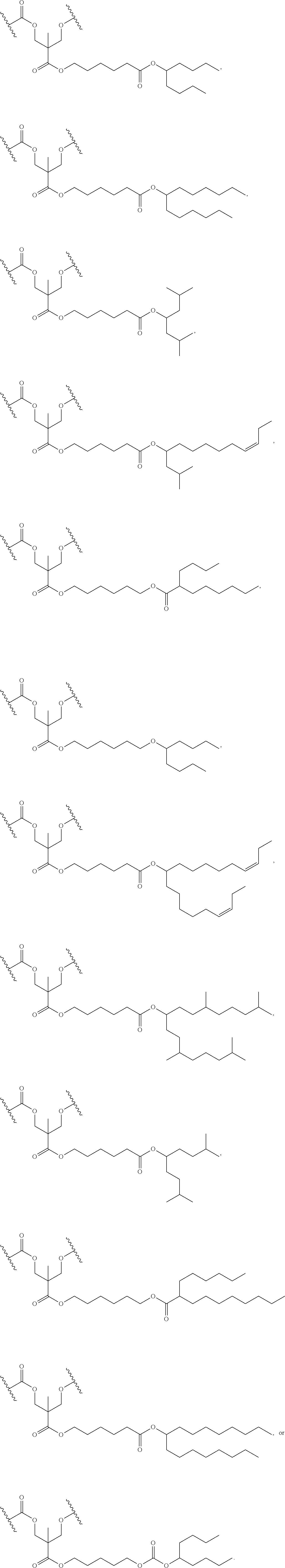

21. The co-oligomer of claim 5 wherein each LP¹ and LP² is independently

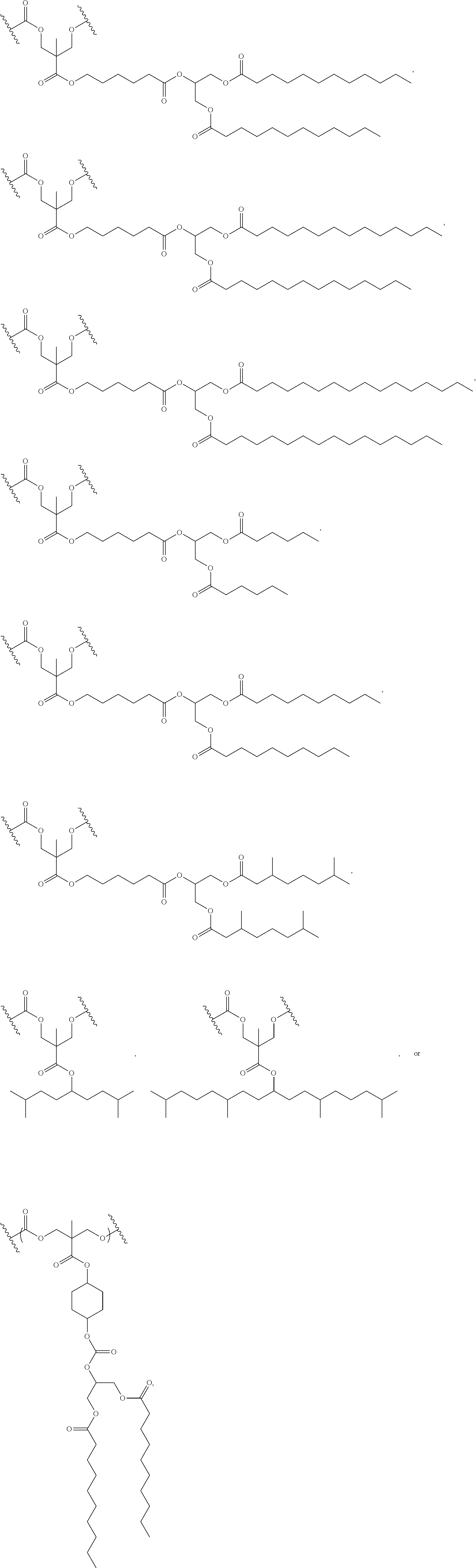

22-33. (canceled)

34. The co-oligomer of claim 1, wherein said poly(alpha-aminoester) monomer has the formula: 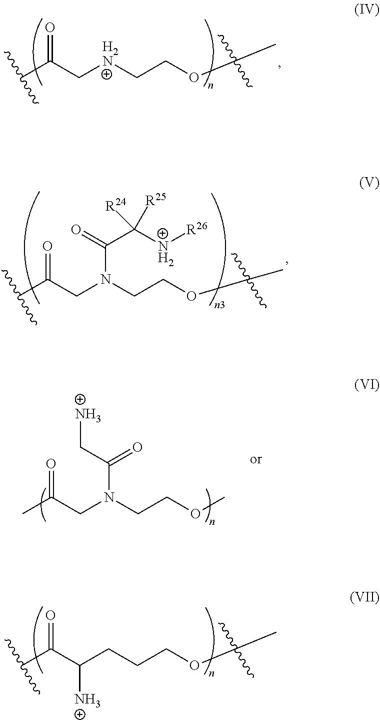
wherein
in formula V R²⁴, R²⁵ and R²⁶ are independently hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and n3 is an integer from 2 to 50; and
in formula IV, VI, and VII n is an integer of 2 to 50.

35-37. (canceled)

38. The co-oligomer of claim 1, wherein the co-oligomer is 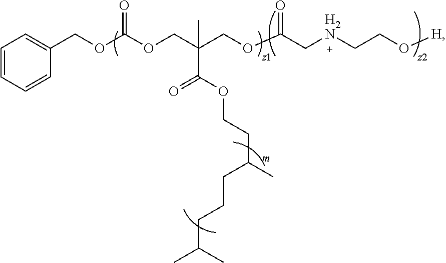
wherein m is 12, or 3;
each z1 and z2 is independently an integer from 5 to 30.

39. A composition comprising a co-oligomer of claim 1 complexed with nucleic acid wherein the nucleic acid is non-covalently bound to the co-oligomer.

40. The composition of claim 39, wherein said nucleic acid is a messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA).

41. A nanoparticle composition comprising the composition of claim 39.

42. A pharmaceutical composition comprising the composition of claim 39, and a pharmaceutically acceptable carrier.

43. A method of transfecting a nucleic acid into a cell ex vivo, the method comprising contacting a cell with the composition of claim 39.

44. A method of delivering a nucleic acid to a cell in a subject, the method comprising administering the pharmaceutical composition of claim 42 to the subject.

45. The method of claim 44, wherein said nucleic acid is RNA or DNA.

46. The method of claim 45, wherein the nucleic acid is an RNA selected from a messenger RNA (mRNA), a small interference RNA (siRNA), a short hairpin RNA (shRNA), a micro RNA (miRNA), a guide RNA (gRNA), a CRISPR RNA (crRNA), and a transactivating RNA (tracrRNA).

47. The method of claim 45, wherein the nucleic acid is a DNA selected from a plasmid DNA (pDNA), a minicircle DNA, and a genomic DNA (gNDA).

48-73. (canceled)
